(19) United States
(12) Patent Application Publication
(10) Pub. No.: US 2023/0093476 A1
(43) Pub. Date: Mar. 23, 2023

(54) SUBSTITUTED FUSED IMIDAZOLE DERIVATIVES AND METHODS OF TREATING REFRACTIVE OCULAR DISORDERS

(71) Applicant: vTv Therapeutics LLC, High Point, NC (US)

(72) Inventor: Otis Clinton ATTUCKS, Winston-Salem, NC (US)

(21) Appl. No.: 17/759,977

(22) PCT Filed: Feb. 1, 2021
(86) PCT No.: PCT/US21/16018

§ 371 Date: Aug. 2, 2022

Related U.S. Application Data
(60) Provisional application No. 62/970,515, filed on Feb. 5, 2020.

(51) Int. Cl. A61K 31/428; A61K 45/06; A61K 9/00; A61P 27/02

(52) U.S. Cl. CPC A61K 31/428; A61K 45/06; A61K 9/0048; A61P 27/02

(57) ABSTRACT

The present invention provides methods of treating refractive ocular conditions and related complications using compounds of Formula (I) and pharmaceutical compositions thereof either alone or in combination other active agents.

### FIELD OF THE INVENTION

The present invention provides methods of treating refractive ocular disorders such as presbyopia using compounds of Formula (I) and pharmaceutical compositions thereof either alone or in combination with other active agents. The present invention also provides compounds and pharmaceutical compositions.

### BACKGROUND OF THE INVENTION

As a person ages the minimum distance from the eye at which an object will come into focus increases, provided distance vision is corrected or is excellent unaided. This condition of increasing minimum focal length in individuals with excellent corrected or unaided distance vision is called presbyopia.

Presbyopia occurs because as a person ages the eye's accommodative ability which uses near reflex-pupil constriction, convergence of the eyes and particularly ciliary muscle contraction, decreases. This reduction in accommodation results in an inadequate change in the normal thickening and increased curvature of the anterior surface of the lens that is necessary for the shift in focus from distant objects to near objects. Important near focus tasks affected by presbyopia include viewing computer screens and reading print.

Presbyopia may be addressed by the use of eyeglasses, contact lenses or after undergoing invasive surgery. One such optical modification, the monovision procedure, can be executed with the use of glasses, contact lenses or even surgery. The monovision procedure corrects one eye for near focus and the other eye for distance focus. However, monovision correction is normally accompanied by loss of depth perception and distance vision particularly in dim light (e.g. night). Other surgical procedures that have been developed to relieve presbyopia include implantation of intraocular lenses and reshaping of the cornea. Presbyopia may also be addressed with general miotic agents, such as pilocarpine (a non-selective muscarinic acetylcholine receptor agonist), carbachol (a non-selective muscarinic acetylcholine receptor agonist), and phospholine iodide (an acetylcholinesterase inhibitor). These general miotic agents may cause substantial redness, severe nasal congestion and create ciliary muscle spasms.

Thus, there is a need in the art for new, improved, and/or complimentary ocular therapies for conditions such as presbyopia and other refractive related conditions.

### SUMMARY OF THE INVENTION

PCT Publication No. WO 2011/103018 (“WO ‘018’”) describes substituted fused imidazole derivatives that upregulate expression of HMOX1 in vitro. PCT Publication No. WO 2012/094580 (“WO ‘580’”) describes various compounds that modulate cellular oxidative stress including fused imidazole derivatives having a structure similar to or the same as compounds disclosed in WO '018.

The present invention is directed to methods and compositions associated with treatment of one or more refractory ocular disorders including presbyopia.

In certain embodiments of the invention, a compound of the invention is administered alone. In other embodiments, a compound or composition of the invention is administered with one or more other drugs for the treatment of a refractory ocular condition.

The individual treated may be known to have an ocular condition, is suspected of or at risk for having an ocular condition. In embodiments of the invention, an individual is diagnosed with an ocular condition prior to receiving the inventive treatment.

The present invention is also directed to compounds of Formula (I) and pharmaceutically acceptable salts thereof and to pharmaceutical compositions comprising Formula (I) and pharmaceutically acceptable salts thereof, and methods of making thereof. In certain embodiments, the pharmaceutical compositions are suitable for ocular administration.

### BRIEF DESCRIPTION OF THE DRAWINGS

FIG. 1—In vitro effects of Examples 134 and 46 on the change of lens elasticity using the coverslip method in vitro. Data is reported as mean change in lens diameter (μm)±standard error. Graph notations—**, p<0.01; ***, p<0.001 vs no compounds (Dunnett's multiple comparison test).

FIG. 2—In vivo effect of Example 134 on the change of lens elasticity using the coverslip method. Compound administered three times a day. Data is reported as mean change in lens diameter (μm)±standard error. Graph notations—N.S., not significant vs vehicle; **, p<0.01 vs vehicle (R)(Dunnett's multiple comparison test); #, p<0.01 vs vehicle (R)(Student's t-test).

FIG. 3—In vivo effect of Example 46 on the change of lens elasticity using the coverslip method. Compound administered three times a day. Data is reported as mean change in lens diameter (μm)±standard error. Graph notations—N.S., not significant vs vehicle; ***, p<0.001 vs vehicle (R)(Dunnett's multiple comparison test); ###, p<0.001 vs vehicle (R)(Student's t-test).

### DETAILED DESCRIPTION OF INVENTION

### I. Definitions

The following definitions are intended to clarify the terms defined. If a particular term used herein is not specifically defined, the term should not be considered to be indefinite. Rather, such undefined terms are to be construed in accordance with their plain and ordinary meaning to a person of ordinary skill in the field(s) of art to which the invention is directed.

As used herein the term “alkyl” refers to a straight or branched chain saturated hydrocarbon having one to ten carbon atoms, which may be optionally substituted, as herein further described, with multiple degrees of substitution being allowed. Examples of “alkyl” as used herein include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl.

The number carbon atoms in an alkyl group is represented by the phrase “C_{x-y} alkyl,” which refers to an alkyl group, as herein defined, containing from x to y, inclusive, carbon atoms. Thus, C₁₋₆ alkyl represents an alkyl chain having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl.

As used herein, the term “alkylene” refers to a straight or branched chain divalent saturated hydrocarbon radical having from one to ten carbon atoms, which may be optionally substituted as herein further described, with multiple degrees of substitution being allowed. Examples of “alkylene” as used herein include, but are not limited to, methylene, ethylene, n-propylene, 1-methylethylene, 2-methylethylene, dimethylmethylene, n-butylene, 1-methyl-n-propylene, and 2-methyl-n-propylene.

The number of carbon atoms in an alkylene group is represented by the phrase “C_{x-y} alkylene,” which refers to an alkylene group, as herein defined, containing from x to y, inclusive, carbon atoms. Similar terminology will apply for other terms and ranges as well. Thus, C₁₋₄ alkylene represents an alkylene chain having from 1 to 4 carbons atoms, and, for example, includes, but is not limited to, methylene, ethylene, n-propylene, 1-methylethylene, 2-methylethylene, dimethylmethylene, n-butylene, 1-methyl-n-propylene, and 2-methyl-n-propylene.

As used herein, the term “cycloalkyl” refers to a saturated, three- to ten-membered, cyclic hydrocarbon ring, which may be optionally substituted as herein further described, with multiple degrees of substitution being allowed. Such “cycloalkyl” groups are monocyclic, bicyclic, or tricyclic. Examples of “cycloalkyl” groups as used herein include, but are not limited to, cydopropyl, cydobutyl, cydopentyl, cydohexyl, cydoheptyl, norbornyl, and adamantyl.

The number of carbon atoms in a cydoalkyl group will be represented by the phrase “C_{x-y} cydoalkyl,” which refers to a cydoalkyl group, as herein defined, containing from x to y, inclusive, carbon atoms. Similar terminology will apply for other terms and ranges as well. Thus, C₃₋₁₀ cycloalkyl represents a cycloalkyl group having from 3 to 10 carbons as described above, and for example, includes, but is not limited to, cydopropyl, cydobutyl, cydopentyl, cydohexyl, cydoheptyl, norbornyl, and adamantyl.

As used herein, the term “heterocycle” or “heterocyclyl” refers to an optionally substituted mono- or polycyclic saturated ring system containing one or more heteroatoms. Such “hetercycle” or “heterocyclyl” groups may be optionally substituted as herein further described, with multiple degrees of substitution being allowed. The term “heterocycle” or “heterocyclyl,” as used herein, does not include ring systems that contain one or more aromatic rings. Examples of heteroatoms include nitrogen, oxygen, or sulfur atoms, including N-oxides, sulfur oxides, and sulfur dioxides. Typically, the ring is three- to twelve-membered. Such rings may be optionally fused to one or more of another heterocyclic ring(s) or cycloalkyl ring(s). Examples of “heterocyclic” groups, as used herein include, but are not limited to, tetrahydrofuran, tetrahydropyran, 1,4-dioxane, 1,3-dioxane, piperidine, pyrrolidine, morpholine, tetrahydrothiopyran, and tetrahydrothiophene, where attachment can occur at any point on said rings, as long as attachment is chemically feasible. Thus, for example, “morpholine” refers to morpholin-2-yl, morpholin-3-yl, and morpholin-4-yl.

As used herein, when “heterocycle” or “heterocyclyl” is recited as a possible substituent, the “heterocycle” or “heterocyclyl” group can attach through either a carbon atom or any heteroatom, to the extent that attachment at that point is chemically feasible. For example, “heterocyclyl” would include pyrrolidin-1-yl, pyrrolidin-2-yl, and pyrrolidin-3-yl. When “heterocycle” or “heterocyclyl” groups contain a nitrogen atom in the ring, attachment through the nitrogen atom can alternatively be indicated by using an “-ino” suffix with the ring name. For example, pyrrolidino refers to pyrrolidin-1-yl.

As used herein the term “halogen” refers to fluorine, chlorine, bromine, or iodine.

As used herein, the term “oxo” refers to a >C═0 substituent. When an oxo substituent occurs on an otherwise saturated group, such as with an oxo-substituted cycloalkyl group (e.g., 3-oxo-cyclobutyl), the substituted group is still intended to be a saturated group.

As used herein, the term “heteroaryl” refers to a five- to fourteen-membered optionally substituted mono- or polycyclic ring system, which contains at least one aromatic ring and also contains one or more heteroatoms. Such “heteroaryl” groups may be optionally substituted as herein further described, with multiple degrees of substitution being allowed. In a polycyclic “heteroaryl” group that contains at least one aromatic ring and at least one non-aromatic ring, the aromatic ring(s) need not contain a heteroatom. Thus, for example, “heteroaryl,” as used herein, would include indolinyl. Further, the point of attachment may be to any ring within the ring system without regard to whether the ring containing the attachment point is aromatic or contains a heteroatom. Thus, for example, “heteroaryl,” as used herein, would include indolin-1-yl, indolin-3-yl, and indolin-5-yl. Examples of heteroatoms include nitrogen, oxygen, or sulfur atoms, including N-oxides, sulfur oxides, and sulfur dioxides, where feasible. Examples of “heteraryl” groups, as used herein include, but are not limited to, furyl, thiophenyl, pyrrolyl, oxazolyl, thiazolyl, imidazolyl, isoxazolyl, isothiazolyl, 1,2,4-triazolyl, pyrazolyl, pyridinyl, pyridazinyl, pyrimidinyl, indolyl, isoindolyl, benzo[b]thiophenyl, benzimidazolyl, benzothiazolyl, pteridinyl, and phenazinyl, where attachment can occur at any point on said rings, as long as attachment is chemically feasible. Thus, for example, “thiazolyl” refers to thiazol-2-yl, thiazol-4-yl, and thiaz-5-yl.

As used herein, when “heteroaryl” is recited as a possible substituent, the “heteroaryl” group can attach through either a carbon atom or any heteroatom, to the extent that attachment at that point is chemically feasible.

As used herein, the term “heterocyclylene” refers to an optionally substituted bivalent heterocyclyl group (as defined above). The points of attachment may be to the same ring atom or to different ring atoms, as long as attachment is chemically feasible. The two points of attachment can each independently be to either a carbon atom or a heteroatom, as long as attachment is chemically feasible. Examples include, but are not limited to

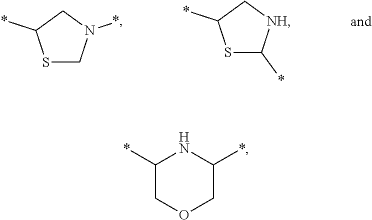

where the asterisks indicate points of attachment.

As used herein, the term “heteroarylene” refers to an optionally substituted bivalent heteroaryl group (as defined above). The points of attachment may be to the same ring atom or to different ring atoms, as long as attachment is chemically feasible. The two points of attachment can each independently be to either a carbon atom or a heteroatom, as long as attachment is chemically feasible. Examples include, but are not limited to,

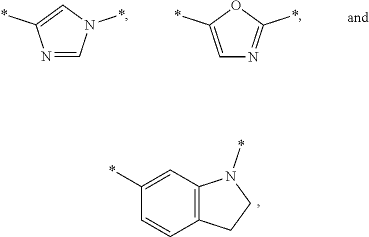

where the asterisks indicate points of attachment.

Various other chemical terms or abbreviations have their standard meaning to the skilled artisan. For example: “hydroxyl” refers to —OH; “methoxy” refers to —OCH₃; “cyano” refers to —CN; “amino” refers to —NH₂; “methylamino” refers to —NHCH₃; “sulfonyl” refers to —SO₂—; “carbonyl” refers to —C(O)—; “carboxy” or “carboxyl” refer to —CO₂H, and the like. Further, when a name recited multiple moieties, e.g., “methylaminocarbonyl-methyl”, an earlier-recited moiety is further from the point of attachment than any later-recited moieties. Thus, a term such as “methylaminocarbonylmethyl” refers to —CH₂—C(O)—NH—CH₃.

As used herein, the term “substituted” refers to substitution of one or more hydrogens of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week, or a compound which maintains its integrity long enough to be useful for therapeutic or prophylactic administration to a subject. As used herein, the phrases “substituted with one or more . . . ” or “substituted one or more times . . . ” refer to a number of substituents that equals from one to the maximum number of substituents possible based on the number of available bonding sites, provided that the above conditions of stability and chemical feasibility are met.

As used herein, the various functional groups represented will be understood to have a point of attachment at the functional group having the hyphen or dash (-) or an asterisk (*). In other words, in the case of —CH₂CH₂CH₃, it will be understood that the point of attachment is the CH₂ group at the far left. If a group is recited without an asterisk or a dash, then the attachment point is indicated by the plain and ordinary meaning of the recited group.

When any variable occurs more than one time in any one constituent (e.g., R^{d}), or multiple constituents, its definition on each occurrence is independent of its definition on every other occurrence.

As used herein, multi-atom bivalent species are to be read from left to right. For example, if the specification or claims recite A-D-E and D is defined as —OC(O)—, the resulting group with D replaced is: A-OC(O)-E and not A-C(O)O-E.

As used herein, the term “optionally” means that the subsequently described event(s) may or may not occur.

As used herein, “administer” or “administering” means to introduce, such as to introduce to a subject a compound or composition. The term is not limited to any specific mode of delivery, and can include, for example, intravenous delivery, transdermal delivery, oral delivery, nasal delivery, ocular delivery and rectal delivery. Furthermore, depending on the mode of delivery, the administering can be carried out by various individuals, including, for example, a health-care professional (e.g., physician, nurse, etc.), a pharmacist, or the subject (i.e., self-administration).

As used herein, “treat” or “treating” or “treatment” can refer to one or more of delaying the progress of a disease or condition, controlling a disease or condition, delaying the onset of a disease or condition, ameliorating one or more symptoms characteristic of a disease or condition, or delaying the recurrence of a disease or condition or characteristic symptoms thereof, depending on the nature of a disease or condition and its characteristic symptoms. “Treat” or “treating” or “treatment” may also refers to inhibiting the disease, either physically, (e.g., stabilization of a discernible symptom), physiologically, (e.g., stabilization of a physical parameter), or both, and to inhibiting at least one physical parameter that may or may not be discernible to the subject. In certain embodiments, “treat” or “treating” or “treatment” refers to delaying the onset of the disease or at least one or more symptoms thereof in a subject which may be exposed to or predisposed to a disease even though that subject does not yet experience or display symptoms of the disease.

As used herein, “subject” may refer any mammal such as, but not limited to, humans. In one embodiment, the subject is a human. In another embodiment, the host is a human who exhibits one or more symptoms characteristic of a refractive ocular condition. The term “subject” does not require one to have any particular status with respect to any hospital, clinic, or research facility (e.g., as an admitted patient, a study participant, or the like). In an embodiment, the subject may be “a subject in need thereof.”

“Therapeutically effective amount” refers to the amount of a compound that, when administered to a subject for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the subject to be treated, and the judgment of the prescribing physician. An appropriate amount in any given instance may be ascertained by those skilled in the art or capable of determination by routine experimentation. For example, a therapeutically effective amount may be determined by measuring clinical outcomes including, but not limited to, the elasticity, stiffness, viscosity, density, or opacity of a lens or by measuring a subject's visual acuity. In certain embodiments, a therapeutically effective amount of compound of Formula (I) or a pharmaceutically acceptable salt thereof used in the methods and compositions of the present invention may be an amount that is capable of reducing, reversing, and/or slowing the rate of oxidative damage of a lens.

As used herein, the term “compound of the invention” includes free acids, free bases, and any salts thereof of the compound of Formula (I). Thus, phrases such as “compound of embodiment 1” or “compound of claim 1” refer to any free acids, free bases, and any salts thereof that are encompassed by embodiment 1 or claim 1, respectively.

### II. Methods of Treatment

A. Treatment of an Ocular Condition and Related Disorders with Compounds of the Invention

In certain embodiments, the present invention is directed to methods for the treatment of a refractory ocular condition comprising administering a compound of Formula (I) or a pharmaceutically acceptable salt thereof to a subject in need thereof.

In certain embodiments, the present invention is directed to compositions and methods for the treatment of a refractory ocular condition comprising administering a pharmaceutical composition comprising a compound of Formula (I) or a pharmaceutically acceptable salt thereof. In certain embodiments, the pharmaceutical formulation is suitable for ocular administration. In further embodiments, the pharmaceutical formulation is administered ocularly and comprises a concentration from about 0.01% to about 5.0% w/v of a compound of Formula (I) or a pharmaceutically acceptable salt thereof, or a concentration from about 0.10% to about 3.0% w/v of a compound of Formula (I) or a pharmaceutically acceptable salt thereof.

In certain other embodiments, the present invention is directed to a method of treating presbyopia comprising administering to a subject in need thereof a compound or a pharmaceutical composition of the present invention.

In certain other embodiments, the present invention is directed to a method of treating presbyopia comprising administering to a subject in need thereof a compound or a pharmaceutical composition of the present invention, wherein near vision acuity of the subject is improved by about 3 lines of resolution or more for at least 6 hours.

In certain other embodiments, the present invention is directed to a method of treating presbyopia comprising administering to a subject in need thereof a compound or a pharmaceutical composition of the present invention, wherein near vision acuity of the subject is improved to 20/40 near vision or wherein the subject has a gain of at least 10 letters in distance-corrected near visual acuity in the nondominant eye and/or as bilateral vision relative to vision prior to treatment.

In certain other embodiments, the present invention is directed to method of treating a refractive error of the eye in a subject in need thereof comprising administering to a subject in need thereof a compound or a pharmaceutical composition of the present invention wherein the refractive error of the eye is selected from presbyopia, myopia, hyperopia, astigmatism or a combination thereof.

In certain other embodiments, the present invention is further directed to a method for treating a refractive error of the eye comprising administering to a subject in need thereof a compound or a pharmaceutical composition of the present invention, wherein the size of the pupil is reduced to from about 1.5 to about 2.5 millimeters, or from about 1.7 to about 2.2 millimeters and wherein the refractive error is selected from the group consisting of a range of distance corrected vision for mild to moderate hyperopia of 3.0 diopters (D) or less; mild to moderate myopia of −5.0 D or less; regular astigmatism of 3.0 D or less; uncorrected distance vision for emmetropes of +0.50 to −0.50 spherical equivalent with regular astigmatism of 0.75 D or less; corneal irregular astigmatism, an ectasia induced corneal irregularity, a pellucid induced corneal irregularity, a higher order aberration and a refractive surgery induced higher order aberration.

The present invention is further directed to a method of increasing the visual depth of field (i.e. depth of focus) secondary to pupil miosis, comprising administering to a subject in need thereof a compound or pharmaceutical composition of the present invention.

The present invention is further directed to a method of treating a refractory ocular condition such as presbyopia comprising administering to a subject in need thereof a compound or a pharmaceutical composition of the present invention, wherein following treatment the subject is able to identify correctly at least 2, 5, or 10 additional letters in distance-corrected near visual acuity (DCNVA) in the nondominant eye or in bilateral vision relative to baseline (i.e., prior to treatment).

The present invention is further directed to a method of treating a refractory ocular condition such as presbyopia comprising administering to a subject in need thereof a compound or a pharmaceutical composition of the present invention, wherein following treatment the subject is able to achieve or correctly identify at least 60, 65, 70, 75, or 80 or more ETDRS letters in binocular DCNVA.

The present invention is further directed to a method of treating a refractory ocular condition such as presbyopia comprising administering to a subject in need thereof a compound or a pharmaceutical composition of the present invention, wherein a subject has at least a 5%, 10%, 15%, 20%, or 25% increase or improvement in binocular DCNVA relative to baseline (i.e., prior to treatment).

The present invention is further directed to a method of treating a refractory ocular condition such as presbyopia comprising administering to a subject in need thereof a compound or a pharmaceutical composition of the present invention, wherein following treatment the subject has a decrease (improvement) in Log MAR score of at least 0.050, 0.075, 0.100, 0.125, 0.150, 0.175, or 0.200 relative to baseline (i.e., prior to treatment). In another embodiment, the subject's Log MAR score is less than 0.300 Log MAR, or less than 0.250 Log MAR, or is less than 0.225 Log MAR, or less than 0.200 Log MAR following treatment.

The test for visual acuity may use the Snellen chart, the Early Treatment for Diabetic Retinopathy Study (ETDRS) chart, or another chart.

The present invention is further directed to a method of allowing binocular physiologic topical presbyopic correction.

The present invention is further directed to a method of eliminating the need for monocular limitation due to distance blur, or reduced to treatment of mild hyperopes to counteract induced myopic blur, as typically associated with pilocarpine, or pilocarpine and alpha agonist combinations.

The present invention is further directed to a method of a refractory ocular condition by inhibiting or reversing the progression of age related degeneration of a crystalline lens in an eye, comprising administering to a subject in need thereof a compound or a pharmaceutical composition of the present invention.

The present invention is further directed at eliminating optical aberrations induced by corneal irregularity, opacities, or very high degrees of regular astigmatism that include regions adjacent or peripheral to the central 1.5 mm optical zone, and thereby inducing improved visual acuity and quality of vision by filtering out these aberrant optics in those suffering from irregular astigmatism or high degrees of more regular astigmatism, such as occurs in conditions such as keratoconus, photorefractive keratectomy induced corneal haze, diffuse lamellar keratitis (“DLK”) (post-lasik DLK), other iatrogenic corneal induced irregularity such as cataract incision, glaucoma filtering blebs, implanted glaucoma valves, corneal inlays with or without removal, ectasia post corneal surgery (lasik), and secondary to infection.

The present invention is also directed to methods of treating irregular astigmatism, keratoconic ectasia, and low myopia, or hyperopia, with or without astigmatism, comprising administering to a subject in need thereof a compound or a pharmaceutical composition of the present invention.

The compounds and pharmaceutical composition described herein may also be used in a method for treating, reducing, or preventing oxidation damage or stress to cells, including ocular cells or the lens of an eye. Such a method includes the step of administering a compound of Formula (I) or a pharmaceutically acceptable salt thereof or a pharmaceutical composition comprising Formula (I) or a pharmaceutically acceptable salt thereof to such a cell, either in vitro or in vivo. In one embodiment, a compound or pharmaceutical composition of the invention is delivered to a lens, either in vitro or in vivo.

Lens elasticity decreases with age and is a primary diagnostic and causative factor for presbyopia. Lens elasticity can be measured as accommodative amplitude in diopters (D). The lower the value of D, the less elastic the lens. In one embodiment, the agents described herein may decrease and/or maintain D at a value that is greater than the D value exhibited by an untreated lens of about the same age. In one embodiment, D is increased and/or maintained at a value about 2, 5, 7, 10, 15, 25, 50, 100, 150, or 200 percent above the line. In another embodiment, the agents described herein may cause a reduction in the rate of decline of elasticity (i.e., reduction in the rate of decrease in diopters) for an individual lens compared to the elasticity of the same lens before treatment. In another embodiment, the methods provide an objective increase in elasticity of at least about 0.1, 0.2, 0.5, 1, 1.2, 1.5, 1.8, 2, 2.5, 3, or 5 diopters.

Therapeutic efficacy can also be measured in terms of lens opacity. Lens opacity increases with age and may be used as an indirect diagnostic for lens elasticity. In one embodiment, the agents described herein may decrease and/or maintain lens opacity at a value that is less than the opacity value exhibited by an untreated lens of about the same age.

Treatment methods can be performed on subjects of any age, such as subjects that are 20, 25, 30, 35, 40, 45, 50, 52, 55, 57, 60, 70, 75, or 80 years of age or older, or between the ages of 20-40, or 30-50, or 40-60, or 50-70, or 60-80, or 70-90 years of age.

In certain embodiments, prior to treatment the subjects may have impaired near vision in each eye and/or when using both eyes, without any near correction. In other embodiments, prior to treatment, the subject may need a certain level of near correction. In other embodiments, prior to treatment, the subject has Distance Corrected Near Visual Acuity of 20/40 or worse than 20/40, 20/50, 20/70, or 20/100 and/or best Corrected Distance Visual Acuity of 20/40, or 20/20 or better than 20/20 in at least one eye or in both eyes. In other embodiments, the subject may not have or suffer from significant astigmatism, glaucoma, diabetes, diabetic retinopathy, or cataracts. In other embodiments, the subject may not have had eye surgery or ocular trauma.

Treatment methods include frequency of administration of every 6 hours, every 8 hours, 12 hours, 48 hours, 72 hours, 96 hours, 1 week, or 2 weeks. Treatment methods also include frequency of administration of four times a day (QID), three times a day (TID), two times a day (BID), or once a day (QD).

In another embodiment, the invention provides a method of treatment comprising administering a compound (or salt) of any one of embodiments 1 to 250 or of any one of Examples 1-474 (or salt) to a subject. In another embodiment, the invention provides a method of treatment comprising administering between 0.01 milligrams and 2 grams of a compound (or salt) of any one of embodiments 1 to 250 or of any one of Examples 1-474 (or salt) to a subject.

In each of the methods described above or below, a compound (or salt) of any of embodiments 1 to 250 or of any one of Examples 1-474 (or salt) may be administered to a subject as part of a pharmaceutically formulation, as described herein.

In each of the methods described herein, the method may further include the step of determining whether the subject has a refractive ocular condition.

B. Combination Treatments

Methods for treating refractive ocular conditions described herein may also include administering a compound of the invention in combination with or alternation with another active agent known to be useful in ocular disease, or with adjunctive agents that may not be effective alone, but may contribute to the efficacy of the active agent. For example, adjunctive agents might include one or more amino acids or choline to enhance the efficacy of the active agent.

The term “co-administer” means to administer more than one active agent, such that the duration of physiological effect of one active agent overlaps with the physiological effect of a second active agent. In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In an embodiment, the additional active agent is selected from the group consisting of general miotic agents, such as a non-selective muscarinic acetylcholine receptor agonist (pilocarpine), a non-selective muscarinic acetylcholine receptor agonist (carbachol), an acetylcholinesterase inhibitor (phospholine iodide), or an alpha 2 selective vasoconstrictor (brimonidine). In other embodiments, the additional active agent is selected from the group consisting of lipoic acid, lipoic acid derivatives (such as lipoic acid choline ester), Nrf2 activators, antioxidants, detoxification agents, and anti-inflammatory agents. In one embodiment, the invention provides a pharmaceutical composition comprising a compound (or salt) of any one of embodiments 1 to 250 (or salt) or of any one of Examples 1-474 (or salt) and at least one other active ingredient selected from lipoic acid, lipoic acid derivatives (such as lipoic acid choline ester), Nrf2 activators, antioxidants, detoxification agents, and anti-inflammatory agents. In another embodiment, the invention provides for the use of a compound (or salt) of any one of embodiments 1 to 250 or of any one of Examples 1-474 (or salt) in combination with at least one other active ingredient selected from lipoic acid, lipoic acid derivatives (such as lipoic acid choline ester), Nrf2 activators, antioxidants, detoxification agents, and anti-inflammatory agents for simultaneous, subsequent, or sequential administration.

### III. Compositions for Use in Treating Refractive Ocular Conditions

A. Compounds of the Invention (Compounds of Formula (I)))

A compound of Formula (I) has the structure shown below

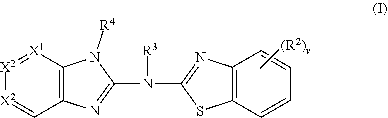

wherein
X¹ is ═N— or ═CH—;
X² is ═C(R¹)— and X³ is ═C(-L-G)-; or X² is ═C(-L-G)- and X³ is ═C(R¹)—;
G is hydrogen, —C₁₋₈ alkyl, —C₃₋₁₀ cycloalkyl, —C₁₋₆ alkylene-C₃₋₁₀ cycloaklyl, heterocyclyl, —C₁₋₆ alkylene-C₃₋₁₀ heterocyclyl, phenyl, heteroaryl, or NR^{h}R^{k}, where the alkyl, alkylene, cycloalkyl, heterocyclyl, phenyl, and heteroaryl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

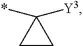

where Y³ is cyclopropyl, —CF₃, —OCF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, heteroarylene optionally substituted one or more times with substituents independently selected from R^{x}, or heterocyclylene optionally substituted one or more times with substituents independently selected from R^{x}; or the group -L-G is -cyano;
R¹ is hydrogen, R^{a}, phenyl, or heteroaryl, where the phenyl and heteroaryl groups are optionally substituted one or more times with substituents independently selected from R^{x};
R² is R^{b};
R³ is hydrogen, —C₁₋₆ alkyl, or —C₁₋₆ alkylene-C₃₋₁₀ cycloaklyl, where the alkyl, alkylene, and cycloalkyl groups are optionally substituted one or more times with substituents independently selected from R^{z};
R⁴ is —C₁₋₆ alkyl or —C₁₋₆ alkylene-C₃₋₁₀ cycloaklyl, where the alkyl, alkylene, and cycloalkyl groups are optionally substituted one or more times with substituents independently selected from R^{y};
R⁶ is hydrogen, —C₁₋₆ alkyl, —C₁₋₆ alkylene-C₃₋₁₀ cycloaklyl, where the alkyl, alkylene, and cycloalkyl groups are optionally substituted one or more times with substituents independently selected from R^{x};
R^{a} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{d},
   i) —S(O)_{w}—R^{d},
   j) —S(O)₂O—R^{d},
   k) —NR^{d}R^{e},
   l) —C(O)—R^{d},
   m) —C(O)—O—R^{d},
   n) —OC(O)—R^{d},
   o) —C(O)NR^{d}R^{e},
   p) —C(O)-heterocyclyl,
   a) —NR^{d}C(O)R^{e},
   r) —OC(O)NR^{d}R^{e},
   s) —NR^{d}C(O)OR^{d}, or
   t) —NR^{d}C(O)NR^{d}R^{e},
   where the alkyl, cycloalkyl, and heterocyclyl groups are optionally substituted one or more times with substituents independently selected from R^{y};
R^{b} is
   a) -halogen,
   b) —C₁₋₆ alkyl,
   c) —C₃₋₁₀ cycloalkyl,
   d) -heterocyclyl,
   e) -phenyl,
   f) -heteroaryl,
   g) -cyano,
   h) —CF₃,
   i) —OCF₃,
   j) —O—R^{f},
   k) —S(O)_{w}—R^{f},
   l) —S(O)₂O—R^{f},
   m) —NR^{f}R^{g},
   n) —C(O)—R^{f},
   o) —C(O)—O—R^{f},
   p) —OC(O)—R^{f},
   a) —C(O)NR^{f}R^{g},
   r) —C(O)-heterocyclyl,
   s) —NR^{f}C(O)R^{g},
   t) —OC(O)NR^{f}R^{g},
   u) —NR^{f}C(O)OR^{f}, or
   v) —NR^{f}C(O)NR^{f}R^{g},
   where the alkyl, cycloalkyl, heterocyclyl, phenyl, and heteroaryl groups are optionally substituted one or more times with substituents independently selected from R^{z};
R^{c} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{h},
   i) —S(O)_{w}—R^{h},
   j) —S(O)₂O—R^{h},
   k) —NR^{h}R^{k},
   l) —C(O)—R^{h},
   m) —C(O)—O—R^{h},
   n) —OC(O)—R^{h},
   o) —C(O)NR^{h}R^{k},
   p) —C(O)-heterocyclyl,
   a) —NR^{h}C(O)R^{k},
   r) —OC(O)NR^{h}R^{k},
   s) —NR^{h}C(O)OR^{k},
   t) —NR^{h}C(O)NR^{h}R^{k},
   u) —NR^{h}S(O)_{w}R^{k},
   v) -phenyl,
   w) -heteroaryl, or
   x) —O—(C₁₋₄ alkylene)-O—(C₁₋₄ alkylene)-N(R^{h})C(O)—OR^{k},
   where the alkylene, alkyl, cycloalkyl, heterocyclyl, phenyl, and heteroaryl groups are optionally substituted one or more times with substituents independently selected from R^{x};
R^{d} and R^{e} are independently hydrogen, C₁₋₆ alkyl, or C₃₋₁₀ cycloalkyl, where the alkyl and cycloalkyl groups are optionally substituted one or more times with substituents independently selected from R^{y}; or, if R^{d} and R^{e} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{y};
R^{f} and R^{g} are independently hydrogen, C₁₋₆ alkyl, C₃₋₁₀ cycloalkyl, phenyl, or heteroaryl, where the alkyl, cycloalkyl, phenyl, and heteroaryl groups are optionally substituted one or more times with substituents independently selected from R^{z}; or, if R^{f} and R^{g} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{z};
R^{h} and R^{k} are independently hydrogen, C₁₋₆ alkyl, C₃₋₁₀ cycloalkyl, heterocyclyl, phenyl, or heteroaryl, where the alkyl, cycloalkyl, heterocyclyl, phenyl, and heteroaryl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x};
R^{y} is
   a) -halogen,
   b) —NH₂,
   c) -cyano,
   d) -carboxy,
   e) -hydroxy,
   f) -thiol,
   g) —CF₃,
   h) —OCF₃,
   i) —C(O)—NH₂,
   j) —S(O)₂—NH₂,
   k) oxo,
   l) —C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   m) -heterocyclyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   n) —C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   o) —O—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   p) —O—C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   q) —NH—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   r) —N(C₁₋₆ alkyl)₂ optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   s) —C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   t) —C(O)—O—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   u) —S—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   v) —S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   w) —C(O)—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   x) —C(O)—N(C₁₋₆ alkyl)₂, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   y) —S(O)₂—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   z) —S(O)₂—N(C₁₋₆ alkyl)₂, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   aa) —NH—C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂, or
   bb) —NH—S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂;
R^{x} is
   a) —R^{y}
   b) -phenyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   c) -heteroaryl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   d) —O-phenyl,
   e) —O-heteroaryl,
   f) —C(O)-phenyl,
   g) —C(O)-heteroaryl,
   h) —C(O)—O-phenyl, or
   i) —C(O)—O-heteroaryl;
R^{z} is
   a) —R^{y}
   b) -phenyl,
   c) -heteroaryl;
   d) —O-phenyl,
   e) —O-heteroaryl,
   f) —C(O)-phenyl,
   g) —C(O)-heteroaryl,
   h) —C(O)—O-phenyl, or
   i) —C(O)—O-heteroaryl;
v is an integer from 0 to 4, and
w is an integer from 0 to 2.
   Embodiment 2: A compound according to embodiment 1 wherein
G is hydrogen, —C₁₋₈ alkyl, —C₃₋₁₀ cycloalkyl, —C₁₋₆ alkylene-C₃₋₁₀ cycloaklyl, heterocyclyl, phenyl, heteroaryl, or NR^{h}R^{k}, where the alkyl, alkylene, cycloalkyl, heterocyclyl, phenyl, and heteroaryl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

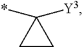

where Y³ is -cyclopropyl, —CF₃, —OCF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
R^{c} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{h},
   l) —S(O)_{w}—R^{h},
   j) —S(O)₂O—R^{h},
   k) —NR^{h}R^{k},
   l) —C(O)—R^{h},
   m) —C(O)—O—R^{h},
   n) —OC(O)—R^{h},
   o) —C(O)NR^{h}R^{k},
   p) —C(O)-heterocyclyl,
   a) —NR^{h}C(O)R^{k},
   r) —OC(O)NR^{h}R^{k},
   s) —NR^{h}C(O)OR^{k},
   t) —NR^{h}C(O)NR^{h}R^{k},
   u) -phenyl,
   v) -heteroaryl, or
   w) —O—(C₁₋₄ alkylene)-O—(C₁₋₄ alkylene)-N(R^{h})C(O)—OR^{k},
   where the alkylene, alkyl, cycloalkyl, heterocyclyl, phenyl, and heteroaryl groups are optionally substituted one or more times with substituents independently selected from R^{x};
R^{h} and R^{k} are independently hydrogen, C₁₋₆ alkyl, C₃₋₁₀ cycloalkyl, phenyl, or heteroaryl, where the alkyl, cycloalkyl, phenyl, and heteroaryl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x}; and
R^{y} is
   a) -halogen,
   b) —NH₂,
   c) -cyano,
   d) -carboxy,
   e) —C₁₋₆ alkyl, optionally substituted one or more times with halogen,
   f) -heterocyclyl, optionally substituted one or more times with halogen,
   g) —C₃₋₁₀ cycloalkyl, optionally substituted one or more times with halogen,
   h) —O—C₁₋₆ alkyl, optionally substituted one or more times with halogen,
   i) —O—C₃₋₁₀ cycloalkyl, optionally substituted one or more times with halogen,
   j) -hydroxy,
   k) -thiol,
   l) —CF₃,
   m) —OCF₃,
   n) —C(O)—C₁₋₆ alkyl, optionally substituted one or more times with halogen,
   o) —C(O)—O—C₁₋₆ alkyl, optionally substituted one or more times with halogen,
   p) —S—C₁₋₆ alkyl, optionally substituted one or more times with halogen, or
   q) —S(O)₂—C₁₋₆ alkyl, optionally substituted one or more times with halogen.
      Embodiment 3: A compound according to embodiment 2, wherein
   R³ is hydrogen.
      Embodiment 4: A compound according to embodiment 2, wherein
   R³ is methyl.
      Embodiment 5: A compound according to any one of embodiments 2 to 4, wherein
   X¹ is ═N—.
      Embodiment 6: A compound according to any one of embodiments 2 to 4, wherein
   X¹ is ═CH—.
      Embodiment 7: A compound according to any one of embodiments 2 to 6, wherein
   v is an integer from 0 to 2.
      Embodiment 8: A compound according to any one of embodiments 2 to 6, wherein
   v is 0 or 1.
      Embodiment 9: A compound according to any one of embodiments 2 to 6, wherein
   v is 1.
      Embodiment 10: A compound according to any one of embodiments 2 to 6, wherein
   v is 1, and R² is attached at either the 5-position or the 6-position of the benzothiazole ring.
      Embodiment 11: A compound according to any one of embodiments 2 to 6, wherein
   v is 1, and R² is attached at the 6-position of the benzothiazole ring.
      Embodiment 12: A compound according to any one of embodiments 2 to 68, wherein
   v is 2, and one R² is attached at the 6-position of the benzothiazole ring.
      Embodiment 13: A compound according to any one of embodiments 2 to 6, wherein
   v is 2, and R² is attached at the 5-position and the 6-position of the benzothiazole ring.
      Embodiment 14: A compound according to any one of embodiments 2 to 13, wherein
   R² is -halogen, —C₁₋₆ alkyl, —CF₃, —OCF₃, —O—R^{f}, or —S(O)_{w}—R^{f}, where the alkyl group is optionally substituted one or more times with substituents independently selected from R^{z}.
      Embodiment 15: A compound according to any one of embodiments 2 to 13, wherein
   R² is -halogen, -methyl, —CF₃, —OCF₃, —SCF₃, —O-heteroaryl, or —S(O)₂—CH₃.
      Embodiment 16: A compound according to any one of embodiments 2 to 13, wherein
   R² is selected from —Cl, —F, —CF₃, and —OCF₃.
      Embodiment 17: A compound according to any one of embodiments 2 to 13, wherein
   R² is —OCF₃.
      Embodiment 18: A compound according to any one of embodiments 2 to 13, wherein
   R² is —CF₃.
      Embodiment 19: A compound according to any one of embodiments 2 to 13, wherein
   R² is —F.
      Embodiment 20: A compound according to any one of embodiments 2 to 13, wherein
   R² is —Cl.
      Embodiment 21: A compound according to any one of embodiments 2 to 20, wherein
   R⁴ is -methyl, -ethyl, -n-propyl, -isopropyl, -n-butyl, -sec-butyl, -isobutyl, -tert-butyl, —(CH₂)₁₋₂—OCH₃, —(CH₂)₁₋₂—F, —(CH₂)₁₋₂—C₁, —(CH₂)₁₋₂—OCF₃, —(CH₂)₁₋₂—NH₂, —(CH₂)₁₋₂—CN, —(CH₂)₁₋₂—OH, —(CH₂)₁₋₂—CF₃, —(CH₂)₁₋₂—CO₂H, —(CH₂)₁₋₂—SH, —(CH₂)₁₋₂—SCH₃, —(CH₂)₁₋₂—S(O)₂CH₃, —(CH₂)₁₋₂—OCH₂CH₃, —(CH₂)₁₋₂—SCH₂CH₃, —(CH₂)₁₋₂—S(O)₂CH₂CH₃, —(CH₂)₁₋₂—NH—CH₃, or —(CH₂)₁₋₂—N(CH₃)₂.
      Embodiment 22: A compound according to any one of embodiments 2 to 21, wherein
   R⁴ is -methyl, -ethyl, -isopropyl, -isobutyl, —CH₂CH₂—OCH₃, —CH₂CH₂—F, or —CH₂CH₂—NH₂.
      Embodiment 23: A compound according to any one of embodiments 2 to 22, wherein
   R⁴ is -methyl, -ethyl, -isopropyl, or -isobutyl.
      Embodiment 24: A compound according to any one of embodiments 2 to 23, wherein
   R⁴ is -methyl.
      Embodiment 25: A compound according to any one of embodiments 2 to 23, wherein
   R⁴ is -ethyl.
      Embodiment 26: A compound according to any one of embodiments 2 to 21, wherein
   R⁴ is —(CH₂)₂—OCH₃, —(CH₂)₂—F, —(CH₂)₂—Cl, —(CH₂)₂—OCF₃, —(CH₂)₂—NH₂, —(CH₂)₂—CN, —(CH₂)₂—OH, —(CH₂)₂—CF₃, —(CH₂)₂—CO₂H, —(CH₂)₂—SH, —(CH₂)₂—SCH₃, or —(CH₂)₂—S(O)₂CH₃.
      Embodiment 27: A compound according to any one of embodiments 2 to 26, wherein
   R¹ is selected from hydrogen, —OCH₃, —F, —Cl, —NH₂, -cyano, —OH, —CF₃, —OCF₃, —SH, —S—C₁₋₆ alkyl, —S(O)₂—C₁₋₆ alkyl, —CO₂H, —NH—C₁₋₆ alkyl, —N(C₁₋₆ alkyl)₂, and —NH—C₁₋₆ alkyl.
      Embodiment 28: A compound according to any one of embodiments 2 to 26, wherein
   R¹ is selected from —OCH₃, —F, —CF₃, —OCF₃, —N(CH₃)₂, —N(CH₂CH₃)₂, and —N(CH₃)(CH₂CH₃).
      Embodiment 29: A compound according to any one of embodiments 2 to 26, wherein
   R¹ is selected from hydrogen, —OCH₃, and —F.
      Embodiment 30: A compound according to any one of embodiments 2 to 26, wherein
   R¹ is hydrogen.
      Embodiment 31: A compound according to any one of embodiments 2 to 30, wherein
G is hydrogen, —C₁₋₆ alkyl, —C₃₋₁₀ cycloalkyl, —C₁₋₆ alkylene-C₃₋₈ cycloaklyl, heterocyclyl, or NR^{h}R^{k}, where the alkyl, alkylene, cycloalkyl, and heterocyclyl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

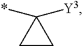

where Y³ is -cyclopropyl, —CF₃, —OCF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, or heterocyclylene optionally substituted one or more times with substituents independently selected from R^{x}; or the group -L-G is -cyano;
R¹ is hydrogen or R^{a};
R^{e} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{h},
   l) —S(O)_{w}—R^{h},
   j) —S(O)₂O—R^{h},
   k) —NR^{h}R^{k},
   l) —C(O)—R^{h},
   m) —C(O)—O—R^{h},
   n) —OC(O)—R^{h},
   o) —C(O)NR^{h}R^{k},
   p) —C(O)-heterocyclyl,
   a) —NR^{h}C(O)R^{k},
   r) —OC(O)NR^{h}R^{k},
   s) —NR^{h}C(O)OR^{k},
   t) —NR^{h}C(O)NR^{h}R^{k}, or
   u) —O—(C₁₋₄ alkylene)-O—(C₁₋₄ alkylene)-N(R^{h})C(O)—OR^{k},
   where the alkylene, alkyl, cycloalkyl, and heterocyclyl groups are optionally substituted one or more times with substituents independently selected from R^{x};
R^{h} and R^{k} are independently hydrogen, C₁₋₆ alkyl, or C₃₋₁₀ cycloalkyl, where the alkyl, and cycloalkyl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x}; and
R^{x} is R^{y}.
   Embodiment 32: A compound according to any one of embodiments 2 to 31, wherein
   -L-G is not -cyano.
      Embodiment 33: A compound according to any one of embodiments 2 to 32, wherein
   -L-G is —C(O)NR^{h}R^{k}.
      Embodiment 34: A compound according to any one of embodiments 2 to 32, wherein
   L is —C(O)N(R⁶)— or —C(O)—O—.
      Embodiment 35: A compound according to any one of embodiments 2 to 32, wherein
   L is —C(O)N(R⁶)—.
      Embodiment 36: A compound according to any one of embodiments 2 to 32, wherein
   L is not —CH₂—C(O)N(R⁶)—.
      Embodiment 37: A compound according to any one of embodiments 2 to 32, wherein
   L is —C(O)—O—.
      Embodiment 38: A compound according to any one of embodiments 2 to 32, wherein
   L is —C(O)—.
      Embodiment 39: A compound according to any one of embodiments 2 to 32, wherein
   L is —S(O)₂—.
      Embodiment 40: A compound according to any one of embodiments 2 to 30, wherein
   L is heteroarylene optionally substituted one or more times with substituents independently selected from R^{x}.
      Embodiment 41: A compound according to any one of embodiments 2 to 40, wherein
   R⁶ is hydrogen.
      Embodiment 42: A compound according to any one of embodiments 2 to 40, wherein
   R⁶ is hydrogen or -methyl.
      Embodiment 43: A compound according to any one of embodiments 2 to 42, wherein
   G is hydrogen, —C₁₋₈ alkyl, —C₃₋₁₀ cycloalkyl, or —C₁₋₆ alkylene-C₃₋₈ cycloaklyl, where the alkyl, cycloalkyl, and alkylene groups are optionally substituted one or more times with substituents independently selected from R^{x}.
      Embodiment 44: A compound according to any one of embodiments 2 to 42, wherein
   G is —H, -methyl, -ethyl, -n-propyl, -isopropyl, -isobutyl, —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, or —C(Y³)(CH₃)₂, where Y³ is -cyclopropyl, —CF₃, —OCF₃, —OCH₃, —OCH₂CH₃, —F, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, —NH—C(O)—CH₃, —NH—C(O)—CH₂CH₃, or C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, or —N(CH₂CH₃)₂.
      Embodiment 45: A compound according to any one of embodiments 2 to 42, wherein
   G is -methyl, -ethyl, -n-propyl, -isopropyl, or -isobutyl, where each is optionally substituted one or more times with substituents independently selected from —CF₃, —OCF₃, —OCH₃, —OCH₂CH₃, —F, —OH, —O(CH₂)₂—OH, —O(CH₂)₂—F, —SCH₃, —SCH₂CH₃, —NH—CH₃, —NH—CH₂CH₃, and —N(CH₃)₂.
      Embodiment 46: A compound according to any one of embodiments 2 to 42, wherein
   G is H.
      Embodiment 47: A compound according to any one of embodiments 2 to 42, wherein
   G is C₁₋₈ alkyl optionally substituted one or more times with halogen.
      Embodiment 48: A compound according to any one of embodiments 2 to 42, wherein
   G is C₃₋₁₀ cycloalkyl optionally substituted one or more times with halogen.
      Embodiment 49: A compound according to any one of embodiments 2 to 42, wherein
   G is heterocyclyl optionally substituted one or more times with halogen.
      Embodiment 50: A compound according to any one of embodiments 2 to 42, wherein
   G is —C₁₋₆alkylene-C₃₋₁₀ cycloalkyl optionally substituted one or more times with halogen.
      Embodiment 51: A compound according to any one of embodiments 2 to 42, wherein
   G is NR^{h}R^{k}.
      Embodiment 52: A compound according to any one of embodiments 2 to 42, wherein
   G is —CH₂₋₁₁c.
      Embodiment 53: A compound according to any one of embodiments 2 to 42, wherein
   G is —CH₂CH₂—R^{c}.
      Embodiment 54: A compound according to any one of embodiments 2 to 42, wherein
   G is —CH₂CH₂CH₂—R^{c}.
      Embodiment 55: A compound according to any one of embodiments 2 to 42, wherein
   G is —CH(CH₃)CH₂R^{c}.
      Embodiment 56: A compound according to any one of embodiments 2 to 42, wherein
   G is —CH₂CH(R^{c})CH₃.
      Embodiment 57: A compound according to any one of embodiments 2 to 42, wherein
   G is —CH(R^{c})CH₃.
      Embodiment 58: A compound according to any one of embodiments 2 to 42, wherein
   G is —CH₂C(R^{c})(CH₃)₂.
      Embodiment 59: A compound according to any one of embodiments 2 to 42, wherein
   G is —C(R^{c})(CH₃)₂.
      Embodiment 60: A compound according to any one of embodiments 2 to 42, wherein
   G is imidazol-2-yl, thiazol-2yl, oxazol-2-yl, pyrazol1-yl, furan-2yl, thiophen-2-yl, pyrrol-1-yl, 1H-1,2,4-triazolyl-3-yl, 5-methyl-1H-1,2,4-triazolyl-3-yl, —(CH₂)₁₋₃-(imidazol-2-yl), —(CH₂)₁₋₃-(thiazol-2yl), —(CH₂)₁₋₃-(oxazol-2-yl), —(CH₂)₁₋₃-(pyrazol1-yl), —(CH₂)₁₋₃-(furan-2yl), —(CH₂)₁₋₃-(thiophen-2-yl), —(CH₂)₁₋₃-(pyrrol-1-yl), —(CH₂)₁₋₃-(1H-1,2,4-triazolyl-3-yl), or —(CH₂)₁₋₃-(5-methyl-1H-1,2,4-triazolyl-3-yl).
      Embodiment 61: A compound according to any one of embodiments 2 to 60, wherein
   the compound is in its free (non-salted) form.
      Embodiment 62: A compound according to any one of embodiments 2 to 60, wherein
   the compound is in the form of a pharmaceutically acceptable salt.
      Embodiment 63: A compound according to any one of embodiments 1 to 62, wherein
   any “heterocyclyl” group present in the compound is selected from the group consisting of: azetidin-1-yl, azetidin-2-yl, azetidin-3-yl, pyrrolidin-1-yl, pyrrolidin-2-yl, pyrrolidin-3-yl, tetrahydrofuran-2-yl, tetrahydrofuran-3-yl, tetrahydrothiophen-2-yl, tetrahydrothiophen-3-yl, pyrazolidin-1-yl, pyrazolidin-3-yl, pyrazolidin-4-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,3-dioxolan-2-yl, 1,3-dioxolan-4-yl, 1,3-oxathiolan-2-yl, 1,3-oxathiolane-4-yl, 1,3-oxathiolan-5-yl, 1,2-dithiolan-3-yl, 1,2-dithiolan-4-yl, 1,3-dithiolan-2-yl, 1,3-dithiolan-4-yl, piperidin-1-yl, piperidin-2-yl, piperidin-3-yl, piperidin-4-yl, tetrahydropyran-2-yl, tetrahydropyran-3-yl, tetrahydropyran-4-yl, thian-2-yl, thian-3-yl, thian-4-yl, piperazin-1-yl, piperazin-2-yl, morpholin-2-yl, morpholin-3-yl, morpholin-4-yl, thiomorpholin-2-yl, thiomorpholin-3-yl, thiomorpholin-4-yl, 1,4-dioxan-2-yl, 1,3-dioxan-2-yl, 1,3-dioxan-4-yl, 1,3-dioxan-5-yl, 1,4-dithian-2-yl, 1,3-dithian-2-yl, 1,3-dithian-4-yl, 1,3-dithian-5-yl, 1,2-dithian-3-yl, 1,2-dithian-4-yl, azepan-1-yl, azepan-2-yl, azepan-3-yl, and azepan-4-yl, where each of these named rings may optionally be substituted one or more times with substituents independently selected from halogen, —NH₂, cyano, carboxy, C₁₋₄ alkyl, C₃₋₁₀ cycloalkyl, hydroxyl, thiol, —CF₃, —OCF₃, —O—C₁₋₄ alkyl, —NH—C₁₋₄ alkyl, —N(C₁₋₄ alkyl)₂, —S—C₁₋₄ alkyl, —S(O)₂—C₁₋₄ alkyl, —C(O)—C₁₋₄ alkyl, —C(O)O—C₁₋₄ alkyl, —C(O)NH₂, —C(O)NH—C₁₋₄ alkyl, and —C(O)N(C₁₋₄ alkyl)₂, and where any nitrogen atom in any of these named rings may optionally be oxidized when chemically feasible, and where any sulfur atom in any of these named rings may optionally be oxidized once or twice when chemically feasible.
      Embodiment 64: A compound according to any one of embodiments 1 to 63, wherein any “heteroaryl” group present in the compound is selected from the group consisting of: 1H-pyrrol-1-yl, 1H-pyrrol-2-yl, 1H-pyrrol-3-yl, furan-2-yl, furan-3-yl, thiophen-2-yl, thiophen-3-yl, 1H-imidazol-1-yl, 1H-imidazol-2-yl, 1H-imidazol-4-yl, 1H-imidazol-5-yl, 1H-pyrazol-1-yl, 1H-pyrazol-3-yl, 1H-pyrazol-4-yl, 1H-pyrazol-5-yl, oxazol-2-yl, oxazol-4-yl, oxazol-5-yl, thiazol-2-yl, thiazol-4-yl, thiazol-5-yl, isoxazol-3-yl, isoxazol-4-yl, isoxazol-5-yl, isothiazol-3-yl, isothiazol-4-yl, isothiazol-5-yl, 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl, 1H-1,2,4-triazol-5-yl, furazan-3-yl, pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl, 1,3,5-triazin-2-yl, 1H-indol-1-yl, 1H-indol-2-yl, 1H-indol-3-yl, 2H-isoindol-1-yl, 2H-isoindol-2-yl, quinolin-2-yl, quinolin-3-yl, quinolin-4-yl, isoquinolin-1-yl, isoquinolin-3-yl, isoquinolin-4-yl, benzoxazol-2-yl, benzothiazol-2-yl, 1H-benzimidazol-1-yl, 1H-benzimidazol-2-yl, benzofuran-2-yl, benzofuran-3-yl, benzothiophen-2-yl, and benzothiophen-3-yl, where each of these named rings may optionally be substituted one or more times with substituents independently selected from halogen, —NH₂, cyano, carboxy, C₁₋₄ alkyl, C₃₋₁₀ cycloalkyl, hydroxyl, thiol, —CF₃, —OCF₃, —O—C₁₋₄ alkyl, —NH—C₁₋₄ alkyl, —N(C₁₋₄ alkyl)₂, —S—C₁₋₄ alkyl, —S(O)₂—C₁₋₄ alkyl, —C(O)—C₁₋₄ alkyl, —C(O)O—C₁₋₄ alkyl, —C(O)NH₂, —C(O)NH—C₁₋₄ alkyl, —C(O)N(C₁₋₄ alkyl)₂, and phenyl.
      Embodiment 65: A compound according to any one of embodiments 1 to 64, wherein any “heteroarylene” group present in the compound is selected from the group consisting of: 1H-pyrrol-2,5-diyl, furan-2,5-diyl, thiophen-2,5-diyl, 1H-imidazol-2,4-diyl, 1H-imidazol-2,5-diyl, oxazol-2,4-diyl, oxazol-2,5-diyl, thiazol-2,4-diyl, thiazol-2,5-diyl, 1H-1,2,4-triazol-3,5-diyl, and 2H-isoindol-1,3-diyl, where each of these named rings may optionally be substituted one or more times with substituents independently selected from halogen, —NH₂, cyano, carboxy, —C₁₋₄ alkyl, —C₃₋₁₀ cycloalkyl, hydroxyl, thiol, —CF₃, —OCF₃, —O—C₁₋₄ alkyl, —NH—C₁₋₄ alkyl, —N(C₁₋₄ alkyl)₂, —S—C₁₋₄ alkyl, —S(O)₂—C₁₋₄ alkyl, —C(O)—C₁₋₄ alkyl, —C(O)O—C₁₋₄ alkyl, —C(O)NH₂, —C(O)NH—C₁₋₄ alkyl, —C(O)N(C₁₋₄ alkyl)₂, and phenyl.
      Embodiment 66: A compound according to embodiment 1.
      Embodiment 67: A compound according to embodiment 66, wherein
   R³ is hydrogen.
      Embodiment 68: A compound according to embodiment 66, wherein
   R³ is methyl.
      Embodiment 69: A compound according to embodiment 66, wherein
   R³ is ethyl.
      Embodiment 70: A compound according to embodiment 66, wherein
   R³ is isopropyl.
      Embodiment 71: A compound according to any one of embodiment 66 to 70, wherein
   X¹ is ═N—.
      Embodiment 72: A compound according to any one of embodiments 66 to 70,
   wherein X¹ is ═CH—.
      Embodiment 73: A compound according to any one of embodiments 66 to 72,
   wherein v is 0, 1 or 2.
      Embodiment 74: A compound according to any one of embodiments 66 to 72,
   wherein v is 1 or 2.
      Embodiment 75: A compound according to any one of embodiments 66 to 72,
   wherein v is 1.
      Embodiment 76: A compound according to any one of embodiments 66 to 72,
   wherein v is 1, and R² is attached at either the 5-position or the 6-position of the benzothiazole ring.
      Embodiment 77: A compound according to any one of embodiments 66 to 72,
   wherein v is 1, and R² is attached at the 6-position of the benzothiazole ring.
      Embodiment 78: A compound according to any one of embodiments 66 to 72,
   wherein v is 2, and one R² is attached at the 6-position of the benzothiazole ring.
      Embodiment 79: A compound according to any one of embodiments 66 to 72,
   wherein v is 2, and R² is attached at the 5-position and the 6-position of the benzothiazole ring.
      Embodiment 80: A compound according to any one of embodiments 66 to 79,
   wherein R² is -halogen, —C₁₋₆ alkyl, —CF₃, —OCF₃, —O—R^{f}, or —S(O)_{w}—R^{f}, where the alkyl group is optionally substituted one or more times with substituents independently selected from R^{z}.
      Embodiment 81: A compound according to any one of embodiments 66 to 79,
   wherein R² is -halogen, -methyl, ethyl, isopropyl, —OCH₃, —OCH₂CH₃, —OCH(CH₃)₂, —CF₃, —OCF₃, —SCF₃, —S(O)₂—CH₃, —O-phenyl, —O-(2-pyridyl), —O-(3-pyridyl), or —O-(4-pyridyl).
      Embodiment 82: A compound according to any one of embodiments 66 to 79,
   wherein R² is -halogen, -methyl, ethyl, isopropyl, —OCH₃, —OCH₂CH₃, —OCH(CH₃)₂, —CF₃, —OCF₃, —SCF₃, —S(O)₂—CH₃, or —O-(3-pyridyl).
      Embodiment 83: A compound according to any one of embodiments 66 to 79,
   wherein R² is —Cl, —F, —CF₃, or —OCF₃.
      Embodiment 84: A compound according to any one of embodiments 66 to 79,
   wherein R² is —OCF₃.
      Embodiment 85: A compound according to any one of embodiments 66 to 79,
   wherein R² is —CF₃.
      Embodiment 86: A compound according to any one of embodiments 66 to 79,
   wherein R² is —F.
      Embodiment 87: A compound according to any one of embodiments 66 to 79,
   wherein R² is —Cl.
      Embodiment 88: A compound according to any one of embodiments 66 to 79,
   wherein R² is —SO₂CH₃.
      Embodiment 89: A compound according to any one of embodiments 66 to 79,
   wherein R² is methyl, ethyl, or isopropyl.
      Embodiment 90: A compound according to any one of embodiments 66 to 79,
   wherein R² is methyl.
      Embodiment 91: A compound according to any one of embodiments 66 to 79,
   wherein R² is —OCH₂CH₃.
      Embodiment 92: A compound according to any one of embodiments 66 to 79,
   wherein R² is —O-phenyl.
      Embodiment 93: A compound according to any one of embodiments 66 to 79,
   wherein R² is —O-(2-pyridyl), —O-(3-pyridyl), or —O-(4-pyridyl).
      Embodiment 94: A compound according to any one of embodiments 66 to 79,
   wherein R² is —O-(3-pyridyl).
      Embodiment 95: A compound according to any one of embodiments 66 to 94,
   wherein
   R⁴ is -methyl, -ethyl, -n-propyl, -isopropyl, -n-butyl, -sec-butyl, -isobutyl, -tert-butyl, —(CH₂)₁₋₂—OCH₃, —(CH₂)₁₋₂—F, —(CH₂)₁₋₂—C₁, —(CH₂)₁₋₂—OCF₃, —(CH₂)₁₋₂—NH₂, —(CH₂)₁₋₂—CN, —(CH₂)₁₋₂—OH, —(CH₂)₁₋₂—CF₃, —(CH₂)₁₋₂—CO₂H, —(CH₂)₁₋₂—SH, —(CH₂)₁₋₂—SCH₃, —(CH₂)₁₋₂—S(O)₂CH₃, —(CH₂)₁₋₂—OCH₂CH₃, —(CH₂)₁₋₂—SCH₂CH₃, —(CH₂)₁₋₂—S(O)₂CH₂CH₃, —(CH₂)₁₋₂—NH—CH₃, or —(CH₂)₁₋₂—N(CH₃)₂.
      Embodiment 96: A compound according to any one of embodiments 66 to 94, wherein
   R⁴ is -methyl, -ethyl, -isopropyl, -isobutyl, —CH₂CH₂—OCR₃, —CH₂CH₂—F, CH₂CH₂—NH₂, or —CH₂CH₂—NH—CH₃.
      Embodiment 97: A compound according to any one of embodiments 66 to 94, wherein
   R⁴ is -methyl, -ethyl, -isopropyl, or -isobutyl.
      Embodiment 98: A compound according to any one of embodiments 66 to 94,
   wherein R⁴ is methyl.
      Embodiment 99: A compound according to any one of embodiments 66 to 94,
   wherein R⁴ is -ethyl.
      Embodiment 100: A compound according to any one of embodiments 66 to 94,
   wherein R⁴ is -isopropyl.
      Embodiment 101: A compound according to any one of embodiments 66 to 94,
   wherein R⁴ is -isobutyl.
      Embodiment 102: A compound according to any one of embodiments 66 to 94,
   wherein R⁴ is —CH₂CH₂—OCR₃.
      Embodiment 103: A compound according to any one of embodiments 66 to 94,
   wherein R⁴ is —CH₂CH₂—F.
      Embodiment 104: A compound according to any one of embodiments 66 to 94,
   wherein R⁴ is —CH₂CH₂—NH₂.
      Embodiment 105: A compound according to any one of embodiments 66 to 94,
   wherein R⁴ is —CH₂CH₂—NH—CH₃.
      Embodiment 106: A compound according to any one of embodiments 66 to 105,
   wherein
   R¹ is hydrogen, —OCH₃, —F, —Cl, —NH₂, -cyano, —OH, —CF₃, —OCF₃, —SH, —S—C₁₋₆ alkyl, —S(O)₂—C₁₋₆ alkyl, —CO₂H, —NH—C₁₋₆ alkyl, —N(C₁₋₆ alkyl)₂, or —NH—C₁₋₆ alkyl.
      Embodiment 107: A compound according to any one of embodiments 66 to 105,
   wherein
   R¹ is —OCH₃, —F, —CF₃, —OCF₃, —N(CH₃)₂, —N(CH₂CH₃)₂, or —N(CH₃)(CH₂CH₃).
      Embodiment 108: A compound according to any one of embodiments 66 to 105,
   wherein R¹ is hydrogen, —OCH₃, or —F.
      Embodiment 109: A compound according to any one of embodiments 66 to 105,
   wherein R¹ is hydrogen.
      Embodiment 110: A compound according to any one of embodiments 66 to 105,
   wherein R¹ is —F.
      Embodiment 111: A compound according to any one of embodiments 66 to 105,
   wherein R¹ is —OCH₃.
      Embodiment 112: A compound according to any one of embodiments 66 to 105
   wherein R¹ is —N(CH₂CH₃)₂.
      Embodiment 113: A compound according to any one of embodiments 66 to 112,
   wherein
G is hydrogen, —C₁₋₈ alkyl, —C₃₋₁₀ cycloalkyl, —C₁₋₆ alkylene-C₃₋₁₀ cycloaklyl, heterocyclyl, —C₁₋₆ alkylene-C₃₋₁₀ heterocyclyl, or NR^{h}R^{k}, where the alkyl, alkylene, cycloalkyl, and heterocyclyl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

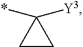

where Y³ is cyclopropyl, —CF₃, —OCF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, or heterocyclylene optionally substituted one or more times with substituents independently selected from R^{x}; or the group -L-G is -cyano;
R¹ is hydrogen or R^{e};
R^{a} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{h},
   i) —S(O)_{w}—R^{h},
   j) —S(O)₂O—R^{h},
   k) —NR^{h}R^{k},
   l) —C(O)—R^{h},
   m) —C(O)—O—R^{h},
   n) —OC(O)—R^{h},
   o) —C(O)NR^{h}R^{k},
   p) —C(O)-heterocyclyl,
   a) —NR^{h}C(O)R^{k},
   r) —OC(O)NR^{h}R^{k},
   s) —NR^{h}C(O)OR^{k},
   t) —NR^{h}C(O)NR^{h}R^{k},
   u) —NR^{h}S(O)_{w}R^{k}, or
   v) —O—(C₁₋₄ alkylene)-O—(C₁₋₄ alkylene)-N(R^{h})C(O)—OR^{k},
where the alkylene, alkyl, cycloalkyl, and heterocyclyl groups are optionally substituted one or more times with substituents independently selected from Rₓ;
R^{h} and R^{k} independently are hydrogen, C₁₋₆ alkyl, C₃₋₁₀ cycloalkyl, or heterocyclyl, where the alkyl, cycloalkyl, and heterocyclyl groups are optionally substituted one or more times with substituents independently selected from R^{x}; or, if R^{h} and R^{k} are both attached to the same nitrogen atom, together with that nitrogen atom may optionally form a heterocyclic ring selected from azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x}; and
R^{x} is R^{y}.
   Embodiment 114: A compound according to any one of embodiments 66 to 112,
   wherein -L-G is not -cyano.
      Embodiment 115: A compound according to any one of embodiments 66 to 112,
   wherein L is —C(O)N(R⁶)—.
      Embodiment 116: A compound according to embodiment 115 wherein
   R⁶ is hydrogen.
      Embodiment 117: A compound according to embodiment 115 wherein
   R⁶ is methyl.
      Embodiment 118: A compound according to embodiment 117 wherein
   G is —N(CH₃)₂.
      Embodiment 119: A compound according to any one of embodiments 66 to 112,
   wherein -L-G is —C(O)NR^{h}R^{k}.
      Embodiment 120: A compound according to embodiment 119, wherein
   NR^{h}R^{k} is pyrrolidino, piperidino, piperazino, 4-methyl-piperazino, or morpholino, where each of the foregoing is optionally substituted once with —(CH₂)₁₋₃—OH.
      Embodiment 121: A compound according to embodiment 120, wherein
   NR^{h}R^{k} is pyrrolidino, 4-(2-hydroxyethyl)-piperazino, or 4-(3-hydroxypropyl)-piperidino.
      Embodiment 122: A compound according to embodiment 119, wherein
   NR^{h}R^{k} is N[(CH₂)₂—OH]2.
      Embodiment 123: A compound according to any one of embodiments 66 to 114,
   wherein L is not —CH₂—C(O)N(R⁶)—.
      Embodiment 124: A compound according to any one of embodiments 66 to 123,
   wherein L is not heterocyclylene.
      Embodiment 125: A compound according to any one of embodiments 66 to 112,
   wherein L is —S(O)₂—.
      Embodiment 126: A compound according to embodiment 125, wherein
   G is methyl or —CF₃.
      Embodiment 127: A compound according to any one of embodiments 66 to 112,
   wherein
   L is heteroarylene optionally substituted one or more times with substituents independently selected from R^{x}.
      Embodiment 128: A compound according to embodiment 127, wherein
   -L-G is imidazol-2-yl, 1,2,4-triazol-3-yl, or 5-methyl-1,2,4-triazol-3-yl.
      Embodiment 129: A compound according to any one of embodiments 66 to 112,
   wherein L is —C(O)—O—.
      Embodiment 130: A compound according to embodiment 129, wherein
   G is hydrogen, or —C₁₋₆ alkyl, where the alkyl group is optionally substituted one or more times with substituents independently selected from R^{c}.
      Embodiment 131: A compound according to embodiment 130, wherein
   G is methyl or ethyl.
      Embodiment 132: A compound according to embodiment 130, wherein
   G is hydrogen.
      Embodiment 133: A compound according to any one of embodiments 66 to 116,
   wherein
   G is —C₁₋₈ alkyl, —C₃₋₁₀ cycloalkyl, —C₁₋₆ alkylene-C₃₋₁₀ cycloaklyl, heterocyclyl, or —C₁₋₆ alkylene-C₃₋₁₀ heterocyclyl, where the alkyl, alkylene, cycloalkyl, and heterocyclyl groups are optionally substituted one or more times with substituents independently selected from R^{c}.
      Embodiment 134: A compound according to embodiment 133, wherein
   G is —C₁₋₈ alkyl optionally substituted one or more times with substituents independently selected from R^{c}.
      Embodiment 135: A compound according to embodiment 134, wherein
   G is methyl, ethyl, isopropyl, n-propyl, n-butyl, sec-butyl, or isobutyl.
      Embodiment 136: A compound according to embodiment 134, wherein
   G is methyl, ethyl, or n-propyl.
      Embodiment 137: A compound according to embodiment 134, wherein
   G is 2-fluoroethyl, 2,2-difluoroethyl, or 2,2,2-trifluoroethyl.
      Embodiment 138: A compound according to embodiment 134, wherein
   G is 2-cyanoethyl.
      Embodiment 139: A compound according to embodiment 134, wherein
   G is —C₁₋₈ alkyl substituted once by —C(O)—O—R^{h}.
      Embodiment 140: A compound according to embodiment 139, wherein
   G is —CH₂—C(O)—O—R^{h}.
      Embodiment 141: A compound according to embodiment 140, wherein
   R^{h} is hydrogen or methyl.
      Embodiment 142: A compound according to embodiment 139, wherein
   G is —CH₂CH₂—C(O)—O—R^{h}.
      Embodiment 143: A compound according to embodiment 142, wherein
   R^{h} is hydrogen or methyl.
      Embodiment 144: A compound according to embodiment 139, wherein
   G is —C(CH₃)₂—C(O)—O—R^{h}.
      Embodiment 145: A compound according to embodiment 144, wherein
   R^{h} is hydrogen or methyl.
      Embodiment 146: A compound according to embodiment 139, wherein
   G is —CH(CH₃)—C(O)—O—R^{h}.
      Embodiment 147: A compound according to embodiment 146, wherein
   R^{h} is hydrogen or methyl.
      Embodiment 148: A compound according to embodiment 134, wherein
   G is —C₁₋₈ alkyl substituted once by —C(O)NR^{h}R^{k}.
      Embodiment 149: A compound according to embodiment 148, wherein
   G is CH₂—C(O)—NR^{h}R^{k}.
      Embodiment 150: A compound according to embodiment 149, wherein
   NR^{h}R^{k} is methylamino, dimethylamino, or diethylamino.
      Embodiment 151: A compound according to embodiment 149, wherein
   NR^{h}R^{k} is thiomorpholino or 1,1-dioxothiomorpholino.
      Embodiment 152: A compound according to embodiment 149, wherein
   NR^{h}R^{k} is morpholino, pyrrolidino, piperidino, piperazino, or 4-methylpiperazino.
      Embodiment 153: A compound according to embodiment 149, wherein
   NR^{h}R^{k} is pyrrolidino, 3-hydroxy-pyrrolidino, 3-methoxy-pyrrolidino, 3-amino-pyrrolidino, 3-(methylamino)-pyrrolidino, 3-(dimethylamino)-pyrrolidino, 2-(hydroxymethyl)-pyrrolidino, 2-(dimethylaminocarbonyl)-pyrrolidino or 3,4-dihydroxy-pyrrolidino.
      Embodiment 154: A compound according to embodiment 149, wherein
   NR^{h}R^{k} is piperazino, 4-methylpiperazino, 4-(methylsulfonyl)-piperazino, or 4-(dimethylaminosulfonyl)-piperazino.
      Embodiment 155: A compound according to embodiment 149, wherein
   NR^{h}R^{k} is piperidino, 3-hydroxypiperidino, 4-hydroxypiperidino, 2-(hydroxymethyl)-piperidino, 3-(hydroxymethyl)-piperidino, 4-(hydroxymethyl)-piperidino, 3-methoxy-piperidino, 4-(methoxymethyl)-piperidino, 4-(fluoromethyl)-piperidino, 4-(trifluoromethyl)-piperidino, 4-cyano-piperidino, 4-carbamoyl-piperidino, 4-(methylamino)-piperidino, 4-(dimethylamino)-piperidino, 4-(methylaminomethyl)-piperidino, or 4-(dimethylaminomethyl)-piperidino.
      Embodiment 156: A compound according to embodiment 149, wherein
   NR^{h}R^{k} is NHR^{k}, where R^{k} is 2-hydroxypropyl, 2-(methylsulfonyl)-ethyl, tetrahydrofuran-3-yl, tetrahydropyran-4-yl, 1-methylpiperidin-4-yl, piperidin-3-yl, or 1-methylpiperidin-3-yl.
      Embodiment 157: A compound according to embodiment 149, wherein
   NR^{h}R^{k} is N(CH₃)R^{k}, where R^{k} is 2-hydroxyethyl, tetrahydropyran-4-yl, pyrrolidin-3-yl, 1-methylpyrrolidin-3-yl, or piperazin-3-yl.
      Embodiment 158: A compound according to embodiment 149, wherein
   NR^{h}R^{k} is N(CH₂CH₂OH)₂.
      Embodiment 159: A compound according to embodiment 148, wherein
   G is —(CH₂)₂₋₃—C(O)—N(CH₃)₂.
      Embodiment 160: A compound according to embodiment 148, wherein
   G is —(CH₂)₃—C(O)-(4-methylpiperazino).
      Embodiment 161: A compound according to embodiment 148, wherein
   G is —CH(CH₃)—C(O)—NR^{h}R^{k}, where NR^{h}R^{k} is methylamino, dimethylamino, 4-methylpiperazino, or morpholino.
      Embodiment 162: A compound according to embodiment 148, wherein
   G is —C(CH₃)₂—C(O)—N(CH₃)₂.
      Embodiment 163: A compound according to embodiment 134, wherein
   G is —CH—[C(O)—N(CH₃)₂]—[CH₂OH], —CH—[C(O)—N(CH₃)₂]—[(CH₂)₄—NH₂], or —CH—[C(O)—N(CH₃)₂]—[(CH₂)₄—N(CH₃)₂].
      Embodiment 164: A compound according to embodiment 134, wherein
   G is —C₁₋₈ alkyl substituted once by —O—R^{h}.
      Embodiment 165: A compound according to embodiment 164, wherein
   G is —(CH₂)₂—O—R^{h}.
      Embodiment 166: A compound according to embodiment 165, wherein
   R^{h} is hydrogen, methyl, or ethyl.
      Embodiment 167: A compound according to embodiment 165, wherein
   R^{h} is trifluoromethyl, 2-fluoroethyl, 3-fluoropropyl, or 2,2-difluoroethyl.
      Embodiment 168: A compound according to embodiment 165, wherein
   R^{h} is tetrahydrofuran-2-ylmethyl.
      Embodiment 169: A compound according to embodiment 165, wherein
   R^{h} is 2-hydroxyethyl.
      Embodiment 170: A compound according to embodiment 165, wherein
   R^{h} is 3-hydroxypropyl.
      Embodiment 171: A compound according to embodiment 165, wherein
   R^{h} is 2-methoxyethyl.
      Embodiment 172: A compound according to embodiment 165, wherein
   R^{h} is 2-(2-hydroxyethoxy)-ethyl.
      Embodiment 173: A compound according to embodiment 165, wherein
   R^{h} is 2-hydroxypropyl or 1-hydroxyprop-2-yl.
      Embodiment 174: A compound according to embodiment 165, wherein
   R^{h} is 2-cyanoethyl, 2-(methylcarbonylamino)-ethyl, or 2-(methylsulfonylamino)-ethyl.
      Embodiment 175: A compound according to embodiment 165, wherein
   R^{h} is 2-aminoethyl, 2-(methylamino)-ethyl, or 2-(dimethylamino)-ethyl.
      Embodiment 176: A compound according to embodiment 165, wherein
   R^{h} is carbamoylmethyl.
      Embodiment 177: A compound according to embodiment 164, wherein
   G is —(CH₂)₃—O—R^{h}.
      Embodiment 178: A compound according to embodiment 177, wherein
   R^{h} is hydrogen, methyl, or ethyl.
      Embodiment 179: A compound according to embodiment 177, wherein
   R^{h} is 2-hydroxyethyl.
      Embodiment 180: A compound according to embodiment 164, wherein
   G is —(CH₂)₄—OH, —(CH₂)₅—OH, —CH₂C(CH₃)₂—OH, —CH₂C(CH₃)₂—OCH₃, —CH₂C(CH₃)₂—CH₂—OH, —CH(CH₃)—CH₂—OCH₃, —(CH₂)₃C(CH₃)₂—CH₂—OH, —(CH₂)₂CH(CH₃)—CH₂—OH, or —(CH₂)₂CH(CH₃)—OH.
      Embodiment 181: A compound according to embodiment 164, wherein
   G is —CH₂CH(CH₃)—O—R^{h}.
      Embodiment 182: A compound according to embodiment 181, wherein
   R^{h} is hydrogen, methyl, or ethyl.
      Embodiment 183: A compound according to embodiment 134, wherein
   G is —CH₂—CH(OH)—CH₂—OH.
      Embodiment 184: A compound according to embodiment 134, wherein
      G is —C₁₋₈ alkyl substituted once by —NR^{h}R^{k}.
      Embodiment 185: A compound according to embodiment 184, wherein
   G is —(CH₂)₂—NR^{h}R^{k}.
      Embodiment 186: A compound according to embodiment 185, wherein
   NR^{h}R^{k} is amino, methylamino, or dimethylamino.
      Embodiment 187: A compound according to embodiment 185, wherein
   NR^{h}R^{k} is methylcarbonylamino.
      Embodiment 188: A compound according to embodiment 185, wherein
   NR^{h}R^{k} is (dimethylamino)methylcarbonylamino, hydroxymethylcarbonylamino, or 1-hydroxyethylcarbonylamino.
      Embodiment 189: A compound according to embodiment 185, wherein
   NR^{h}R^{k} is methylsulfonylamino.
      Embodiment 190: A compound according to embodiment 185, wherein
   NR^{h}R^{k} is piperidino, 4-hydroxypiperidino, or 3-hydroxypiperidino.
      Embodiment 191: A compound according to embodiment 185, wherein
   NR^{h}R^{k} is piperidino, 4,4-difluoropiperidino, or 3,3-difluoropiperidino.
      Embodiment 192: A compound according to embodiment 185, wherein
   NR^{h}R^{k} is 2-oxo-pyrrolidino, 2-oxo-imidazolidino, or 3-oxo-piperazino.
      Embodiment 193: A compound according to embodiment 185, wherein
   NR^{h}R^{k} is piperazino, 4-methylpiperazino, morpholino, or 1,1-dioxo-thiomorpholino.
      Embodiment 194: A compound according to embodiment 184, wherein
   G is —(CH₂)₃—NR^{h}R^{k}.
      Embodiment 195: A compound according to embodiment 194, wherein
   NR^{h}R^{k} is amino, dimethylamino, or diethylamino.
      Embodiment 196: A compound according to embodiment 194, wherein
   NR^{h}R^{k} is piperidino, 4-methylpiperazino, or morpholino.
      Embodiment 197: A compound according to embodiment 184, wherein
   G is —(CH₂)₄—NR^{h}R^{k}.
      Embodiment 198: A compound according to embodiment 197, wherein
   NR^{h}R^{k} is amino, dimethylamino, or diethylamino.
      Embodiment 199: A compound according to embodiment 133, wherein
   G is —C₁₋₆ alkylene-heterocyclyl, where the alkylene and heterocyclyl groups are optionally substituted one or more times with substituents independently selected from R^{c}.
      Embodiment 200: A compound according to embodiment 199, wherein
   G is —CH₂-heterocyclyl, where the heterocyclyl group is optionally substituted once with a substituent selected from R^{c}.
      Embodiment 201: A compound according to embodiment 200, wherein
   the heterocyclyl group is tetrahydropyran-4-yl, tetrahydrofuran-2-yl, 1,4-dioxan-2-yl, morpholin-2-yl, tetrahydropyran-2-yl, piperidin-4-yl, 1-(2-hydroxyethyl)-piperidin-4-yl, 1-(dimethylaminomethylcarbonyl)-piperidin-4-yl, piperazin-2-yl, or 1-methyl-piperazin-2-yl.
      Embodiment 202: A compound according to embodiment 133, wherein
   G is C₃₋₁₀ cycloalkyl optionally substituted one or more times with substituents independently selected from R^{c}.
      Embodiment 203: A compound according to embodiment 202, wherein
   G is 4-hydroxy-cyclohexyl, 4-carboxy-cyclohexyl, or 4-(dimethylaminocarbonyl)-cyclohexyl.
      Embodiment 204: A compound according to embodiment 202, wherein
   G is 1-carboxy-cyclopropyl, 1-(ethoxycarbonyl)-cyclopropyl, or 1-(dimethylamino-carbonyl)-cyclopropyl.
      Embodiment 205: A compound according to embodiment 133, wherein
   G is C₁₋₆ alkylene-C₃₋₁₀ cycloalkyl, where the alkylene and cycloalkyl groups are optionally substituted one or more times with substituents independently selected from R^{c}.
      Embodiment 206: A compound according to embodiment 205, wherein
   G is —CH₂-(4-hydroxy-cyclohexyl).
      Embodiment 207: A compound according to embodiment 205, wherein
   G is —(CH₂)₂-(4-hydroxy-cyclohexyl).
      Embodiment 208: A compound according to embodiment 205, wherein
   G is —CH₂-[4-(hydroxymethyl)-cyclohexyl].
      Embodiment 209: A compound according to embodiment 133, wherein
   G is heterocyclyl optionally substituted one or more times with substituents independently selected from R^{c}.
      Embodiment 210: A compound according to embodiment 209, wherein
   G is piperidin-4-yl, 1-methyl-piperidin-4-yl, 1-carboxy-piperidin-4-yl, 1-(methylsulfonyl)-piperidin-4-yl, 1-(2-hydroxyethyl)-piperidin-4-yl, 1-(dimethyl-aminocarbonyl)piperidin-4-yl, or 1-(dimethylaminomethylcarbonyl)-piperidin-4-yl.
      Embodiment 211: A compound according to embodiment 209, wherein
   G is piperidin-3-yl or 1-(dimethylaminomethylcarbonyl)-piperidin-3-yl.
      Embodiment 212: A compound according to embodiment 209, wherein
   G is 1,1-dioxo-tetrahydrothiophen-3-yl.
      Embodiment 213: A compound according to embodiment 209, wherein
   G is pyrrolidin-3-yl, 1-methyl-pyrrolidin-3-yl, 1-(2-hydroxyethyl)-pyrrolidin-3-yl, 1-(2-hydroxypropyl)-pyrrolidin-3-yl, 1-(2-hydroxy-2-methylpropyl)-pyrrolidin-3-yl, 1-(1-hydroxyethylcarbonyl)-pyrrolidin-3-yl, 1-(2-carboxyethyl)-pyrrolidin-3-yl, or 1-(2-methylsulfonylamino-ethyl)-pyrrolidin-3-yl.
      Embodiment 214: A compound according to embodiment 134, wherein
   G is —C₁₋₆ alkyl substituted once by —S—R^{h}.
      Embodiment 215: A compound according to embodiment 214, wherein
   G is —(CH₂)₂—S—R^{h}.
      Embodiment 216: A compound according to embodiment 215, wherein
   R^{h} is methyl or ethyl.
      Embodiment 217: A compound according to embodiment 215, wherein
   R^{h} is 2-hydroxyethyl.
      Embodiment 218: A compound according to embodiment 214, wherein
   G is —(CH₂)₃—S—R^{h}.
      Embodiment 219: A compound according to embodiment 218, wherein
   R^{h} is methyl.
      Embodiment 220: A compound according to embodiment 134, wherein
   G is —C₁₋₈ alkyl substituted once by —SO₂—R^{h}.
      Embodiment 221: A compound according to embodiment 220, wherein
   G is —(CH₂)₂—SO₂—R^{h}.
      Embodiment 222: A compound according to embodiment 221, wherein
   R^{h} is methyl or ethyl.
      Embodiment 223: A compound according to embodiment 221, wherein
   R^{h} is 2-hydroxyethyl.
      Embodiment 224: A compound according to embodiment 220, wherein
   G is —(CH₂)₃—SO₂—R^{h}.
      Embodiment 225: A compound according to embodiment 224, wherein
   R^{h} is methyl.
      Embodiment 226: A compound according to embodiment 133 wherein
   G is —CH(CH₃)—NR^{h}R^{k}, where NR^{h}R^{k} is pyrrolidino, piperidino, 4-methyl-piperazino, morpholino, or dimethylamino.
      Embodiment 227: A compound according to embodiment 133 wherein
   G is 1-(2-hydroxypropyl)-pyrrolidin-3-yl or 1-(1-hydroxyethylcarbonyl)-pyrrolidin-3-yl.
      Embodiment 228: A compound according to embodiment 133 wherein
   G is 1-(dimethylaminomethylcarbonyl)-piperidin-4-yl.
      Embodiment 229: A compound according to embodiment 133 wherein
   G is —(CH₂)₃₋₅—OH.
      Embodiment 230: A compound according to embodiment 133 wherein
   G is 4-hydroxy-cyclohexylmethyl.
      Embodiment 231: A compound according to embodiment 133 wherein
   G is —(CH₂)₂—NHC(O)—CH₂—N(CH₃)₂.
      Embodiment 232: A compound according to embodiment 133 wherein
   G is 4-hydroxy-cyclohexylmethyl.
      Embodiment 233: A compound according to embodiment 133 wherein
   G is —CH₂—C(O)—NR^{h}R^{k}, where NR^{h}R^{k} is 3-hydroxy-pyrrolidino or 3-(dimethyl-amino)-pyrrolidino.
      Embodiment 234: A compound according to embodiment 133 wherein
   G is —CH₂—C(O)—NR^{h}R^{k}, where NR^{h}R^{k} is morpholino.
      Embodiment 235: A compound according to embodiment 133 wherein
   G is —CH₂—C(O)—NR^{h}R^{k}, where NR^{h}R^{k} is 4-hydroxy-piperidino, 4-methoxy-piperidino, 4-(hydroxymethyl)-piperidino, 3-hydroxy-piperidino, 3-methoxy-piperidino, 3-(hydroxymethyl)-piperidino, or 4,4-difluoropiperidino.
      Embodiment 236: A compound according to embodiment 133 wherein
   G is —CH₂—C(O)—NR^{h}R^{k}, where NR^{h}R^{k} is dimethylamino.
      Embodiment 237: A compound according to embodiment 133 wherein
   G is —(CH₂)₂—O—(CH₂)₂—OH.
      Embodiment 238: A compound according to embodiment 133 wherein
   G is —(CH₂)₂—O—(CH₂)₂—OCH₃.
      Embodiment 239: A compound according to embodiment 133 wherein
   G is —CH₂—CH(CH₃)—OH.
      Embodiment 240: A compound according to any one of embodiments 66 to 112,
   wherein
      L is C(O)NH, and G is C₁₋₈ alkyl substituted once by a heteroaryl group, where the heteroaryl group is optionally substituted one or more times with substituents independently selected from R^{x}.
         Embodiment 241: A compound according to embodiment 240, wherein
   G is —CH₂-(2-furyl), —CH₂-(2-thienyl), —CH₂-(2-oxazolyl), or —CH₂-(2-thiazolyl).
      Embodiment 242: A compound according to embodiment 240, wherein
   G is —(CH₂)₂₋₃-(1-pyrrolyl), —(CH₂)₂₋₃-(1-pyrazolyl), or —(CH₂)₂₋₃-(1-imidazolyl).
      Embodiment 243: A compound according to any one of embodiments 66 to 112,
   wherein
      L is C(O)NH, and G is C₁₋₈ alkyl substituted once by a phenyl group, where the phenyl group is optionally substituted one or more times with substituents independently selected from R^{x}.
         Embodiment 244: A compound according to embodiment 243, wherein
   G is -(—CH₂)₁₋₂-(4-hydroxyphenyl) or -(—CH₂)₁₋₂-(4-methoxy-3-hydroxyphenyl).
      Embodiment 245: A compound according to any one of embodiments 66 to 112,
   wherein
      L is C(O)NH, and G is —CH₂—C(O)NH—CH₂-(4-hydroxyphenyl).
         Embodiment 246: A compound according to any one of embodiments 66 to 112,
   wherein
      L is C(O)NH, and G is —CH₂—C(O)-[4-(pyrimidin-2-yloxy)-piperidino].
         Embodiment 247: A compound according to any one of embodiments 1 to 246,
   wherein X² is ═C(R¹)— and X³ is ═C(-L-G)-.
      Embodiment 248: A compound according to any one of embodiments 1 to 247,
   wherein the compound is in the form of a free acid or a free base.
      Embodiment 249: A compound according to any one of embodiments 1 to 247,
   wherein the compound is in the form of a pharmaceutically acceptable salt.
      Embodiment 250: A compound according to embodiment 1, wherein the compound is a compound from Table A or a pharmaceutically acceptable salt thereof.

TABLE A

| No. | Name |
|---|---|
| 1 | 1-Methyl-2-(6-trifluoromethoxy-benzothiazol-2-ylamino)-1H- |
| | benzoimidazole-5-carboxylic acid methyl amide |
| 2 | 1-Methyl-2-(6-trifluoromethoxy-benzothiazol-2-ylamino)-1H- |
| | benzoimidazole-5-carboxylic acid methyl ester |
| 3 | 1-Methyl-2-(6-trifluoromethoxy-benzothiazol-2-ylamino)-1H- |
| | benzoimidazole-5-carboxylic acid |
| 4 | 1-Methyl-2-(6-trifluoromethoxy-benzothiazol-2-ylamino)-1H- |
| | benzoimidazole-5-carboxylic acid (2-ethoxy-ethyl)-amide |
| 5 | 1-Methyl-2-(6-trifluoromethoxy-benzothiazol-2-ylamino)-1H- |
| | benzoimidazole-5-carboxylic acid cyclopropylmethyl-amide |
| 6 | 1-Methyl-2-(6-trifluoromethoxy-benzothiazol-2-ylamino)-1H- |
| | benzoimidazole-5-carboxylic acid ethylamide |
| 7 | [1-Methyl-2-(6-trifluoromethoxy-benzothiazol-2-ylamino)-1H- |
| | benzoimidazol-5-yl]-pyrrolidin-1-yl-methanone |
| 8 | 1-Methyl-2-(6-trifluoromethoxy-benzothiazol-2-ylamino)-1H- |
| | benzoimidazole-5-carboxylic acid (2-methoxy-ethyl)-amide |
| 9 | 1-Methyl-2-(6-trifluoromethoxy-benzothiazol-2-ylamino)-1H- |
| | benzoimidazole-5-carboxylic acid (2-fluoro-ethyl)-amide |
| 10 | 1-Methyl-2-(6-trifluoromethoxy-benzothiazol-2-ylamino)-1H- |
| | benzoimidazole-5-carboxylic acid (2-hydroxy-ethyl)-amide |
| 11 | 1-Methyl-2-(6-trifluoromethoxy-benzothiazol-2-ylamino)-1H- |
| | benzoimidazole-5-carboxylic acid (3-pyrazol-1-yl-propyl)-amide |
| 12 | 1-Methyl-2-(6-trifluoromethoxy-benzothiazol-2-ylamino)-1H- |
| | benzoimidazole-5-carboxylic acid propylamide |
| 13 | 1-Methyl-2-(6-trifluoromethoxy-benzothiazol-2-ylamino)-1H- |
| | benzoimidazole-5-carboxylic acid (3-hydroxy-propyl)-amide |
| 14 | 1-Methyl-2-(6-trifluoromethoxy-benzothiazol-2-ylamino)-1H- |
| | benzoimidazole-5-carboxylic acid (3-ethoxy-propyl)-amide |
| 15 | 1-Methyl-2-(6-trifluoromethoxy-benzothiazol-2-ylamino)-1H- |
| | benzoimidazole-5-carboxylic acid morpholin-4-ylamide |
| 16 | 1-Methyl-2-(6-trifluoromethoxy-benzothiazol-2-ylamino)-1H- |
| | benzoimidazole-5-carboxylic acid (2,2,2-trifluoro-ethyl)-amide |
| 17 | 1-Methyl-2-(6-trifluoromethoxy-benzothiazol-2-ylamino)-1H- |
| | benzoimidazole-5-carboxylic acid (tetrahydro-pyran-4-ylmethyl)-amide |
| 18 | 1-Methyl-2-(6-trifluoromethoxy-benzothiazol-2-ylamino)-1H- |
| | benzoimidazole-5-carboxylic acid (tetrahydro-furan-2-ylmethyl)-amide |
| 19 | 1-Methyl-2-(6-trifluoromethoxy-benzothiazol-2-ylamino)-1H- |
| | benzoimidazole-5-carboxylic acid (3-methoxy-propyl)-amide |
| 20 | 1-Methyl-2-(6-trifluoromethoxy-benzothiazol-2-ylamino)-1H- |
| | benzoimidazole-5-carboxylic acid (2-methoxy-1-methyl-ethyl)-amide |
| 21 | 1-Methyl-2-(6-trifluoromethoxy-benzothiazol-2-ylamino)-1H- |
| | benzoimidazole-5-carboxylic acid (2-hydroxy-propyl)-amide |
| 22 | 1-Methyl-2-(6-trifluoromethoxy-benzothiazol-2-ylamino)-1H- |
| | benzoimidazole-5-carboxylic acid (2-methoxy-2-methyl-propyl)-amide |
| 23 | 1-Methyl-2-(6-trifluoromethyl-benzothiazol-2-ylamino)-1H- |
| | benzoimidazole-5-carboxylic acid methyl ester |
| 24 | 1-Methyl-2-(6-trifluoromethyl-benzothiazol-2-ylamino)-1H- |
| | benzoimidazole-5-carboxylic acid |
| 25 | 1-Methyl-2-(6-trifluoromethyl-benzothiazol-2-ylamino)-1H- |
| | benzoimidazole-5-carboxylic acid (2-methoxy-ethyl)-amide |
| 26 | 1-Methyl-2-(6-trifluoromethyl-benzothiazol-2-ylamino)-1H- |
| | benzoimidazole-5-carboxylic acid ethylamide |
| 27 | 1-Methyl-2-(6-trifluoromethyl-benzothiazol-2-ylamino)-1H- |
| | benzoimidazole-5-carboxylic acid (2-hydroxy-ethyl)-amide |
| 28 | 2-(6-Chloro-benzothiazol-2-ylamino)-1-methyl-1H-benzoimidazole-5- |
| | carboxylic acid methyl ester |
| 29 | 2-(6-Chloro-benzothiazol-2-ylamino)-1-methyl-1H-benzoimidazole-5- |
| | carboxylic acid |
| 30 | 2-(6-Chloro-benzothiazol-2-ylamino)-1-methyl-1H-benzoimidazole-5- |
| | carboxylic acid (2-hydroxy-ethyl)-amide |
| 31 | 2-(6-Chloro-benzothiazol-2-ylamino)-1-methyl-1H-benzoimidazole-5- |
| | carboxylic acid (2-methoxy-ethyl)-amide |
| 32 | 2-(6-Chloro-benzothiazol-2-ylamino)-1-methyl-1H-benzoimidazole-5- |
| | carboxylic acid ethylamide |
| 33 | 2-(5,6-Difluoro-benzothiazol-2-ylamino)-1-methyl-1H-benzoimidazole-5- |
| | carboxylic acid methyl ester |
| 34 | 2-(5,6-Difluoro-benzothiazol-2-ylamino)-1-methyl-1H-benzoimidazole-5- |
| | carboxylic acid |
| 35 | 2-(5,6-Difluoro-benzothiazol-2-ylamino)-1-methyl-1H-benzoimidazole-5- |
| | carboxylic acid ethylamide |
| 36 | 2-(5,6-Difluoro-benzothiazol-2-ylamino)-1-methyl-1H-benzoimidazole-5- |
| | carboxylic acid (2-hydroxy-ethyl)-amide |
| 37 | 2-(5,6-Difluoro-benzothiazol-2-ylamino)-1-methyl-1H-benzoimidazole-5- |
| | carboxylic acid (2-methoxy-ethyl)-amide |
| 38 | 3-Methyl-2-(6-trifluoromethoxy-benzothiazol-2-ylamino)-3H- |
| | benzoimidazole-5-carboxylic acid methylamide |
| 39 | 6-Fluoro-1-methyl-2-(6-trifluoromethoxy-benzothiazol-2-ylamino)-1H- |
| | benzoimidazole-5-carboxylic acid (2-methoxy-ethyl)-amide |
| 40 | 6-Fluoro-1-methyl-2-(6-trifluoromethoxy-benzothiazol-2-ylamino)-1H- |
| | benzoimidazole-5-carboxylic acid methyl ester |
| 41 | 6-Fluoro-1-methyl-2-(6-trifluoromethoxy-benzothiazol-2-ylamino)-1H- |
| | benzoimidazole-5-carboxylic acid |
| 42 | 6-Fluoro-1-methyl-2-(6-trifluoromethoxy-benzothiazol-2-ylamino)-1H- |
| | benzoimidazole-5-carboxylic acid ethylamide |
| 43 | 1-Methyl-2-(6-trifluoromethoxy-benzothiazol-2-ylamino)-1H- |
| | benzoimidazole-5-carboxylic acid (2-morpholin-4-yl-ethyl)-amide |
| 44 | 1-Methyl-2-(6-trifluoromethoxy-benzothiazol-2-ylamino)-1H- |
| | benzoimidazole-5-carboxylic acid (2-trifluoromethoxy-ethyl)-amide |
| 45 | 1-Methyl-2-(6-trifluoromethoxy-benzothiazol-2-ylamino)-1H- |
| | benzoimidazole-5-carboxylic acid (2-hydroxy-2-methyl-propyl)-amide |
| 46 | 1-Methyl-2-(6-trifluoromethoxy-benzothiazol-2-ylamino)-1H- |
| | benzoimidazole-5-carboxylic acid [2-(2-hydroxy-ethoxy)-ethyl]-amide |
| 47 | 1-Methyl-2-(6-trifluoromethoxy-benzothiazol-2-ylamino)-1H- |
| | benzoimidazole-5-carboxylic acid [2-(2-fluoro-ethoxy)-ethyl]-amide |
| 48 | 1-Methyl-2-(6-trifluoromethoxy-benzothiazol-2-ylamino)-1H- |
| | benzoimidazole-5-carboxylic acid (furan-2-ylmethyl)-amide |
| 49 | 1-Methyl-2-(6-trifluoromethoxy-benzothiazol-2-ylamino)-1H- |
| | benzoimidazole-5-carboxylic acid ([1,4]dioxan-2-ylmethyl)-amide |
| 50 | 1-Methyl-2-(6-trifluoromethoxy-benzothiazol-2-ylamino)-1H- |
| | benzoimidazole-5-carboxylic acid ((S)-2-hydroxy-propyl)-amide |
| 51 | 1-Methyl-2-(6-trifluoromethoxy-benzothiazol-2-ylamino)-1H- |
| | benzoimidazole-5-carboxylic acid ((R)-2-hydroxy-propyl)-amide |
| 52 | 1-Methyl-2-(6-trifluoromethoxy-benzothiazol-2-ylamino)-1H- |
| | benzoimidazole-5-carboxylic acid (trans-4-hydroxy-cyclohexyl)-amide |
| 53 | 1-Methyl-2-(6-trifluoromethoxy-benzothiazol-2-ylamino)-1H- |
| | benzoimidazole-5-carboxylic acid [2-(tetrahydro-furan-2-ylmethoxy)- |
| | ethyl]-amide |
| 54 | 1-Methyl-2-(6-trifluoromethoxy-benzothiazol-2-ylamino)-1H- |
| | benzoimidazole-5-carboxylic acid (2-ethoxy-propyl)-amide |
| 55 | 2-({[1-Methyl-2-(6-trifluoromethoxy-benzothiazol-2-ylamino)-1H- |
| | benzoimidazole-5-carbonyl]-amino}-methyl)-morpholine-4-carboxylic acid |
| | tert-butyl ester |
| 56 | 1-Methyl-2-(6-trifluoromethoxy-benzothiazol-2-ylamino)-1H- |
| | benzoimidazole-5-carboxylic acid (morpholin-2-ylmethyl)-amide |
| | hydrochloride |
| 57 | 6-Fluoro-1-methyl-2-(6-trifluoromethoxy-benzothiazol-2-ylamino)-1H- |
| | benzoimidazole-5-carboxylic acid (2-ethoxy-ethyl)-amide |
| 58 | 6-Fluoro-1-methyl-2-(6-trifluoromethoxy-benzothiazol-2-ylamino)-1H- |
| | benzoimidazole-5-carboxylic acid dimethylcarbamoylmethyl-amide |
| 59 | 6-Fluoro-1-methyl-2-(6-trifluoromethoxy-benzothiazol-2-ylamino)-1H- |
| | benzoimidazole-5-carboxylic acid (2-morpholin-4-yl-ethyl)-amide |
| 60 | 6-Fluoro-1-methyl-2-(6-trifluoromethoxy-benzothiazol-2-ylamino)-1H- |
| | benzoimidazole-5-carboxylic acid (2-hydroxy-propyl)-amide |
| 61 | 6-Methoxy-1-methyl-2-(6-trifluoromethoxy-benzothiazol-2-ylamino)-1H- |
| | benzoimidazole-5-carboxylic acid methyl ester |
| 62 | 6-Methoxy-1-methyl-2-(6-trifluoromethoxy-benzothiazol-2-ylamino)-1H- |
| | benzoimidazole-5-carboxylic acid |
| 63 | 6-Methoxy-1-methyl-2-(6-trifluoromethoxy-benzothiazol-2-ylamino)-1H- |
| | benzoimidazole-5-carboxylic acid dimethylcarbamoyl-methyl-amide |
| 64 | 6-Methoxy-1-methyl-2-(6-trifluoromethoxy-benzothiazol-2-ylamino)-1H- |
| | benzoimidazole-5-carboxylic acid ethylamide |
| 65 | 6-Methoxy-1-methyl-2-(6-trifluoromethoxy-benzothiazol-2-ylamino)-1H- |
| | benzoimidazole-5-carboxylic acid (2-ethoxy-ethyl)-amide |
| 66 | 6-Methoxy-1-methyl-2-(6-trifluoromethoxy-benzothiazol-2-ylamino)-1H- |
| | benzoimidazole-5-carboxylic acid (2-morpholin-4-yl-ethyl)-amide |
| 67 | 6-Methoxy-1-methyl-2-(6-trifluoromethoxy-benzothiazol-2-ylamino)-1H- |
| | benzoimidazole-5-carboxylic acid (2-methoxy-ethyl)-amide |
| 68 | 6-Methoxy-1-methyl-2-(6-trifluoromethoxy-benzothiazol-2-ylamino)-1H- |
| | benzoimidazole-5-carboxylic acid (2-hydroxy-propyl)-amide |
| 69 | 6-Diethylamino-1-methyl-2-(6-trifluoromethoxy-benzothiazol-2-ylamino)- |
| | 1H-benzoimidazole-5-carboxylic acid methyl ester |
| 70 | 6-Diethylamino-1-methyl-2-(6-trifluoromethoxy-benzothiazol-2-ylamino)- |
| | 1H-benzoimidazole-5-carboxylic acid |
| 71 | 3-Methyl-2-(6-trifluoromethoxy-benzothiazol-2-ylamino)-3H-imidazo[4,5- |
| | b]pyridine-6-carboxylic acid ethyl ester |
| 72 | 3-Methyl-2-(6-trifluoromethoxy-benzothiazol-2-ylamino)-3H-imidazo[4,5- |
| | b]pyridine-6-carboxylic acid |
| 73 | 3-Methyl-2-(6-trifluoromethoxy-benzothiazol-2-ylamino)-3H-imidazo[4,5- |
| | b]pyridine-6-carboxylic acid (2-methoxy-ethyl)-amide |
| 74 | 3-Methyl-2-(6-trifluoromethoxy-benzothiazol-2-ylamino)-3H-imidazo[4,5- |
| | b]pyridine-6-carboxylic acid dimethylcarbamoylmethyl-amide |
| 75 | 3-Methyl-2-(6-trifluoromethoxy-benzothiazol-2-ylamino)-3H-imidazo[4,5- |
| | b]pyridine-6-carboxylic acid (2-ethoxy-ethyl)-amide |
| 76 | 3-Methyl-2-(6-trifluoromethoxy-benzothiazol-2-ylamino)-3H-imidazo[4,5- |
| | b]pyridine-6-carboxylic acid ethylamide |
| 77 | 3-Methyl-2-(6-trifluoromethoxy-benzothiazol-2-ylamino)-3H-imidazo[4,5- |
| | b]pyridine-6-carboxylic acid (2-morpholin-4-yl-ethyl)-amide |
| 78 | 3-Methyl-2-(6-trifluoromethoxy-benzothiazol-2-ylamino)-3H-imidazo[4,5- |
| | b]pyridine-6-carboxylic acid (2-hydroxy-propyl)-amide |
| 79 | {[1-Methyl-2-(6-trifluoromethoxy-benzothiazol-2-ylamino)-1H- |
| | benzoimidazole-5-carbonyl]-amino}-acetic acid methyl ester |
| 80 | 1-Methyl-2-(6-trifluoromethoxy-benzothiazol-2-ylamino)-1H- |
| | benzoimidazole-5-carboxylic acid dimethylcarbamoylmethyl-amide |
| 81 | 1-Methyl-2-(6-trifluoromethoxy-benzothiazol-2-ylamino)-1H- |
| | benzoimidazole-5-carboxylic acid ((S)-1-ethylcarbamoyl-ethyl)-amide |
| 82 | 1-Methyl-2-(6-trifluoromethoxy-benzothiazol-2-ylamino)-1H- |
| | benzoimidazole-5-carboxylic acid (2-dimethylamino-ethyl)-amide |
| 83 | {[1-Methyl-2-(6-trifluoromethoxy-benzothiazol-2-ylamino)-1H- |
| | benzoimidazole-5-carbonyl]-amino}-acetic acid |
| 84 | 1-Methyl-2-(6-trifluoromethyl-benzothiazol-2-ylamino)-1H- |
| | benzoimidazole-5-carboxylic acid methylamide |
| 85 | 1-Methyl-2-(6-trifluoromethyl-benzothiazol-2-ylamino)-1H- |
| | benzoimidazole-5-carboxylic acid (2-ethoxy-ethyl)-amide |
| 86 | 2-(5,6-Difluoro-benzothiazol-2-ylamino)-1-methyl-1H-benzoimidazole-5- |
| | carboxylic acid methylamide |
| 87 | 2-(5,6-Difluoro-benzothiazol-2-ylamino)-1-methyl-1H-benzoimidazole-5- |
| | carboxylic acid (2-ethoxy-ethyl)-amide |
| 88 | 2-(6-Chloro-benzothiazol-2-ylamino)-1-methyl-1H-benzoimidazole-5- |
| | carboxylic acid methylamide |
| 89 | 2-(6-Chloro-benzothiazol-2-ylamino)-1-methyl-1H-benzoimidazole-5- |
| | carboxylic acid (2-ethoxy-ethyl)-amide |
| 90 | 1-Methyl-2-(6-trifluoromethoxy-benzothiazol-2-ylamino)-1H- |
| | benzoimidazole-5-carboxylic acid (1-methanesulfonyl-piperidin-4-yl)- |
| | amide |
| 91 | {[1-Methyl-2-(6-trifluoromethoxy-benzothiazol-2-ylamino)-1H- |
| | benzoimidazole-5-carbonyl]-amino}-acetic acid tert-butyl ester |
| 92 | 4-{[1-Methyl-2-(6-trifluoromethoxy-benzothiazol-2-ylamino)-1H- |
| | benzoimidazole-5-carbonyl]-amino}-piperidine-1-carboxylic acid tert-butyl |
| | ester |
| 93 | 1-Methyl-2-(6-trifluoromethoxy-benzothiazol-2-ylamino)-1H- |
| | benzoimidazole-5-carboxylic acid piperidin-4-ylamide hydrochloride |
| 94 | 3-{[1-Methyl-2-(6-trifluoromethoxy-benzothiazol-2-ylamino)-1H- |
| | benzoimidazole-5-carbonyl]-amino}-piperidine-1-carboxylic acid tert-butyl |
| | ester |
| 95 | 1-Methyl-2-(6-trifluoromethoxy-benzothiazol-2-ylamino)-1H- |
| | benzoimidazole-5-carboxylic acid piperidin-3-ylamide hydrochloride |
| 96 | 1-Methyl-2-(6-trifluoromethoxy-benzothiazol-2-ylamino)-1H- |
| | benzoimidazole-5-carboxylic acid (thiazol-2-ylmethyl)-amide |
| 97 | 3-{[1-Methyl-2-(6-trifluoromethoxy-benzothiazol-2-ylamino)-1H- |
| | benzoimidazole-5-carbonyl]-amino}-propionic acid methyl ester |
| 98 | 3-{[2-(6-Trifluoromethoxy-benzothiazol-2-ylamino)-1-methyl-1H- |
| | benzimidazole-5-carbonyl]-amino}-propionic acid |
| 99 | 1-Methyl-2-(5-trifluoromethoxy-benzothiazol-2-ylamino)-1H- |
| | benzoimidazole-5-carboxylic acid methyl ester |
| 100 | 1-Methyl-2-(6-trifluoromethoxy-benzothiazol-2-ylamino)-1H- |
| | benzoimidazole-5-carboxylic acid (2-acetylamino-ethyl)-amide |
| 101 | 1-Methyl-2-(6-trifluoromethoxy-benzothiazol-2-ylamino)-1H- |
| | benzoimidazole-5-carboxylic acid (2-methylsulfanyl-ethyl)-amide |
| 102 | 1-Methyl-2-(6-trifluoromethoxy-benzothiazol-2-ylamino)-1H- |
| | benzoimidazole-5-carboxylic acid (2-methanesulfonyl-ethyl)-amide |
| 103 | (2-{[1-Methyl-2-(6-trifluoromethoxy-benzothiazol-2-ylamino)-1H- |
| | benzoimidazole-5-carbonyl]-amino}-ethyl)-carbamic acid tert-butyl ester |
| 104 | 1-Methyl-2-(6-trifluoromethoxy-benzothiazol-2-ylamino)-1H- |
| | benzoimidazole-5-carboxylic acid (2-amino-ethyl)-amide hydrochloride |
| 105 | 1-Methyl-2-(6-trifluoromethoxy-benzothiazol-2-ylamino)-1H- |
| | benzoimidazole-5-carboxylic acid (2-methylamino-ethyl)-amide |
| 106 | 1-Methyl-2-(6-trifluoromethoxy-benzothiazol-2-ylamino)-1H- |
| | benzimidazole-5-carboxylic acid trimethylhydrazide |
| 107 | 1-Methyl-2-(6-trifluoromethoxy-benzothiazol-2-ylamino)-1H- |
| | benzoimidazole-5-carboxylic acid (2-ethylsulfanyl-ethyl)-amide |
| 108 | 1-Methyl-2-(6-trifluoromethoxy-benzothiazol-2-ylamino)-1H- |
| | benzoimidazole-5-carboxylic acid (3-methylsulfanyl-propyl)-amide |
| 109 | 1-Methyl-2-(6-trifluoromethoxy-benzothiazol-2-ylamino)-1H- |
| | benzimidazole-5-carboxylic acid (2-ethanesulfonyl-ethyl)-amide |
| 110 | 1-Methyl-2-(6-trifluoromethoxy-benzothiazol-2-ylamino)-1H- |
| | benzimidazole-5-carboxylic acid (3-methanesulfonyl-propyl)-amide |
| 111 | 2-(5-Fluoro-benzothiazol-2-ylamino)-1-methyl-1H-benzoimidazole-5- |
| | carboxylic acid methyl ester |
| 112 | 2-(6-Fluoro-benzothiazol-2-ylamino)-1-methyl-1H-benzoimidazole-5- |
| | carboxylic acid methyl ester |
| 113 | 2-(6-Methanesulfonyl-benzothiazol-2-ylamino)-1-methyl-1H- |
| | benzoimidazole-5-carboxylic acid methyl ester |
| 114 | 1-Methyl-2-(6-methyl-benzothiazol-2-ylamino)-1H-benzoimidazole-5- |
| | carboxylic acid methyl ester |
| 115 | 1-Methyl-2-(5-trifluoromethoxy-benzothiazol-2-ylamino)-1H- |
| | benzimidazole-5-carboxylic acid methylamide |
| 116 | 1-Methyl-2-(5-trifluoromethoxy-benzothiazol-2-ylamino)-1H- |
| | benzimidazole-5-carboxylic acid (2-methoxy-ethyl)-amide |
| 117 | 2-(5-Fluoro-benzothiazol-2-ylamino)-1-methyl-1H-benzimidazole-5- |
| | carboxylic acid |
| 118 | 2-(6-Fluoro-benzothiazol-2-ylamino)-1-methyl-1H-benzimidazole-5- |
| | carboxylic acid |
| 119 | 2-(6-Methanesulfonyl-benzothiazol-2-ylamino)-1-methyl-1H- |
| | benzimidazole-5-carboxylic acid |
| 120 | 1-Methyl-2-(6-methyl-benzothiazol-2-ylamino)-1H-benzimidazole-5- |
| | carboxylic acid |
| 121 | 1-Methyl-2-(6-trifluoromethoxy-benzothiazol-2-ylamino)-1H- |
| | benzimidazole-5-carboxylic acid (1,1-dioxo-tetrahydro-1λ⁶-thiophen-3-yl)- |
| | amide |
| 122 | 2-(5-Fluoro-benzothiazol-2-ylamino)-1-methyl-1H-benzimidazole-5- |
| | carboxylic acid methylamide |
| 123 | 2-(5-Fluoro-benzothiazol-2-ylamino)-1-methyl-1H-benzimidazole-5- |
| | carboxylic acid (2-methoxy-ethyl)-amide |
| 124 | 2-(6-Fluoro-benzothiazol-2-ylamino)-1-methyl-1H-benzimidazole-5- |
| | carboxylic acid methylamide |
| 125 | 2-(6-Fluoro-benzothiazol-2-ylamino)-1-methyl-1H-benzimidazole-5- |
| | carboxylic acid (2-methoxy-ethyl)-amide |
| 126 | 2-(6-Methanesulfonyl-benzothiazol-2-ylamino)-1-methyl-1H- |
| | benzimidazole-5-carboxylic acid methylamide |
| 127 | 2-(6-Methanesulfonyl-benzothiazol-2-ylamino)-1-methyl-1H- |
| | benzimidazole-5-carboxylic acid (2-methoxy-ethyl)-amide |
| 128 | 2-(6-Methyl-benzothiazol-2-ylamino)-1-methyl-1H-benzimidazole-5- |
| | carboxylic acid methylamide |
| 129 | 2-(6-Methyl-benzothiazol-2-ylamino)-1-methyl-1H-benzimidazole-5- |
| | carboxylic acid (2-methoxy-ethyl)-amide |
| 130 | 2-(6-Methanesulfonyl-benzothiazol-2-ylamino)-1-methyl-1H- |
| | benzimidazole-5-carboxylic acid (2-methylsulfanyl-ethyl)-amide |
| 131 | 2-(6-Methanesulfonyl-benzothiazol-2-ylamino)-1-methyl-1H- |
| | benzimidazole-5-carboxylic acid (2-methylsulfonyl-ethyl)-amide |
| 132 | 1-Methyl-2-(6-trifluoromethylsulfanyl-benzothiazol-2-ylamino)-1H- |
| | benzoimidazole-5-carboxylic acid methyl ester |
| 133 | 2-(6-Chloro-benzothiazol-2-ylamino)-1-methyl-1H-benzimidazole-5- |
| | carboxylic acid dimethylcarbamoylmethyl-amide |
| 134 | 1-Methyl-2-(6-trifluoromethyl-benzothiazol-2-ylamino)-1H-benzimidazole- |
| | 5-carboxylic acid dimethylcarbamoylmethyl-amide |
| 135 | 1-Methyl-2-(6-trifluoromethoxy-benzothiazol-2-ylamino)-1H- |
| | benzimidazole-5-carboxylic acid (2-dimethylcarbamoyl-ethyl)-amide |
| 136 | 3-{[1-Methyl-2-(6-trifluoromethoxy-benzothiazol-2-ylamino)-1H- |
| | benzimidazole-5-carbonyl]-amino}-propionic acid tert-butyl ester |
| 137 | 1-Methyl-2-(6-trifluoromethoxy-benzothiazol-2-ylamino)-1H- |
| | benzimidazole-5-carboxylic acid [2-(4-methyl-piperazin-1-yl)-2-oxo-ethyl]- |
| | amide |
| 138 | 1-Methyl-2-(6-trifluoromethoxy-benzothiazol-2-ylamino)-1H- |
| | benzimidazole-5-carboxylic acid (2-morpholin-4-yl-2-oxo-ethyl)-amide |
| 139 | 1-Methyl-2-(6-trifluoromethoxy-benzothiazol-2-ylamino)-1H- |
| | benzimidazole-5-carboxylic acid methylcarbamoylmethyl-amide |
| 140 | 1-Methyl-2-(6-trifluoromethoxy-benzothiazol-2-ylamino)-1H- |
| | benzimidazole-5-carboxylic acid diethylcarbamoylmethyl-amide |
| 141 | 1-Methyl-2-(6-trifluoromethoxy-benzothiazol-2-ylamino)-1H- |
| | benzimidazole-5-carboxylic acid (2-oxo-2-pyrrolidin-1-yl-ethyl)-amide |
| 142 | 4-(2-{[1-Methyl-2-(6-trifluoromethoxy-benzothiazol-2-ylamino)-1H- |
| | benzimidazole-5-carbonyl]-amino}-acetyl)-piperazine-1-carboxylic acid |
| | tert-butyl ester |
| 143 | (S)-2-{[1-Methyl-2-(6-trifluoromethoxy-benzothiazol-2-ylamino)-1H- |
| | benzoimidazole-5-carbonyl]-amino}-propionic acid methyl ester |
| 144 | 1-{[1-Methyl-2-(6-trifluoromethoxy-benzothiazol-2-ylamino)-1H- |
| | benzimidazole-5-carbonyl]-amino}-cyclopropanecarboxylic acid ethyl ester |
| 145 | 2-Methyl-2-{[1-methyl-2-(6-trifluoromethoxy-benzothiazol-2-ylamino)-1H- |
| | benzimidazole-5-carbonyl]-amino}-propionic acid methyl ester |
| 146 | (S)-2-{[1-Methyl-2-(6-trifluoromethoxy-benzothiazol-2-ylamino)-1H- |
| | benzoimidazole-5-carbonyl]-amino}-propionic acid |
| 147 | 1-{[1-Methyl-2-(6-trifluoromethoxy-benzothiazol-2-ylamino)-1H- |
| | benzimidazole-5-carbonyl]-amino}-cyclopropanecarboxylic acid |
| 148 | 2-Methyl-2-{[1-methyl-2-(6-trifluoromethoxy-benzothiazol-2-ylamino)-1H- |
| | benzimidazole-5-carbonyl]-amino}-propionic acid |
| 149 | 1-Methyl-2-(6-trifluoromethoxy-benzothiazol-2-ylamino)-1H- |
| | benzoimidazole-5-carboxylic acid ((S)-1-dimethylcarbamoyl-ethyl)-amide |
| 150 | 1-Methyl-2-(6-trifluoromethoxy-benzothiazol-2-ylamino)-1H- |
| | benzimidazole-5-carboxylic acid (1-dimethylcarbamoyl-cyclopropyl)-amide |
| 151 | 1-Methyl-2-(6-trifluoromethoxy-benzothiazol-2-ylamino)-1H- |
| | benzimidazole-5-carboxylic acid (1-dimethylcarbamoyl-1-methyl-ethyl)- |
| | amide |
| 152 | 1-Methyl-2-(6-trifluoromethoxy-benzothiazol-2-ylamino)-1H- |
| | benzimidazole-5-carboxylic acid (2-oxo-2-piperazin-1-yl-ethyl)-amide |
| | hydrochloride |
| 153 | 1-Ethyl-2-(6-trifluoromethoxy-benzothiazol-2-ylamino)-1H- |
| | benzoimidazole-5-carboxylic acid methyl ester |
| 154 | 1-Ethyl-2-(6-trifluoromethoxy-benzothiazol-2-ylamino)-1H- |
| | benzoimidazole-5-carboxylic acid |
| 155 | 1-Ethyl-2-(6-trifluoromethoxy-benzothiazol-2-ylamino)-1H- |
| | benzoimidazole-5-carboxylic acid methylamide |
| 156 | 1-Ethyl-2-(6-trifluoromethoxy-benzothiazol-2-ylamino)-1H- |
| | benzoimidazole-5-carboxylic acid ethylamide |
| 157 | 1-Ethyl-2-(6-trifluoromethoxy-benzothiazol-2-ylamino)-1H- |
| | benzoimidazole-5-carboxylic acid (2-ethoxy-ethyl)-amide |
| 158 | 1-Isopropyl-2-(6-trifluoromethoxy-benzothiazol-2-ylamino)-1H |
| | benzoimidazole-5-carboxylic acid methyl ester |
| 159 | 1-Isopropyl-2-(6-trifluoromethoxy-benzothiazol-2-ylamino)-1H- |
| | benzoimidazole-5-carboxylic acid |
| 160 | 1-Isopropyl-2-(6-trifluoromethoxy-benzothiazol-2-ylamino)-1H- |
| | benzoimidazole-5-carboxylic acid methylamide |
| 161 | 1-Isopropyl-2-(6-trifluoromethoxy-benzothiazol-2-ylamino)-1H- |
| | benzoimidazole-5-carboxylic acid ethylamide |
| 162 | 1-Isobutyl-2-(6-trifluoromethoxy-benzothiazol-2-ylamino)-1H- |
| | benzoimidazole-5-carboxylic acid methyl ester |
| 163 | 1-Isobutyl-2-(6-trifluoromethoxy-benzothiazol-2-ylamino)-1H- |
| | benzoimidazole-5-carboxylic acid |
| 164 | 1-Isobutyl-2-(6-trifluoromethoxy-benzothiazol-2-ylamino)-1H- |
| | benzoimidazole-5-carboxylic acid methylamide |
| 165 | 1-Isobutyl-2-(6-trifluoromethoxy-benzothiazol-2-ylamino)-1H- |
| | benzoimidazole-5-carboxylic acid ethylamide |
| 166 | 1-(2-Methoxy-ethyl)-2-(6-trifluoromethoxy-benzothiazol-2-ylamino)-1H- |
| | benzoimidazole-5-carboxylic acid |
| 167 | 1-(2-Methoxy-ethyl)-2-(6-trifluoromethoxy-benzothiazol-2-ylamino)-1H- |
| | benzoimidazole-5-carboxylic acid methylamide |
| 168 | 1-(2-Methoxy-ethyl)-2-(6-trifluoromethoxy-benzothiazol-2-ylamino)-1H- |
| | benzoimidazole-5-carboxylic acid (2-methoxy-ethyl)-amide |
| 169 | 1-(2-Methoxy-ethyl)-2-(6-trifluoromethoxy-benzothiazol-2-ylamino)-1H- |
| | benzoimidazole-5-carboxylic acid (2-ethoxy-ethyl)-amide |
| 170 | 1-(2-Fluoro-ethyl)-2-(6-trifluoromethoxy-benzothiazol-2-ylamino)-1H- |
| | benzoimidazole-5-carboxylic acid methylamide |
| 171 | 1-(2-Fluoro-ethyl)-2-(6-trifluoromethoxy-benzothiazol-2-ylamino)-1H- |
| | benzoimidazole-5-carboxylic acid (2-methoxy-ethyl)-amide |
| 172 | 1-(2-Fluoro-ethyl)-2-(6-trifluoromethoxy-benzothiazol-2-ylamino)-1H- |
| | benzoimidazole-5-carboxylic acid (2-ethoxy-ethyl)-amide |
| 173 | 1-(2-Amino-ethyl)-2-(6-trifluoromethoxy-benzothiazol-2-ylamino)-1H- |
| | benzoimidazole-5-carboxylic acid methylamide hydrochloride |
| 174 | 2-(6-Chloro-benzothiazol-2-ylamino)-1-ethyl-1H-benzoimidazole-5- |
| | carboxylic acid methylamide |
| 175 | 2-(6-Chloro-benzothiazol-2-ylamino)-1-ethyl-1H-benzoimidazole-5- |
| | carboxylic acid ethylamide |
| 176 | 2-(6-Chloro-benzothiazol-2-ylamino)-1-ethyl-1H-benzoimidazole-5- |
| | carboxylic acid (2-fluoro-ethyl)-amide |
| 177 | 2-(6-Chloro-benzothiazol-2-ylamino)-1-ethyl-1H-benzoimidazole-5- |
| | carboxylic acid (2-methoxy-ethyl)-amide |
| 178 | 2-(6-Chloro-benzothiazol-2-ylamino)-1-ethyl-1H-benzoimidazole-5- |
| | carboxylic acid (2-methoxy-2-methyl-propyl)-amide |
| 179 | 2-(6-Chloro-benzothiazol-2-ylamino)-1-ethyl-1H-benzoimidazole-5- |
| | carboxylic acid (2-ethoxy-ethyl)-amide |
| 180 | 1-Ethyl-2-(6-trifluoromethyl-benzothiazol-2-ylamino)-1H-benzoimidazole- |
| | 5-carboxylic acid methylamide |
| 181 | 1-Ethyl-2-(6-trifluoromethyl-benzothiazol-2-ylamino)-1H-benzoimidazole- |
| | 5-carboxylic acid ethylamide |
| 182 | 1-Ethyl-2-(6-trifluoromethyl-benzothiazol-2-ylamino)-1H-benzoimidazole- |
| | 5-carboxylic acid (2-methoxy-ethyl)-amide |
| 183 | 1-Ethyl-2-(6-trifluoromethyl-benzothiazol-2-ylamino)-1H-benzoimidazole- |
| | 5-carboxylic acid (2-ethoxy-ethyl)-amide |
| 184 | 1-Ethyl-2-(6-trifluoromethyl-benzothiazol-2-ylamino)-1H-benzoimidazole- |
| | 5-carboxylic acid (2-methoxy-2-methyl-propyl)-amide |
| 185 | 1-Ethyl-2-(6-trifluoromethyl-benzothiazol-2-ylamino)-1H-benzoimidazole- |
| | 5-carboxylic acid (2-methylsulfanyl-ethyl)-amide |
| 186 | 1-Ethyl-2-(6-trifluoromethoxy-benzothiazol-2-ylamino)-1H- |
| | benzoimidazole-5-carboxylic acid dimethylcarbamoylmethyl-amide |
| 187 | 1-Ethyl-2-(6-trifluoromethyl-benzothiazol-2-ylamino)-1H-benzoimidazole- |
| | 5-carboxylic acid dimethylcarbamoylmethyl-amide |
| 188 | 1-(2-Methoxy-ethyl)-2-(6-trifluoromethoxy-benzothiazol-2-ylamino)-1H- |
| | benzoimidazole-5-carboxylic acid dimethylcarbamoylmethyl-amide |
| 189 | 1-(2-Methoxy-ethyl)-2-(6-trifluoromethoxy-benzothiazol-2-ylamino)-1H- |
| | benzoimidazole-5-carboxylic acid [2-(4-methyl-piperazin-1-yl)-2-oxo-ethyl]- |
| | amide |
| 190 | 1-Ethyl-2-(6-trifluoromethoxy-benzothiazol-2-ylamino)-1H- |
| | benzoimidazole-5-carboxylic acid [2-(4-methyl-piperazin-1-yl)-2-oxo-ethyl]- |
| | amide |
| 191 | 1-Ethyl-2-[6-(pyridin-3-yloxy)-benzothiazol-2-ylamino]-1H- |
| | benzoimidazole-5-carboxylic acid (2-methoxy-ethyl)-amide |
| 192 | 1-Methyl-2-(6-trifluoromethoxy-benzothiazol-2-ylamino)-1H- |
| | benzoimidazole-5-carboxylic acid [2-(4-hydroxy-piperidin-1-yl)-ethyl]- |
| | amide |
| 193 | 1-Methyl-2-(6-trifluoromethoxy-benzothiazol-2-ylamino)-1H- |
| | benzoimidazole-5-carboxylic acid [2-(3-hydroxy-piperidin-1-yl)-ethyl]- |
| | amide |
| 194 | 1-Methyl-2-(6-trifluoromethoxy-benzothiazol-2-ylamino)-1H- |
| | benzoimidazole-5-carbonitrile |
| 195 | 1-Methyl-2-(6-trifluoromethoxy-benzothiazol-2-ylamino)-1H- |
| | benzoimidazole-6-carbonitrile |
| 196 | [5-(1H-Imidazol-2-yl)-1-methyl-1H-benzimidazol-2-yl]-(6- |
| | trifluoromethoxy-benzothiazol-2-yl)-amine |
| 197 | [1-Methyl-6-(1H-1,2,4-triazol-3-yl)-1H-benzimidazol-2-yl]-(6- |
| | trifluoromethoxy-benzothiazol-2-yl)-amine |
| 198 | [1-Methyl-6-(5-methyl-1H-1,2,4-triazol-3-yl)-1H-benzimidazol-2-yl]-(5- |
| | trifluoromethoxy-benzothiazol-2-yl)-amine |
| 199 | (1-Ethyl-5-trifluoromethanesulfonyl-1H-benzoimidazol-2-yl)-(6- |
| | trifluoromethoxy-benzothiazol-2-yl)-amine |
| 200 | 1-[1-Methyl-2-(6-trifluoromethoxy-benzothiazol-2-ylamino)-1H- |
| | benzoimidazol-5-yl]-ethanone |
| 201 | (5-Methanesulfonyl-1-methyl-1H-benzoimidazol-2-yl)-(6-trifluoromethoxy- |
| | benzothiazol-2-yl)-amine |
| 202 | 2-[1-Methyl-2-(6-trifluoromethoxy-benzothiazol-2-ylamino)-1H- |
| | benzoimidazol-6-yl]-acetamide |
| 203 | 2-(6-Chloro-benzothiazol-2-ylamino)-1-methyl-1H-benzoimidazole-5- |
| | carboxylic acid ((R)-2-hydroxy-propyl)-amide |
| 204 | 2-(6-Chloro-benzothiazol-2-ylamino)-1-methyl-1H-benzoimidazole-5- |
| | carboxylic acid ((S)-2-hydroxy-propyl)-amide |
| 205 | 1-Methyl-2-(6-trifluoromethyl-benzothiazol-2-ylamino)-1H- |
| | benzoimidazole-5-carboxylic acid ((R)-2-hydroxy-propyl)-amide |
| 206 | 1-Methyl-2-(6-trifluoromethyl-benzothiazol-2-ylamino)-1H- |
| | benzoimidazole-5-carboxylic acid ((S)-2-hydroxy-propyl)-amide |
| 207 | 2-(6-Chloro-benzothiazol-2-ylamino)-1-methyl-1H-benzoimidazole-5- |
| | carboxylic acid (2-methoxy-2-methyl-propyl)-amide |
| 208 | 1-Methyl-2-(6-trifluoromethyl-benzothiazol-2-ylamino)-1H- |
| | benzoimidazole-5-carboxylic acid (2-methoxy-2-methyl-propyl)-amide |
| 209 | 2-(6-Chloro-benzothiazol-2-ylamino)-1-methyl-1H-benzoimidazole-5- |
| | carboxylic acid (2-fluoro-ethyl)-amide |
| 210 | 1-Methyl-2-(6-trifluoromethyl-benzothiazol-2-ylamino)-1H- |
| | benzoimidazole-5-carboxylic acid (2-fluoro-ethyl)-amide |
| 211 | 1-Methyl-2-(6-trifluoromethoxy-benzothiazol-2-ylamino)-1H- |
| | benzoimidazole-5-carboxylic acid cyanomethyl-amide |
| 212 | 1-Methyl-2-(6-trifluoromethoxy-benzothiazol-2-ylamino)-1H- |
| | benzoimidazole-5-carboxylic acid (2-cyano-ethyl)-amide |
| 213 | 2-(6-Chloro-benzothiazol-2-ylamino)-1-methyl-1H-benzoimidazole-5- |
| | carboxylic acid (2-cyano-ethyl)-amide |
| 214 | 2-(6-Chloro-benzothiazol-2-ylamino)-1-methyl-1H-benzoimidazole-5- |
| | carboxylic acid (3-hydroxy-propyl)-amide |
| 215 | 2-(6-Chloro-benzothiazol-2-ylamino)-1-methyl-1H-benzoimidazole-5- |
| | carboxylic acid (3-hydroxy-butyl)-amide |
| 216 | 1-Methyl-2-(6-trifluoromethyl-benzothiazol-2-ylamino)-1H- |
| | benzoimidazole-5-carboxylic acid (3-hydroxy-butyl)-amide |
| 217 | 1-Methyl-2-(6-trifluoromethoxy-benzothiazol-2-ylamino)-1H- |
| | benzoimidazole-5-carboxylic acid (3-hydroxy-2,2-dimethyl-propyl)-amide |
| 218 | 2-(6-Chloro-benzothiazol-2-ylamino)-1-methyl-1H-benzoimidazole-5- |
| | carboxylic acid (3-hydroxy-2,2-dimethyl-propyl)-amide |
| 219 | 1-Methyl-2-(6-trifluoromethoxy-benzothiazol-2-ylamino)-1H- |
| | benzoimidazole-5-carboxylic acid (4-hydroxy-butyl)-amide |
| 220 | 2-(6-Chloro-1H-benzoimidazol-2-ylamino)-1-methyl-1H-benzoimidazole-5- |
| | carboxylic acid (4-hydroxy-butyl)-amide |
| 221 | 1-Methyl-2-(6-trifluoromethyl-benzothiazol-2-ylamino)-1H- |
| | benzoimidazole-5-carboxylic acid (4-hydroxy-butyl)-amide |
| 222 | 6-Fluoro-1-methyl-2-(6-trifluoromethoxy-benzothiazol-2-ylamino)-1H- |
| | benzoimidazole-5-carboxylic acid (4-hydroxy-butyl)-amide |
| 223 | 1-Methyl-2-(6-trifluoromethoxy-benzothiazol-2-ylamino)-1H- |
| | benzoimidazole-5-carboxylic acid ((R)-4-hydroxy-3-methyl-butyl)-amide |
| 224 | 2-(6-Chloro-benzothiazol-2-ylamino)-1-methyl-1H-benzoimidazole-5- |
| | carboxylic acid ((R)-4-hydroxy-3-methyl-butyl)-amide |
| 225 | 2-(6-Chloro-benzothiazol-2-ylamino)-1-methyl-1H-benzoimidazole-5- |
| | carboxylic acid (trans-4-hydroxy-cyclohexyl)-amide |
| 226 | 1-Methyl-2-(6-trifluoromethoxy-benzothiazol-2-ylamino)-1H- |
| | benzoimidazole-5-carboxylic acid (5-hydroxy-pentyl)-amide |
| 227 | 2-(6-Chloro-benzothiazol-2-ylamino)-1-methyl-1H-benzoimidazole-5- |
| | carboxylic acid (5-hydroxy-pentyl)-amide |
| 228 | 1-Methyl-2-(6-trifluoromethyl-benzothiazol-2-ylamino)-1H- |
| | benzoimidazole-5-carboxylic acid (5-hydroxy-pentyl)-amide |
| 229 | 1-Methyl-2-(6-trifluoromethoxy-benzothiazol-2-ylamino)-1H- |
| | benzoimidazole-5-carboxylic acid (5-hydroxy-4,4-dimethyl-pentyl)-amide |
| 230 | 1-Methyl-2-(6-trifluoromethoxy-benzothiazol-2-ylamino)-1H- |
| | benzoimidazole-5-carboxylic acid 4-hydroxy-benzylamide |
| 231 | 1-Methyl-2-(6-trifluoromethoxy-benzothiazol-2-ylamino)-1H- |
| | benzoimidazole-5-carboxylic acid 3-hydroxy-4-methoxy-benzylamide |
| 232 | 1-Methyl-2-(6-trifluoromethoxy-benzothiazol-2-ylamino)-1H- |
| | benzoimidazole-5-carboxylic acid (trans-4-hydroxy-cyclohexylmethyl)- |
| | amide |
| 233 | 2-(6-Chloro-benzothiazol-2-ylamino)-1-methyl-1H-benzoimidazole-5- |
| | carboxylic acid (trans-4-hydroxy-cyclohexylmethyl)-amide |
| 234 | 1-Methyl-2-(6-trifluoromethyl-benzothiazol-2-ylamino)-1H- |
| | benzoimidazole-5-carboxylic acid (trans-4-hydroxy-cyclohexylmethyl)- |
| | amide |
| 235 | 2-(6-Chloro-benzothiazol-2-ylamino)-1-methyl-1H-benzoimidazole-5- |
| | carboxylic acid [2-(2-hydroxy-ethoxy)-ethyl]-amide |
| 236 | 1-Methyl-2-(6-trifluoromethyl-benzothiazol-2-ylamino)-1H- |
| | benzoimidazole-5-carboxylic acid [2-(2-hydroxy-ethoxy)-ethyl]-amide |
| 237 | 6-Fluoro-1-methyl-2-(6-trifluoromethoxy-benzothiazol-2-ylamino)-1H- |
| | benzoimidazole-5-carboxylic acid [2-(2-hydroxy-ethoxy)-ethyl]-amide |
| 238 | 3-Methyl-2-(6-trifluoromethoxy-benzothiazol-2-ylamino)-3H-imidazo[4,5- |
| | b]pyridine-6-carboxylic acid [2-(2-hydroxy-ethoxy)-ethyl]-amide |
| 239 | 1-(2-Methylamino-ethyl)-2-(6-trifluoromethoxy-benzothiazol-2 ylamino)- |
| | 1H-benzoimidazole-5-carboxylic acid[2-(2-hydroxy-ethoxy)-ethyl]-amide |
| | hydrochloride |
| 240 | 2-(6-Chloro-benzothiazol-2-ylamino)-1-(2-methylamino-ethyl)-1H- |
| | benzoimidazole-5-carboxylic acid [2-(2-hydroxy-ethoxy)-ethyl]-amide |
| | hydrochloride |
| 241 | 1-(2-Methoxy-ethyl)-2-(6-trifluoromethoxy-benzothiazol-2-ylamino)-1H- |
| | benzoimidazole-5-carboxylic acid [2-(2-hydroxy-ethoxy)-ethyl]-amide |
| 242 | 1-Methyl-2-(6-trifluoromethoxy-benzothiazol-2-ylamino)-1H- |
| | benzoimidazole-5-carboxylic acid [2-((R)-2-hydroxy-1-methyl-ethoxy)- |
| | ethyl]-amide |
| 243 | 2-(6-Chloro-benzothiazol-2-ylamino)-1-methyl-1H-benzoimidazole-5- |
| | carboxylic acid [2-((R)-2-hydroxy-1-methyl-ethoxy)-ethyl]-amide |
| 244 | 1-Methyl-2-(6-trifluoromethoxy-benzothiazol-2-ylamino)-1H- |
| | benzoimidazole-5-carboxylic acid [2-(2-hydroxy-propoxy)-ethyl]-amide |
| 245 | 1-Methyl-2-(6-trifluoromethoxy-benzothiazol-2-ylamino)-1H- |
| | benzoimidazole-5-carboxylic acid [2-(2-hydroxy-2-methyl-propoxy)-ethyl]- |
| | amide |
| 246 | 1-Methyl-2-(6-trifluoromethoxy-benzothiazol-2-ylamino)-1H- |
| | benzoimidazole-5-carboxylic acid [2-(3-hydroxy-propoxy)-ethyl]-amide |
| 247 | 1-Methyl-2-(6-trifluoromethoxy-benzothiazol-2-ylamino)-1H- |
| | benzoimidazole-5-carboxylic acid [2-(3-fluoro-propoxy)-ethyl]-amide |
| 248 | 2-(6-Chloro-benzothiazol-2-ylamino)-1-methyl-1H-benzoimidazole-5- |
| | carboxylic acid [2-(3-hydroxy-propoxy)-ethyl]-amide |
| 249 | 2-(6-Chloro-benzothiazol-2-ylamino)-1-methyl-1H-benzoimidazole-5- |
| | carboxylic acid [2-(3-fluoro-propoxy)-ethyl]-amide |
| 250 | 1-Methyl-2-(6-trifluoromethoxy-benzothiazol-2-ylamino)-1H- |
| | benzoimidazole-5-carboxylic acid [3-(2-hydroxy-ethoxy)-propyl]-amide |
| 251 | 1-Methyl-2-(6-trifluoromethoxy-benzothiazol-2-ylamino)-1H- |
| | benzoimidazole-5-carboxylic acid [2-(4-hydroxy-phenyl)-ethyl]-amide |
| 252 | 1-Methyl-2-(6-trifluoromethoxy-benzothiazol-2-ylamino)-1H- |
| | benzoimidazole-5-carboxylic acid [2-(3-hydroxy-phenyl)-ethyl]-amide |
| 253 | 1-Methyl-2-(6-trifluoromethoxy-benzothiazol-2-ylamino)-1H- |
| | benzoimidazole-5-carboxylic acid [2-(4-hydroxy-cyclohexyl)-ethyl]-amide |
| 254 | 1-Methyl-2-(6-trifluoromethoxy-benzothiazol-2-ylamino)-1H- |
| | benzoimidazole-5-carboxylic acid (trans-4-hydroxymethyl- |
| | cyclohexylmethyl)-amide |
| 255 | 2-(6-Chloro-benzothiazol-2-ylamino)-1-methyl-1H-benzoimidazole-5- |
| | carboxylic acid (trans-4-hydroxymethyl-cyclohexylmethyl)-amide |
| 256 | 1-Methyl-2-(6-trifluoromethoxy-benzothiazol-2-ylamino)-1H- |
| | benzoimidazole-5-carboxylic acid {2-[2-(2-hydroxy-ethoxy)-ethoxy]-ethyl}- |
| | amide |
| 257 | 1-Methyl-2-(6-trifluoromethyl-benzothiazol-2-ylamino)-1H- |
| | benzoimidazole-5-carboxylic acid [2-(2-fluoro-ethoxy)-ethyl]-amide |
| 258 | 1-Methyl-2-(6-trifluoromethoxy-benzothiazol-2-ylamino)-1H- |
| | benzoimidazole-5-carboxylic acid [2-(2,2-difluoro-ethoxy)-ethyl]-amide |
| 259 | 2-(6-Chloro-benzothiazol-2-ylamino)-1-methyl-1H-benzoimidazole-5- |
| | carboxylic acid [2-(2,2-difluoro-ethoxy)-ethyl]-amide |
| 260 | 1-Methyl-2-(6-trifluoromethoxy-benzothiazol-2-ylamino)-1H- |
| | benzoimidazole-5-carboxylic acid [2-(2-methoxy-ethoxy)-ethyl]-amide |
| 261 | 2-(6-Chloro-benzothiazol-2-ylamino)-1-methyl-1H-benzoimidazole-5- |
| | carboxylic acid [2-(2-methoxy-ethoxy)-ethyl]-amide |
| 262 | 1-Methyl-2-(6-trifluoromethyl-benzothiazol-2-ylamino)-1H- |
| | benzoimidazole-5-carboxylic acid [2-(2-methoxy-ethoxy)-ethyl]-amide |
| 263 | 1-Methyl-2-(6-trifluoromethoxy-benzothiazol-2-ylamino)-1H- |
| | benzoimidazole-5-carboxylic acid [2-(tetrahydro-pyran-2-yl)-ethyl]-amide |
| 264 | 1-Methyl-2-(6-trifluoromethoxy-benzothiazol-2-ylamino)-1H- |
| | benzoimidazole-5-carboxylic acid [2-(tetrahydro-pyran-4-yl)-ethyl]-amide |
| 265 | 1-Methyl-2-(6-trifluoromethoxy-benzothiazol-2-ylamino)-1H- |
| | benzoimidazole-5-carboxylic acid [2-(2-cyano-ethoxy)-ethyl]-amide |
| 266 | 2-(6-Chloro-benzothiazol-2-ylamino)-1-methyl-1H-benzoimidazole-5- |
| | carboxylic acid [2-(2-cyano-ethoxy)-ethyl]-amide |
| 267 | 1-Methyl-2-(6-trifluoromethoxy-benzothiazol-2-ylamino)-1H- |
| | benzoimidazole-5-carboxylic acid (2-carbamoylmethoxy-ethyl)-amide |
| 268 | 1-Methyl-2-(6-trifluoromethoxy-benzothiazol-2-ylamino)-1H |
| | benzoimidazole-5-carboxylic acid [2-(2-amino-ethoxy)-ethyl]-amide |
| 269 | 2-(6-Chloro-benzothiazol-2-ylamino)-1-methyl-1H-benzoimidazole-5- |
| | carboxylic acid [2-(2-amino-ethoxy)-ethyl]-amide |
| 270 | 2-(4-Chloro-benzothiazol-2-ylamino)-1-methyl-1H benzoimidazole-5- |
| | carboxylic acid [2-(2-amino-ethoxy)-ethyl]-amide |
| 271 | 1-Methyl-2-(6-trifluoromethoxy-benzothiazol-2-ylamino)-1H- |
| | benzoimidazole-5-carboxylic acid [2-(2-methylamino-ethoxy)-ethyl]-amide |
| | hydrochloride |
| 272 | 2-(6-Chloro-benzothiazol-2-ylamino)-1-methyl-1H-benzoimidazole-5- |
| | carboxylic acid [2-(2-methylamino-ethoxy)-ethyl]-amide hydrochloride |
| 273 | 1-Methyl-2-(6-trifluoromethoxy-benzothiazol-2-ylamino)-1H- |
| | benzoimidazole-5-carboxylic acid [2-(2-dimethylamino-ethoxy)-ethyl]- |
| | amide |
| 274 | 2-(6-Chloro-benzothiazol-2-ylamino)-1-methyl-1H-benzoimidazole-5- |
| | carboxylic acid [2-(2-dimethylamino-ethoxy)-ethyl]-amide |
| 275 | 1-Methyl-2-(6-trifluoromethoxy-benzothiazol-2-ylamino)-1H- |
| | benzoimidazole-5-carboxylic acid [2-(2-acetylamino-ethoxy)-ethyl]-amide |
| 276 | 1-Methyl-2-(6-trifluoromethoxy-benzothiazol-2-ylamino)-1H- |
| | benzoimidazole-5-carboxylic acid [2-(2-methanesulfonylamino-ethoxy)- |
| | ethyl]-amide |
| 277 | 2-(6-Chloro-benzothiazol-2-ylamino)-1-methyl-1H-benzoimidazole-5- |
| | carboxylic acid (2-ethanesulfonyl-ethyl)-amide |
| 278 | 2-(6-Chloro-benzothiazol-2-ylamino)-1-methyl-1H-benzoimidazole-5- |
| | carboxylic acid [2-(2-hydroxy-ethanesulfonyl)-ethyl]-amide |
| 279 | 2-(6-Chloro-benzothiazol-2-ylamino)-1-methyl-1H-benzoimidazole-5- |
| | carboxylic acid [2-(2-fluoro-ethylamino)-ethyl]-amide hydrochloride |
| 280 | 1-Methyl-2-(6-trifluoromethoxy-benzothiazol-2-ylamino)-1H- |
| | benzoimidazole-5-carboxylic acid ((S)-2,3-dihydroxy-propyl)-amide |
| 281 | 1-Methyl-2-(6-trifluoromethoxy-benzothiazol-2-ylamino)-1H- |
| | benzoimidazole-5-carboxylic acid ((R)-2,3-dihydroxy-propyl)-amide |
| 282 | 1-Methyl-2-(6-trifluoromethoxy-benzothiazol-2-ylamino)-1H- |
| | benzoimidazole-5-carboxylic acid ((1R,2S,3R,4R)-2,3-dihydroxy-4- |
| | hydroxymethyl-cyclopentyl)-amide |
| 283 | 1-Methyl-2-(6-trifluoromethoxy-benzothiazol-2-ylamino)-1H- |
| | benzoimidazole-5-carboxylic acid ((2S,3R,4R,5S,6R)-2,4,5-trihydroxy-6- |
| | hydroxymethyl-tetrahydro-pyran-3-yl)-amide |
| 284 | 1-Methyl-2-(6-trifluoromethoxy-benzothiazol-2-ylamino)-1H- |
| | benzoimidazole-5-carboxylic acid bis-(2-hydroxy-ethyl)-amide |
| 285 | 3-Methyl-2-(6-trifluoromethoxy-benzothiazol-2-ylamino)-3H- |
| | benzoimidazole-5-carboxylic acid (4-hydroxy-butyl)-amide |
| 286 | 3-Methyl-2-(6-trifluoromethoxy-benzothiazol-2-ylamino)-3H- |
| | benzoimidazole-5-carboxylic acid [2-(2-hydroxy-ethoxy)-ethyl]-amide |
| 287 | 2-(6-Chloro-benzothiazol-2-ylamino)-3-methyl-3H-benzoimidazole-5- |
| | carboxylic acid (4-hydroxy-butyl)-amide |
| 288 | 2-(6-Chloro-benzothiazol-2-ylamino)-3-methyl-3H-benzoimidazole-5- |
| | carboxylic acid [2-(2-hydroxy-ethoxy)-ethyl]-amide |
| 289 | 1-Methyl-2-(6-trifluoromethoxy-benzothiazol-2-ylamino)-1H- |
| | benzoimidazole-5-carboxylic acid pyrrolidin-3-ylamide hydrochloride |
| 290 | 2-(6-Chloro-benzothiazol-2-ylamino)-1-methyl-1H-benzoimidazole-5- |
| | carboxylic acid (R)-pyrrolidin-3-ylamide hydrochloride |
| 291 | 2-(6-Chloro-benzothiazol-2-ylamino)-1-methyl-1H-benzoimidazole-5- |
| | carboxylic acid (S)-pyrrolidin-3-ylamide hydrochloride |
| 292 | 2-(6-Chloro-benzothiazol-2-ylamino)-1-methyl-1H-benzoimidazole-5- |
| | carboxylic acid [(R)-1-(2-hydroxy-ethyl)-pyrrolidin-3-yl]-amide |
| 293 | 2-(6-Chloro-benzothiazol-2-ylamino)-1-methyl-1H-benzoimidazole-5- |
| | carboxylic acid [(S)-1-(2-hydroxy-ethyl)-pyrrolidin-3-yl]-amide |
| 294 | 1-Methyl-2-(6-trifluoromethoxy-benzothiazol-2-ylamino)-1H- |
| | benzoimidazole-5-carboxylic acid [1-((R)-2-hydroxy-propyl)-pyrrolidin-3- |
| | yl]-amide |
| 295 | 2-(6-Chloro-benzothiazol-2-ylamino)-1-methyl-1H-benzoimidazole-5- |
| | carboxylic acid [(R)-1-((S)-2-hydroxy-propionyl)-pyrrolidin-3-yl]-amide |
| 296 | 2-(6-Chloro-benzothiazol-2-ylamino)-1-methyl-1H-benzoimidazole-5- |
| | carboxylic acid [(R)-1-((R)-2-hydroxy-propyl)-pyrrolidin-3-yl]-amide |
| 297 | 1-Methyl-2-(6-trifluoromethyl-benzothiazol-2-ylamino)-1H- |
| | benzoimidazole-5-carboxylic acid [(R)-1-((R)-2-hydroxy-propyl)-pyrrolidin- |
| | 3-yl]-amide |
| 298 | 1-Methyl-2-(6-trifluoromethoxy-benzothiazol-2-ylamino)-1H- |
| | benzoimidazole-5-carboxylic acid [1-(2-hydroxy-2-methyl-propyl)- |
| | pyrrolidin-3-yl]-amide |
| 299 | 3-(3-{[2-(6-Chloro-benzothiazol-2-ylamino)-1-methyl-1H-benzoimidazole-5- |
| | carbonyl]-amino}-pyrrolidin-1-yl)-propionic acid |
| 300 | 2-(6-Chloro-benzothiazol-2-ylamino)-1-methyl-1H-benzoimidazole-5- |
| | carboxylic acid [1-(2-methanesulfonylamino-ethyl)-pyrrolidin-3-yl]-amide |
| 301 | 2-(6-Chloro-benzothiazol-2-ylamino)-1-(2-methoxy-ethyl)-1H- |
| | benzoimidazole-5-carboxylic acid [1-(2-hydroxy-ethyl)-piperidin-4-yl]- |
| | amide |
| 302 | 2-(6-Chloro-benzothiazol-2-ylamino)-1-methyl-1H-benzoimidazole-5- |
| | carboxylic acid (piperidin-4-ylmethyl)-amide hydrochloride |
| 303 | 2-(6-Chloro-benzothiazol-2-ylamino)-1-methyl-1H-benzoimidazole-5- |
| | carboxylic acid [1-(2-hydroxy-ethyl)-piperidin-4-ylmethyl]-amide |
| 304 | 2-(6-Chloro-benzothiazol-2-ylamino)-1-(2-methoxy-ethyl)-1H- |
| | benzoimidazole-5-carboxylic acid [1-(2-hydroxy-ethyl)-piperidin-4- |
| | ylmethyl]-amide |
| 305 | [4-(2-Hydroxy-ethyl)-piperazin-1-yl]-[1-methyl-2-(6-trifluoromethoxy- |
| | benzothiazol-2-ylamino)-1H-benzoimidazol-5-yl]-methanone |
| 306 | [2-(6-Chloro-benzothiazol-2-ylamino)-1-methyl-1H-benzoimidazol-5-yl]-[4- |
| | (2-hydroxy-ethyl)-piperazin-1-yl]-methanone |
| 307 | [4-(3-Hydroxy-propyl)-piperidin-1-yl]-[1-methyl-2-(6-trifluoromethoxy- |
| | benzothiazol-2-ylamino)-1H benzoimidazol-5-yl]-methanone |
| 308 | 2-(6-Chloro-benzothiazol-2-ylamino)-1-methyl-1H-benzoimidazole-5- |
| | carboxylic acid [(R)-1-(2-dimethylamino-acetyl)-pyrrolidin-3-yl]-amide |
| 309 | 2-(6-Chloro-benzothiazol-2-ylamino)-1-methyl-1H-benzoimidazole-5- |
| | carboxylic acid (R)-piperidin-3-ylamide hydrochloride |
| 310 | 2-(6-Chloro-benzothiazol-2-ylamino)-1-methyl-1H-benzoimidazole-5- |
| | carboxylic acid (S)-piperidin-3-ylamide hydrochloride |
| 311 | 2-(6-Chloro-benzothiazol-2-ylamino)-1-methyl-1H-benzoimidazole-5- |
| | carboxylic acid [(R)-1-(2-dimethylamino-acetyl)-piperidin-3-yl]-amide |
| 312 | 2-(6-Chloro-benzothiazol-2-ylamino)-1-methyl-1H-benzoimidazole-5- |
| | carboxylic acid [(S)-1-(2-dimethylamino-acetyl)-piperidin-3-yl]-amide |
| 313 | 2-(6-Chloro-benzothiazol-2-ylamino)-1-methyl-1H-benzoimidazole-5- |
| | carboxylic acid [1-(2-dimethylamino-acetyl)-piperidin-4-yl]-amide |
| 314 | 2-(6-Chloro-benzothiazol-2-ylamino)-1-methyl-1H-benzoimidazole-5- |
| | carboxylic acid [1-(2-dimethylamino-acetyl)-piperidin-4-ylmethyl]-amide |
| 315 | 2-(6-Chloro-benzothiazol-2-ylamino)-1-(2-methoxy-ethyl)-1H- |
| | benzoimidazole-5-carboxylic acid [1-(2-dimethylamino-acetyl)-piperidin-4- |
| | ylmethyl]-amide |
| 316 | 2-(6-Chloro-benzothiazol-2-ylamino)-1-methyl-1H benzoimidazole-5- |
| | carboxylic acid ((R)-1-methyl-pyrrolidin-3-yl)-amide |
| 317 | 2-(6-Chloro-benzothiazol-2 ylamino)-1-methyl-1H-benzoimidazole-5- |
| | carboxylic acid ((S)-1-methyl-pyrrolidin-3-yl)-amide |
| 318 | 2-(6-Chloro-benzothiazol-2-ylamino)-1-methyl-1H-benzoimidazole-5- |
| | carboxylic acid (1-methyl-piperidin-2-ylmethyl)-amide |
| 319 | 2-(6-Chloro-benzothiazol-2-ylamino)-1-methyl-1H-benzoimidazole-5- |
| | carboxylic acid (1-methyl-piperidin-4-yl)-amide |
| 320 | 2-(6-Chloro-benzothiazol-2-ylamino)-1-methyl-1H-benzoimidazole-5- |
| | carboxylic acid (1-methanesulfonyl-piperidin-4-yl)-amide |
| 321 | 2-(6-Chloro-benzothiazol-2-ylamino)-1-methyl-1H-benzoimidazole-5- |
| | carboxylic acid morpholin-4-ylamide |
| 322 | 1-Methyl-2-(6-trifluoromethoxy-benzothiazol-2-ylamino)-1H- |
| | benzoimidazole-5-carboxylic acid (2-methanesulfonylamino-ethyl)-amide |
| 323 | 1-Methyl-2-(6-trifluoromethoxy-benzothiazol-2-ylamino)-1H- |
| | benzoimidazole-5-carboxylic acid [2-(2-dimethylamino-acetylamino)- |
| | ethyl]-amide |
| 324 | 2-(6-Chloro-benzothiazol-2-ylamino)-1-methyl-1H-benzoimidazole-5- |
| | carboxylic acid [2-(2-dimethylamino-acetylamino)-ethyl]-amide |
| 325 | 1-Methyl-2-(6-trifluoromethoxy-benzothiazol-2-ylamino)-1H- |
| | benzoimidazole-5-carboxylic acid [2-(2-hydroxy-acetylamino)-ethyl]-amide |
| 326 | 1-Methyl-2-(6-trifluoromethoxy-benzothiazol-2-ylamino)-1H- |
| | benzoimidazole-5-carboxylic acid [2-((S)-2-hydroxy-propionylamino)- |
| | ethyl]-amide |
| 327 | 1-Methyl-2-(6-trifluoromethoxy-benzothiazol-2-ylamino)-1H- |
| | benzoimidazole-5-carboxylic acid (2-imidazol-1-yl-ethyl)-amide |
| 328 | 1-Methyl-2-(6-trifluoromethoxy-benzothiazol-2-ylamino)-1H- |
| | benzoimidazole-5-carboxylic acid (2-pyrazol-1-yl-ethyl)-amide |
| 329 | 1-Methyl-2-(6-trifluoromethoxy-benzothiazol-2-ylamino)-1H- |
| | benzoimidazole-5-carboxylic acid [2-(2-oxo-pyrrolidin-1-yl)-ethyl]-amide |
| 330 | 1-Methyl-2-(6-trifluoromethoxy-benzothiazol-2-ylamino)-1H- |
| | benzoimidazole-5-carboxylic acid [2-(2-oxo-imidazolidin-1-yl)-ethyl]-amide |
| 331 | 2-(6-Chloro-benzothiazol-2-ylamino)-1-methyl-1H-benzoimidazole-5- |
| | carboxylic acid [2-(3-oxo-piperazin-1-yl)-ethyl]-amide |
| 332 | 2-(6-Chloro-benzothiazol-2-ylamino)-1-(2-methoxy-ethyl)-1H- |
| | benzoimidazole-5-carboxylic acid [2-(3-oxo-piperazin-1-yl)-ethyl]-amide |
| 333 | 1-Methyl-2-(6-trifluoromethoxy-benzothiazol-2-ylamino)-1H- |
| | benzoimidazole-5-carboxylic acid (2-piperidin-1-yl-ethyl)-amide |
| 334 | 2-(6-Chloro-benzothiazol-2-ylamino)-1-methyl-1H-benzoimidazole-5- |
| | carboxylic acid (2-piperidin-1-yl-ethyl)-amide |
| 335 | 2-(6-Chloro-benzothiazol-2-ylamino)-1-methyl-1H-benzoimidazole-5- |
| | carboxylic acid [2-(4,4-difluoro-piperidin-1-yl)-ethyl]-amide |
| 336 | 2-(6-Chloro-benzothiazol-2-ylamino)-1-methyl-1H-benzoimidazole-5- |
| | carboxylic acid [2-(3,3-difluoro-piperidin-1-yl)-ethyl]-amide |
| 337 | 1-Methyl-2-(6-trifluoromethoxy-benzothiazol-2-ylamino)-1H- |
| | benzoimidazole-5-carboxylic acid [2-(4-methyl-piperazin-1-yl)-ethyl]- |
| | amide |
| 338 | 2-(6-Chloro-benzothiazol-2-ylamino)-1-methyl-1H-benzoimidazole-5- |
| | carboxylic acid [2-(4-methyl-piperazin-1-yl)-ethyl]-amide |
| 339 | 2-(6-Chloro-benzothiazol-2-ylamino)-1-methyl-1H-benzoimidazole-5- |
| | carboxylic acid (2-piperazin-1-yl-ethyl)-amide hydrochloride |
| 340 | 2-(6-Chloro-benzothiazol-2-ylamino)-1-methyl-1H-benzoimidazole-5- |
| | carboxylic acid (2-morpholin-4-yl-ethyl)-amide |
| 341 | 1-Methyl-2-(6-trifluoromethyl-benzothiazol-2-ylamino)-1H- |
| | benzoimidazole-5-carboxylic acid (2-morphobn-4-yl-ethyl)-amide |
| 342 | 1-Methyl-2-(6-trifluoromethoxy-benzothiazol-2-ylamino)-1H- |
| | benzoimidazole-5-carboxylic acid [2-(1,1-dioxo-thiomorpholin-4-yl)-ethyl]- |
| | amide |
| 343 | 2-(6-Chloro-benzothiazol-2-ylamino)-1-methyl-1H-benzoimidazole-5- |
| | carboxylic acid (2-amino-ethyl)-amide hydrochloride |
| 344 | 1-Methyl-2-(6-trifluoromethoxy-benzothiazol-2-ylamino)-1H- |
| | benzoimidazole-5-carboxylic acid (3-amino-propyl)-amide hydrochloride |
| 345 | 2-(6-Chloro-benzothiazol-2-ylamino)-1-methyl-1H-benzoimidazole-5- |
| | carboxylic acid (3-amino-propyl)-amide hydrochloride |
| 346 | 1-Methyl-2-(6-trifluoromethoxy-benzothiazol-2-ylamino)-1H- |
| | benzoimidazole-5-carboxylic acid (4-amino-butyl)-amide hydrochloride |
| 347 | 2-(6-Chloro-benzothiazol-2-ylamino)-1-methyl-1H-benzoimidazole-5- |
| | carboxylic acid (4-amino-butyl)-amide hydrochloride |
| 348 | 1-Methyl-2-(6-trifluoromethoxy-benzothiazol-2-ylamino)-1H- |
| | benzoimidazole-5-carboxylic acid (3-dimethylamino-propyl)-amide |
| 349 | 2-(6-Chloro-benzothiazol-2-ylamino)-1-methyl-1H-benzoimidazole-5- |
| | carboxylic acid (3-dimethylamino-propyl)-amide |
| 350 | 1-Methyl-2-(6-trifluoromethyl-benzothiazol-2-ylamino)-1H- |
| | benzoimidazole-5-carboxylic acid (3-dimethylamino-propyl)-amide |
| 351 | 1-Methyl-2-(6-trifluoromethoxy-benzothiazol-2-ylamino)-1H- |
| | benzoimidazole-5-carboxylic acid (3-diethylamino-propyl)-amide |
| 352 | 2-(6-Chloro-benzothiazol-2-ylamino)-1-methyl-1H-benzoimidazole-5- |
| | carboxylic acid (3-diethylamino-propyl)-amide |
| 353 | 1-Methyl-2-(6-trifluoromethyl-benzothiazol-2-ylamino)-1H- |
| | benzoimidazole-5-carboxylic acid (3-diethylamino-propyl)-amide |
| 354 | 2-(6-Chloro-benzothiazol-2-ylamino)-1-methyl-1H-benzoimidazole-5- |
| | carboxylic acid (3-pyrrolidin-1-yl-propyl)-amide |
| 355 | 1-Methyl-2-(6-trifluoromethoxy-benzothiazol-2-ylamino)-1H- |
| | benzoimidazole-5-carboxylic acid [3-(4-methyl-piperazin-1-yl)-propyl]- |
| | amide |
| 356 | 2-(6-Chloro-benzothiazol-2-ylamino)-1-methyl-1H-benzoimidazole-5- |
| | carboxylic acid [3-(4-methyl-piperazin-1-yl)-propyl]-amide |
| 357 | 1-Methyl-2-(6-trifluoromethyl-benzothiazol-2-ylamino)-1H- |
| | benzoimidazole-5-carboxylic acid [3-(4-methyl-piperazin-1-yl)-propyl]- |
| | amide |
| 358 | 1-Methyl-2-(6-trifluoromethoxy-benzothiazol-2-ylamino)-1H- |
| | benzoimidazole-5-carboxylic acid (3-morpholin-4-yl-propyl)-amide |
| 359 | 2-(6-Chloro-benzothiazol-2-ylamino)-1-methyl-1H-benzoimidazole-5- |
| | carboxylic acid (3-morpholin-4-yl-propyl)-amide |
| 360 | 1-Methyl-2-(6-trifluoromethoxy-benzothiazol-2-ylamino)-1H- |
| | benzoimidazole-5-carboxylic acid (4-diethylamino-butyl)-amide |
| 361 | 6-Diethylamino-1-methyl-2-(6-trifluoromethoxy-benzothiazol-2-ylamino)- |
| | 1H-benzoimidazole-5-carboxylic acid dimethylcarbamoylmethyl-amide |
| 362 | 6-Diethylamino-1-methyl-2-(6-trifluoromethoxy-benzothiazol-2-ylamino)- |
| | 1H-benzoimidazole-5-carboxylic acid (2-morpholin-4-yl-ethyl)-amide |
| 363 | 1-Methyl-2-(6-methyl-benzothiazol-2-ylamino)-1H-benzoimidazole-5- |
| | carboxylic acid dimethylcarbamoylmethyl-amide |
| 364 | 2-(6-Ethoxy-benzothiazol-2-ylamino)-1-methyl-1H-benzoimidazole-5- |
| | carboxylic acid dimethylcarbamoylmethyl-amide |
| 365 | 2-(6-Isopropyl-benzothiazol-2-ylamino)-1-methyl-1H-benzoimidazole-5- |
| | carboxylic acid dimethylcarbamoylmethyl-amide |
| 366 | 2-(6-Chloro-benzothiazol-2-ylamino)-1-(2-methoxy-ethyl)-1H- |
| | benzoimidazole-5-carboxylic acid dimethylcarbamoylmethyl-amide |
| 367 | 2-(6-Chloro-benzothiazol-2-ylamino)-1-(2-methylamino-ethyl)-1H- |
| | benzoimidazole-5-carboxylic acid dimethylcarbamoylmethyl-amide |
| | hydrochloride |
| 368 | 1-Methyl-2-(6-trifluoromethoxy-benzothiazol-2-ylamino)-1H- |
| | benzoimidazole-5-carboxylic acid ((S)-1-dimethylcarbamoyl-2-hydroxy- |
| | ethyl)-amide |
| 369 | 1-Methyl-2-(6-trifluoromethyl-benzothiazol-2-ylamino)-1H- |
| | benzoimidazole-5-carboxylic acid ((S)-5-amino-1-dimethylcarbamoyl- |
| | pentyl)-amide hydrochloride |
| 370 | 1-Methyl-2-(6-trifluoromethyl-benzothiazol-2-ylamino)-1H- |
| | benzoimidazole-5-carboxylic acid ((S)-5-dimethylamino-1- |
| | dimethylcarbamoyl-pentyl)-amide |
| 371 | 2-(6-Chloro-benzothiazol-2-ylamino)-1-methyl-1H-benzoimidazole-5- |
| | carboxylic acid (2-dimethylcarbamoyl-ethyl)-amide |
| 372 | 1-Methyl-2-(6-trifluoromethyl-benzothiazol-2-ylamino)-1H- |
| | benzoimidazole-5-carboxylic acid (2-dimethylcarbamoyl-ethyl)-amide |
| 373 | 2-(6-Chloro-benzothiazol-2-ylamino)-1-methyl-1H-benzoimidazole-5- |
| | carboxylic acid (3-morpholin-4-yl-3-oxo-propyl)-amide |
| 374 | 1-Methyl-2-(6-trifluoromethyl-benzothiazol-2-ylamino)-1H- |
| | benzoimidazole-5-carboxylic acid (3-morpholin-4-yl-3-oxo-propyl)-amide |
| 375 | 1-Methyl-2-(6-trifluoromethyl-benzothiazol-2-ylamino)-1H- |
| | benzoimidazole-5-carboxylic acid [3-(4-methyl-piperazin-1-yl)-3-oxo- |
| | propyl]-amide |
| 376 | 2-(6-Chloro-benzothiazol-2-ylamino)-1-methyl-1H-benzoimidazole-5- |
| | carboxylic acid (3-dimethylcarbamoyl-propyl)-amide |
| 377 | 2-(6-Chloro-benzothiazol-2-ylamino)-1-methyl-1H-benzoimidazole-5- |
| | carboxylic acid [4-(4-methyl-piperazin-1-yl)-4-oxo-butyl]-amide |
| 378 | 4-{[2-(6-Chloro-benzothiazol-2-ylamino)-1-methyl-1H-benzoimidazole-5- |
| | carbonyl]-amino}-trans-cyclohexanecarboxylic acid |
| 379 | 2-(6-Chloro-benzothiazol-2-ylamino)-1-methyl-1H-benzoimidazole-5- |
| | carboxylic acid (4-trans-dimethylcarbamoyl-cyclohexyl)-amide |
| 380 | 2-(6-Chloro-benzothiazol-2-ylamino)-1-methyl-1H-benzoimidazole-5- |
| | carboxylic acid methylcarbamoylmethyl-amide |
| 381 | 1-Methyl-2-(6-trifluoromethyl-benzothiazol-2-ylamino)-1H- |
| | benzoimidazole-5-carboxylic acid methylcarbamoylmethyl-amide |
| 382 | 2-(6-Chloro-benzothiazol-2-ylamino)-1-methyl-1H-benzoimidazole-5- |
| | carboxylic acid [((R)-2-hydroxy-propylcarbamoyl)-methyl]-amide |
| 383 | 2-(6-Chloro-benzothiazol-2-ylamino)-1-methyl-1H-benzoimidazole-5- |
| | carboxylic acid [(2-methanesulfonyl-ethylcarbamoyl)-methyl]-amide |
| 384 | 2-(6-Chloro-benzothiazol-2-ylamino)-1-methyl-1H-benzoimidazole-5- |
| | carboxylic acid [(tetrahydro-furan-3-ylcarbamoyl)-methyl]-amide |
| 385 | 2-(6-Chloro-benzothiazol-2-ylamino)-1-methyl-1H-benzoimidazole-5- |
| | carboxylic acid [(tetrahydro-pyran-4-ylcarbamoyl)-methyl]-amide |
| 386 | 2-(6-Chloro-benzothiazol-2-ylamino)-1-methyl-1H-benzoimidazole-5- |
| | carboxylic acid [(1-methyl-piperidin-4-ylcarbamoyl)-methyl]-amide |
| 387 | 2-(6-Chloro-benzothiazol-2-ylamino)-1-methyl-1H-benzoimidazole-5- |
| | carboxylic acid ((R)-piperidin-3-ylcarbamoylmethyl)-amide hydrochloride |
| 388 | 2-(6-Chloro-benzothiazol-2-ylamino)-1-methyl-1H-benzoimidazole-5- |
| | carboxylic acid [((R)-1-methyl-piperidin-3-ylcarbamoyl)-methyl]-amide |
| 389 | 1-Methyl-2-(6-trifluoromethoxy-benzothiazol-2-ylamino)-1H- |
| | benzoimidazole-5-carboxylic acid [(4-hydroxy-benzylcarbamoyl)-methyl]- |
| | amide |
| 390 | 1-Methyl-2-(6-trifluoromethoxy-benzothiazol-2-ylamino)-1H- |
| | benzoimidazole-5-carboxylic acid {[(2-hydroxy-ethyl)-methyl-carbamoyl]- |
| | methyl}-amide |
| 391 | 2-(6-Chloro-benzothiazol-2-ylamino)-1-methyl-1H-benzoimidazole-5- |
| | carboxylic acid {[(2-hydroxy-ethyl)-methyl-carbamoyl]-methyl}-amide |
| 392 | 1-Methyl-2-(6-trifluoromethoxy-benzothiazol-2-ylamino)-1H- |
| | benzoimidazole-5-carboxylic acid {[bis-(2-hydroxy-ethyl)-carbamoyl]- |
| | methyl}-amide |
| 393 | 2-(6-Chloro-benzothiazol-2-ylamino)-1-methyl-1H-benzoimidazole-5- |
| | carboxylic acid {[methyl-(tetrahydro-pyran-4-yl)-carbamoyl]-methyl}- |
| | amide |
| 394 | 2-(6-Chloro-benzothiazol-2-ylamino)-1-methyl-1H-benzoimidazole-5- |
| | carboxylic acid [(methyl-pyrrolidin-3-yl-carbamoyl)-methyl]-amide |
| | hydrochloride |
| 395 | 2-(6-Chloro-benzothiazol-2-ylamino)-1-methyl-1H-benzoimidazole-5- |
| | carboxylic acid {[methyl-(1-methyl-pyrrolidin-3-yl)-carbamoyl]-methyl}- |
| | amide |
| 396 | 2-(6-Chloro-benzothiazol-2-ylamino)-1-methyl-1H-benzoimidazole-5- |
| | carboxylic acid [(methyl-piperidin-3-yl-carbamoyl)-methyl]-amide |
| | hydrochloride |
| 397 | 2-(6-Chloro-benzothiazol-2-ylamino)-1-methyl-1H-benzoimidazole-5- |
| | carboxylic acid (2-oxo-2-pyrrolidin-1-yl-ethyl)-amide |
| 398 | 1-Methyl-2-(6-trifluoromethoxy-benzothiazol-2-ylamino)-1H- |
| | benzoimidazole-5-carboxylic acid [2-(3-hydroxy-pyrrolidin-1-yl)-2-oxo- |
| | ethyl]-amide |
| 399 | 2-(6-Chloro-benzothiazol-2-ylamino)-1-methyl-1H-benzoimidazole-5- |
| | carboxylic acid [2-(3-hydroxy-pyrrolidin-1-yl)-2-oxo-ethyl]-amide |
| 400 | 2-(6-Chloro-benzothiazol-2-ylamino)-1-methyl-1H-benzoimidazole-5- |
| | carboxylic acid [2-((R)-3-hydroxy-pyrrolidin-1-yl)-2-oxo-ethyl]-amide |
| 401 | 2-(6-Chloro-benzothiazol-2-ylamino)-1-methyl-1H-benzoimidazole-5- |
| | carboxylic acid [2-((S)-3-hydroxy-pyrrolidin-1-yl)-2-oxo-ethyl]-amide |
| 402 | 1-Methyl-2-(6-trifluoromethyl-benzothiazol-2-ylamino)-1H- |
| | benzoimidazole-5-carboxylic acid [2-((R)-3-hydroxy-pyrrolidin-1-yl)-2-oxo- |
| | ethyl]-amide |
| 403 | 1-Methyl-2-(6-trifluoromethyl-benzothiazol-2-ylamino)-1H- |
| | benzoimidazole-5-carboxylic acid [2-((S)-3-hydroxy-pyrrolidin-1-yl)-2-oxo- |
| | ethyl]-amide |
| 404 | 2-(6-Chloro-benzothiazol-2-ylamino)-1-(2-methoxy-ethyl)-1H- |
| | benzoimidazole-5-carboxylic acid [2-((R)-3-hydroxy-pyrrolidin-1-yl)-2-oxo- |
| | ethyl]-amide |
| 405 | 2-(6-Chloro-benzothiazol-2-ylamino)-1-(2-methoxy-ethyl)-1H- |
| | benzoimidazole-5-carboxylic acid [2-((S)-3-hydroxy-pyrrolidin-1-yl)-2-oxo- |
| | ethyl]-amide |
| 406 | 1-Methyl-2-(6-trifluoromethoxy-benzothiazol-2-ylamino)-1H- |
| | benzoimidazole-5-carboxylic acid [2-((S)-2-hydroxymethyl-pyrrolidin-1-yl)- |
| | 2-oxo-ethyl]-amide |
| 407 | 1-Methyl-2-(6-trifluoromethoxy-benzothiazol-2-ylamino)-1H- |
| | benzoimidazole-5-carboxylic acid [2-((3S,4S)-3,4-dihydroxy-pyrrolidin-1- |
| | yl)-2-oxo-ethyl]-amide |
| 408 | 2-(6-Chloro-benzothiazol-2-ylamino)-1-methyl-1H-benzoimidazole-5- |
| | carboxylic acid [2-((R)-3-methoxy-pyrrolidin-1-yl)-2-oxo-ethyl]-amide |
| 409 | 2-(6-Chloro-benzothiazol-2-ylamino)-1-methyl-1H-benzoimidazole-5- |
| | carboxylic acid [2-((S)-3-methoxy-pyrrolidin-1-yl)-2-oxo-ethyl]-amide |
| 410 | 1-Methyl-2-(6-trifluoromethyl-benzothiazol-2-ylamino)-1H- |
| | benzoimidazole-5-carboxylic acid [2-((S)-3-methoxy-pyrrolidin-1-yl)-2-oxo- |
| | ethyl]-amide |
| 411 | 2-(6-Chloro-benzothiazol-2-ylamino)-1-methyl-1H-benzoimidazole-5- |
| | carboxylic acid [2-((S)-3-amino-pyrrolidin-1-yl)-2-oxo-ethyl]-amide |
| | hydrochloride |
| 412 | 1-Methyl-2-(6-trifluoromethoxy-benzothiazol-2-ylamino)-1H- |
| | benzoimidazole-5-carboxylic acid [2-((S)-3-amino-pyrrolidin-1-yl)-2-oxo- |
| | ethyl]-amide hydrochloride |
| 413 | 1-Methyl-2-(6-trifluoromethoxy-benzothiazol-2-ylamino)-1H- |
| | benzoimidazole-5-carboxylic acid [2-((R)-3-methylamino-pyrrolidin-1-yl)-2- |
| | oxo-ethyl]-amide hydrochloride |
| 414 | 1-Methyl-2-(6-trifluoromethoxy-benzothiazol-2-ylamino)-1H- |
| | benzoimidazole-5-carboxylic acid [2-((S)-3-methylamino-pyrrolidin-1-yl)-2- |
| | oxo-ethyl]-amide hydrochloride |
| 415 | 1-Methyl-2-(6-trifluoromethoxy-benzothiazol-2-ylamino)-1H- |
| | benzoimidazole-5-carboxylic acid [2-((R)-3-dimethylamino-pyrrolidin-1-yl)- |
| | 2-oxo-ethyl]-amide |
| 416 | 1-Methyl-2-(6-trifluoromethoxy-benzothiazol-2-ylamino)-1H- |
| | benzoimidazole-5-carboxylic acid [2-((S)-3-dimethylamino-pyrrolidin-1-yl)- |
| | 2-oxo-ethyl]-amide |
| 417 | 2-(6-Chloro-benzothiazol-2-ylamino)-1-methyl-1H-benzoimidazole-5- |
| | carboxylic acid [2-((R)-3-dimethylamino-pyrrolidin-1-yl)-2-oxo-ethyl]- |
| | amide |
| 418 | 2-(6-Chloro-benzothiazol-2-ylamino)-1-methyl-1H-benzoimidazole-5- |
| | carboxylic acid [2-((S)-3-dimethylamino-pyrrolidin-1-yl)-2-oxo-ethyl]- |
| | amide |
| 419 | 2-(6-Chloro-benzothiazol-2-ylamino)-1-methyl-1H-benzoimidazole-5- |
| | carboxylic acid [2-((S)-2-dimethylcarbamoyl-pyrrolidin-1-yl)-2-oxo-ethyl]- |
| | amide |
| 420 | 3-Methyl-2-(6-trifluoromethoxy-benzothiazol-2-ylamino)-3H-imidazo[4,5- |
| | b]pyridine-6-carboxylic acid (2-morpholin-4-yl-2-oxo-ethyl)-amide |
| 421 | 2-(6-Chloro-benzothiazol-2-ylamino)-1-methyl-1H-benzoimidazole-5- |
| | carboxylic acid (2-morpholin-4-yl-2-oxo-ethyl)-amide |
| 422 | 1-Methyl-2-(6-trifluoromethyl-benzothiazol-2-ylamino)-1H- |
| | benzoimidazole-5-carboxylic acid (2-morpholin-4-yl-2-oxo-ethyl)-amide |
| 423 | 2-(6-Chloro-benzothiazol-2-ylamino)-1-methyl-1H-benzoimidazole-5- |
| | carboxylic acid (2-oxo-2-thiomorpholin-4-yl-ethyl)-amide |
| 424 | 2-(6-Chloro-benzothiazol-2-ylamino)-1-methyl-1H-benzoimidazole-5- |
| | carboxylic acid [2-(1,1-dioxo-thiomorpholin-4-yl)-2-oxo-ethyl]-amide |
| 425 | 2-(6-Chloro-benzothiazol-2-ylamino)-1-methyl-1H-benzoimidazole-5- |
| | carboxylic acid (2-oxo-2-piperazin-1-yl-ethyl)-amide hydrochloride |
| 426 | 2-(6-Chloro-benzothiazol-2-ylamino)-1-methyl-1H-benzoimidazole-5- |
| | carboxylic acid [2-(4-methyl-piperazin-1-yl)-2-oxo-ethyl]-amide |
| 427 | 1-Methyl-2-(6-trifluoromethyl-benzothiazol-2-ylamino)-1H- |
| | benzoimidazole-5-carboxylic acid [2-(4-methyl-piperazin-1-yl)-2-oxo-ethyl]- |
| | amide |
| 428 | 2-(6-Chloro-benzothiazol-2-ylamino)-1-(2-methoxy-ethyl)-1H- |
| | benzoimidazole-5-carboxylic acid [2-(4-methyl-piperazin-1-yl)-2-oxo-ethyl]- |
| | amide |
| 429 | 2-(6-Chloro-benzothiazol-2-ylamino)-1-methyl-1H-benzoimidazole-5- |
| | carboxylic acid [2-(4-methanesulfonyl-piperazin-1-yl)-2-oxo-ethyl]-amide |
| 430 | 2-(6-Chloro-benzothiazol-2-ylamino)-1-methyl-1H-benzoimidazole-5- |
| | carboxylic acid [2-(4-dimethylsulfamoyl-piperazin-1-yl)-2-oxo-ethyl]-amide |
| 431 | 1-Methyl-2-(6-trifluoromethoxy-benzothiazol-2-ylamino)-1H- |
| | benzoimidazole-5-carboxylic acid [2-(3-hydroxy-pipendin-1-yl)-2-oxo- |
| | ethyl]-amide |
| 432 | 1-Methyl-2-(6-trifluoromethyl-benzothiazol-2-ylamino)-1H- |
| | benzoimidazole-5-carboxylic acid [2-((R)-3-hydroxy-piperidin-1-yl)-2-oxo- |
| | ethyl]-amide |
| 433 | 2-(6-Chloro-benzothiazol-2-ylamino)-1-methyl-1H-benzoimidazole-5- |
| | carboxylic acid [2-((S)-3-hydroxy-piperidin-1-yl)-2-oxo-ethyl]-amide |
| 434 | 1-Methyl-2-(6-trifluoromethyl-benzothiazol-2-ylamino)-1H- |
| | benzoimidazole-5-carboxylic acid [2-((S)-3-hydroxy-piperidin-1-yl)-2-oxo- |
| | ethyl]-amide |
| 435 | 1-Methyl-2-(6-trifluoromethoxy-benzothiazol-2-ylamino)-1H- |
| | benzoimidazole-5-carboxylic acid [2-(4-hydroxy-pipendin-1-yl)-2-oxo- |
| | ethyl]-amide |
| 436 | 2-(6-Chloro-benzothiazol-2-ylamino)-1-methyl-1H-benzoimidazole-5- |
| | carboxylic acid [2-(4-hydroxy-pipendin-1-yl)-2-oxo-ethyl]-amide |
| 437 | 1-Methyl-2-(6-trifluoromethyl-benzothiazol-2-ylamino)-1H- |
| | benzoimidazole-5-carboxylic acid [2-(4-hydroxy-pipendin-1-yl)-2-oxo- |
| | ethyl]-amide |
| 438 | 2-(6-Chloro-benzothiazol-2-ylamino)-1-(2-methoxy-ethyl)-1H- |
| | benzoimidazole-5-carboxylic acid [2-(4-hydroxy-pipendin-1-yl)-2-oxo- |
| | ethyl]-amide |
| 439 | 1-Methyl-2-(6-trifluoromethoxy-benzothiazol-2-ylamino)-1H- |
| | benzoimidazole-5-carboxylic acid [2-(2-hydroxymethyl-pipendin-1-yl)-2- |
| | oxo-ethyl]-amide |
| 440 | 1-Methyl-2-(6-trifluoromethoxy-benzothiazol-2-ylamino)-1H- |
| | benzoimidazole-5-carboxylic acid [2-(3-hydroxymethyl-pipendin-1-yl)-2- |
| | oxo-ethyl]-amide |
| 441 | 1-Methyl-2-(6-trifluoromethoxy-benzothiazol-2-ylamino)-1H- |
| | benzoimidazole-5-carboxylic acid [2-(4-hydroxymethyl-pipendin-1-yl)-2- |
| | oxo-ethyl]-amide |
| 442 | 2-(6-Chloro-benzothiazol-2-ylamino)-1-methyl-1H-benzoimidazole-5- |
| | carboxylic acid [2-(4-hydroxymethyl-piperidin-1-yl)-2-oxo-ethyl]-amide |
| 443 | 2-(6-Chloro-benzothiazol-2-ylamino)-1-(2-methoxy-ethyl)-1H- |
| | benzoimidazole-5-carboxylic acid [2-(4-hydroxymethyl-pipendin-1-yl)-2- |
| | oxo-ethyl]-amide |
| 444 | 2-(6-Chloro-benzothiazol-2-ylamino)-1-methyl-1H-benzoimidazole-5- |
| | carboxylic acid [2-((S)-3-methoxy-piperidin-1-yl)-2-oxo-ethyl]-amide |
| 445 | 1-Methyl-2-(6-trifluoromethyl-benzothiazol-2-ylamino)-1H- |
| | benzoimidazole-5-carboxylic acid [2-((S)-3-methoxy-piperidin-1-yl)-2-oxo- |
| | ethyl]-amide |
| 446 | 1-Methyl-2-(6-trifluoromethyl-benzothiazol-2-ylamino)-1H- |
| | benzoimidazole-5-carboxylic acid [2-((R)-3-methoxy-piperidin-1-yl)-2-oxo- |
| | ethyl]-amide |
| 447 | 1-Methyl-2-(6-trifluoromethoxy-benzothiazol-2-ylamino)-1H- |
| | benzoimidazole-5-carboxylic acid [2-(4-methoxymethyl-piperidin-1-yl)-2- |
| | oxo-ethyl]-amide |
| 448 | 1-Methyl-2-(6-trifluoromethoxy-benzothiazol-2-ylamino)-1H- |
| | benzoimidazole-5-carboxylic acid [2-(4-fluoromethyl-piperidin-1-yl)-2-oxo- |
| | ethyl]-amide |
| 449 | 1-Methyl-2-(6-trifluoromethoxy-benzothiazol-2-ylamino)-1H- |
| | benzoimidazole-5-carboxylic acid [2-oxo-2-(4-trifluoromethyl-piperidin-1- |
| | yl)-ethyl]-amide |
| 450 | 1-Methyl-2-(6-trifluoromethoxy-benzothiazol-2-ylamino)-1H- |
| | benzoimidazole-5-carboxylic acid [2-(4-cyano-piperidin-1-yl)-2-oxo-ethyl]- |
| | amide |
| 451 | 1-Methyl-2-(6-trifluoromethoxy-benzothiazol-2-ylamino)-1H- |
| | benzoimidazole-5-carboxylic acid [2-(4-carbamoyl-piperidin-1-yl)-2-oxo- |
| | ethyl]-amide |
| 452 | 1-Methyl-2-(6-trifluoromethoxy-benzothiazol-2-ylamino)-1H- |
| | benzoimidazole-5-carboxylic acid {2-oxo-2-[4-(pyrimidin-2-yloxy)- |
| | piperidin-1-yl]-ethyl}-amide |
| 453 | 1-Methyl-2-(6-trifluoromethoxy-benzothiazol-2-ylamino)-1H- |
| | benzoimidazole-5-carboxylic acid [2-(4-methylamino-piperidin-1-yl)-2-oxo- |
| | ethyl]-amide hydrochloride |
| 454 | 2-(6-Chloro-benzothiazol-2-ylamino-1-methyl-1H-benzoimidazole-5- |
| | carboxylic acid [2-(4-methylamino-piperidin-1-yl)-2-oxo-ethyl]-amide |
| | hydrochloride |
| 455 | 1-Methyl-2-(6-trifluoromethoxy-benzothiazol-2-ylamino)-1H- |
| | benzoimidazole-5-carboxylic acid [2-(4-dimethylamino-piperidin-1-yl)-2- |
| | oxo-ethyl]-amide |
| 456 | 2-(6-Chloro-benzothiazol-2-ylamino)-1-methyl-1H-benzoimidazole-5- |
| | carboxylic acid [2-(4-dimethylamino-piperidin-1-yl)-2-oxo-ethyl]-amide |
| 457 | 2-(6-Chloro-benzothiazol-2-ylamino)-1-methyl-1H-benzoimidazole-5- |
| | carboxylic acid [2-(4-methylaminomethyl-piperidin-1-yl)-2-oxo-ethyl]- |
| | amide hydrochloride |
| 458 | 1-Methyl-2-(6-trifluoromethoxy-benzothiazol-2-ylamino)-1H- |
| | benzoimidazole-5-carboxylic acid [2-(4-dimethylaminomethyl-piperidin-1- |
| | yl)-2-oxo-ethyl]-amide |
| 459 | 2-(6-Chloro-benzothiazol-2-ylamino)-1-methyl-1H-benzoimidazole-5- |
| | carboxylic acid [2-(4-dimethylaminomethyl-piperidin-1-yl)-2-oxo-ethyl]- |
| | amide |
| 460 | 2-(6-Chloro-benzothiazol-2-ylamino)-1-methyl-1H-benzoimidazole-5- |
| | carboxylic acid [2-(3-methylaminomethyl-piperidin-1-yl)-2-oxo-ethyl]-amid |
| | hydrochloride |
| 461 | 2-(6-Chloro-benzothiazol-2-ylamino)-1-methyl-1H-benzoimidazole-5- |
| | carboxylic acid [2-(3-dimethylaminomethyl-piperidin-1-yl)-2-oxo-ethyl]- |
| | amide |
| 462 | 2-(6-Chloro-benzothiazol-2-ylamino)-1-methyl-1H-benzoimidazole-5- |
| | carboxylic acid ((S)-1-dimethylcarbamoyl-ethyl)-amide |
| 463 | 1-Methyl-2-(6-trifluoromethyl-benzothiazol-2-ylamino)-1H- |
| | benzoimidazole-5-carboxylic acid ((S)-1-dimethylcarbamoyl-ethyl)-amide |
| 464 | 2-(6-Chloro-benzothiazol-2-ylamino)-1-methyl-1H-benzoimidazole-5- |
| | carboxylic acid [(S)-1-methyl-2-(4-methyl-piperazin-1-yl)-2-oxo-ethyl]- |
| | amide |
| 465 | 2-(6-Chloro-benzothiazol-2-ylamino)-1-methyl-1H-benzoimidazole-5- |
| | carboxylic acid ((S)-1-methyl-2-morpholin-4-yl-2-oxo-ethyl)-amide |
| 466 | 1-Methyl-2-(6-trifluoromethyl-benzothiazol-2-ylamino)-1H- |
| | benzoimidazole-5-carboxylic acid ((S)-1-methyl-2-morpholin-4-yl-2-oxo- |
| | ethyl)-amide |
| 467 | 2-(6-Chloro-benzothiazol-2-ylamino)-1-methyl-1H-benzoimidazole-5- |
| | carboxylic acid ((R)-1-dimethylcarbamoyl-ethyl)-amide |
| 468 | 1-Methyl-2-(6-trifluoromethyl-benzothiazol-2-ylamino)-1H- |
| | benzoimidazole-5-carboxylic acid ((R)-1-dimethylcarbamoyl-ethyl)-amide |
| 469 | 2-(6-Chloro-benzothiazol-2-ylamino)-1-methyl-1H-benzoimidazole-5- |
| | carboxylic acid [(R)-1-methyl-2-(4-methyl-piperazin-1-yl)-2-oxo-ethyl]- |
| | amide |
| 470 | 1-Methyl-2-(6-trifluoromethyl-benzothiazol-2-ylamino)-1H- |
| | benzoimidazole-5-carboxylic acid [(R)-1-methyl-2-(4-methyl-piperazin-1- |
| | yl)-2-oxo-ethyl]-amide |
| 471 | 2-(6-Chloro-benzothiazol-2-ylamino)-1-methyl-1H-benzoimidazole-5- |
| | carboxylic acid ((R)-1-methyl-2-morpholin-4-yl-2-oxo-ethyl)-amide |
| 472 | 1-Methyl-2-(6-trifluoromethyl-benzothiazol-2-ylamino)-1H- |
| | benzoimidazole-5-carboxylic acid ((R)-1-methyl-2-morpholin-4-yl-2-oxo- |
| | ethyl)-amide |
| 473 | N-[2-(2-Hydroxyethoxy)ethyl]-3-methyl-2-[[6-(trifluoromethyl)-1,3- |
| | benzothiazol-2-yl]amino]imidazo[4,5-b]pyridine-6-carboxamide |
| 474 | 1-Ethyl-N-[2-(2-hydroxyethoxy)ethyl]-2-[[5-(trifluoromethoxy)-1,3- |
| | benzothiazol-2-yl]amino]benzimidazole-5-carboxamide |

Compounds 1-474 in Table A may be prepared as described in WO '018 or other methods apparent to one of skill in the art. For example, Compounds 473 and 474 in Table A may be prepared as described in the Examples section below.

The compounds of the present invention can be used in the form of pharmaceutically acceptable salts derived from inorganic or organic acids or bases. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, Vol 66(1), pp. 1-19 (1977).

The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, hyaluronic acid, malic acid, sulphuric acid and phosphoric acid and such organic acids as oxalic acid, malic acid, maleic acid, methanosulfonic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

In another aspect, the present invention provides a pharmaceutical composition comprising the compound of Formula (I) or a pharmaceutically acceptable salt thereof for use in manufacture of a medicine or for use in treating a refractive ocular condition. In an embodiment, the present invention provides a pharmaceutical composition comprising a compound (or salt) of any one of embodiments 1 to 250 (recited above) and a pharmaceutical carrier. In another embodiment, the pharmaceutical composition comprises a compound (or salt) of any one of the examples 1-474 and a pharmaceutically acceptable carrier.

Thus, in another embodiment, the invention provides a pharmaceutical composition comprising a compound of Formula (I) or a pharmaceutically acceptable salt thereof, and a pharmaceutically acceptable carrier. In another embodiment, the invention provides a pharmaceutical composition comprising a compound (or salt) of any one of embodiments 1 to 250 or a compound (or salt) of any one of Examples 1-474 and a pharmaceutical acceptable carrier.

In another embodiment, the present invention provides a compound of Formula (I) or a pharmaceutically acceptable salt thereof for use in medicine. In another embodiment, the invention provides a compound (or salt) of any one of embodiments 1 to 250 or a compound (or salt) of any one of Examples 1-474 for use in medicine.

B. Co-Administration

The present invention further provides for the use of a compound of Formula (I) or a pharmaceutically acceptable salt thereof, in combination with one or more active compounds for simultaneous, subsequent, or sequential administration. The invention also provides for the use of a compound (or salt) of any one of embodiments 1 to 250 or a compound (or salt) of any one of Examples 1-474 in combination with one or more active compounds for simultaneous, subsequent, or sequential administration.

In one embodiment, the invention provides a pharmaceutical composition comprising a compound (or salt) of any one of embodiments 1 to 250 or a compound (or salt) of any one of Examples 1-474 and at least one other active ingredient selected from lipoic acid, lipoic acid derivatives (such as lipoic acid choline ester), Nrf2 activators, antioxidants, detoxification agents, and anti-inflammatory agents.

In another embodiment, the additional active agent is selected from the group consisting of general miotic agents, such as a non-selective muscarinic acetylcholine receptor agonist (pilocarpine), a non-selective muscarinic acetylcholine receptor agonist (carbachol), an acetylcholinesterase inhibitor (phospholine iodide), or an alpha 2 selective vasoconstrictor (brimonidine).

In another embodiment, the invention provides for the use of a compound (or salt) of any one of embodiments 1 to 250 or a compound (or salt) of any one of Examples 1-474 in combination with at least one other medically effective active ingredient selected from lipoic acid, lipoic acid derivatives (such as lipoic acid choline ester), Nrf2 activators, antioxidants, detoxification agents, and anti-inflammatory agents for simultaneous, subsequent, or sequential administration. In another embodiment, the additional active agent is selected from the group consisting of general miotic agents, such as a non-selective muscarinic acetylcholine receptor agonist (pilocarpine), a non-selective muscarinic acetylcholine receptor agonist (carbachol), an acetylcholinesterase inhibitor (phospholine iodide), or an alpha 2 selective vasoconstrictor (brimonidine).

In some embodiments, the compositions disclosed herein are co-administered in combination with one or more additional active agent for treatment of a refractive ocular condition.

C. Dosages and Dosage Regimes

The selected dosage depends upon the desired therapeutic effect, on the route of administration, and on the duration of the treatment desired. Dosage amounts of 0.001 to 100 mg/kg of body weight daily may be administered to a subject.

An appropriate dose of a compound of Formula (I) or a pharmaceutically acceptable salt thereof for use in the present invention, may be determined according to any one of several well-established protocols. For example, animal studies such as studies using mice, rats, dogs, and/or monkeys may be used to determine an appropriate dose of a pharmaceutical compound. Results from animal studies may be extrapolated to determine doses for use in other species, such as for example, humans.

A compound of Formula (I) or a pharmaceutically acceptable salt thereof may be administered in a dosage of between 0.01 mg and 15 mg per kg. In an embodiment, dose may be between 0.01 mg and 1000 mg. In another embodiment, a compound of Formula (I) or a pharmaceutically acceptable salt thereof is administered orally in an amount from 10 mg/day to 1000 mg/day, or from 25 mg/day to 800 mg/day, or from 37 mg/day to 750 mg/day, or from 75 mg/day to 700 mg/day, or from 100 mg/day to 600 mg/day, or from 150 mg/day to 500 mg/day, or from 200 mg/day to 400 mg/day. In another embodiment, a compound of Formula (I) or a pharmaceutically acceptable salt thereof is administered in ocularly in an amount from 0.010 mg/day to 5 mg/day, or from 0.025 mg/day to 1 mg/day, or from 0.050 mg/day to 1 mg/day, or from 0.100 mg/day to 1 mg/day, or from 0.250 mg/day to 1 mg/day, or from 0.50 mg/day to 1 mg/day. In other embodiments, the previous daily periods of administration of an amount of a compound of Formula (I) or a pharmaceutically acceptable salt thereof may be changed to a period of every 6 hours (or 4 times a day), 8 hours (or 3 times a day), 12 hours (or twice a day), 48 hours, 72 hours, 96 hours, 1 week, or 2 weeks. In some embodiments, the compound or composition is administered to the subject four times per day (QID), three times per day (TID), two times per day (BID), or once per day (QD).

In some embodiments, the composition is administered as part of a dosing regimen. For example, the patient can be administered a first dose of the composition for a first dosing period; and a second dose of the composition for a second dosing period, optionally followed by one or more additional doses for one or more additional dosing periods. The first dosing period can be less than one week, one week, or more than one week.

In some embodiments, the dosage regime is a dose escalating dosage regime.

D. Formulations

Pharmaceutical compositions comprising a compound of the invention are disclosed. The pharmaceutical compositions may be for administration by oral, parenteral (intramuscular, intraperitoneal, intravenous (IV) or subcutaneous injection), transdermal (either passively or using iontophoresis or electroporation), transmucosal (nasal, vaginal, rectal, or sublingual), or ocular routes of administration and can be formulated in unit dosage forms appropriate for each route of administration.

In some embodiments, a compound of the invention or a pharmaceutical composition is administered locally, to the site in need of therapy. For example, in some embodiments, a compound of the invention or a pharmaceutical composition is administered locally to the eye.

In an embodiment, the pharmaceutical compositions are formulated for oral delivery. Oral solid dosage forms are described generally in Remington's Pharmaceutical Sciences, 21th Ed. 2005 at Chapter 45. Solid dosage forms include tablets, capsules, pills, troches or lozenges, cachets, pellets, powders, or granules or incorporation of the material into particulate preparations of polymeric compounds such as polylactic acid, polyglycolic acid, etc., or into liposomes. Such compositions may influence the physical state, stability, rate of in vivo release, and rate of in vivo clearance of the disclosed. The compositions may be prepared in liquid form, or may be in dried powder (e.g., lyophilized) form. Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. Compounds of the invention can be incorporated into an inert matrix which permits release by either diffusion or leaching mechanisms, e.g., gums. Slowly degenerating matrices may also be incorporated into the formulation.

The amount of the active agent (e.g., compound of Formula (I) or a pharmaceutically acceptable salt thereof) in an ocular pharmaceutical formulation can be selected based on the condition of the subject to be treated, including the subject's age, gender, as well as vision and lens status.

Ocular formulations include, but are not limited to, liquid formulations (e.g., solutions, suspensions) for topical administration as well as formulation for injection or ocular insert administration. The ocular formulation may be formulated for topical administration such as an eye drop, swab, ointment, gel, or mist (e.g, an aerosol or spray). In one embodiment, the formulation is an eye drop. For ocular formulations, the pharmaceutically acceptable excipients are selected to be compatible with, and suitable for, ocular use. Such excipients are well known in the art. In one embodiment, excipients may be selected to improve the solubility of the agent.

Exemplary amounts of a compound of Formula (I) or a pharmaceutically acceptable salt thereof in an ocular formulation can be about 0.01% to about 20% w/v, about 0.1% to about 7.5% w/v, about 0.15% to about 5.0% w/v, about 0.5% to about 4.0% w/v, about 1.0% to about 4.0% w/v, about 3.0% w/v, or about 2% w/v. In another embodiment, the amount of a compound of Formula (I) or a pharmaceutically acceptable salt thereof can be about 0.01% to about 10% w/v, about 0.1% to about 7.5% w/v, about 0.15% to about 5.0% w/v, about 0.5% to about 4.0% w/v, about 1.0% to about 4.0% w/v, about 3.0% w/v, or about 2% w/v.

Excipients for an ocular formulation may include, but are not limited to, buffers, tonicity agents, viscosity agents, preservatives, preservatives, surfactants, emulsifiers, salts, lubricants, polymers, solvents, and other known excipients for ocular pharmaceutical formulations. Appropriate amounts can be determined by one of ordinary skill in the art, but non-limiting exemplary amounts (in % by weight) are also provided below.

In one embodiment, the ocular pharmaceutical composition may include one or more buffers to adjust or maintain the pH of the formulation. In one embodiment, the pH is near physiological pH of about 7. Thus, the pH of the formulation can be between about 4 to about 8, or about 5 to about 8, or about 6 to about 7, or about 6.8 to about 7.2, or about 7.1 to about 7.5. In another embodiment, the pH is about 5.5, or about 5 to about 7, or about 4.5 to about 6, or about 4.5 to about 5.5, or about 5.5 to about 6.5, or about 5 to about 6, or about 5.25 to about 5.75, or about 5.5. Exemplary buffers include, but are not limited to, phosphate buffers (e.g., sodium phosphate monobasic monohydrate, sodium phosphate dibasic anhydrous), borate buffers, and HBSS (Hank's Balanced Salt Solution). In one embodiment, the buffer is a phosphate buffer. In another embodiment, the buffer is sodium phosphate monobasic monohydrate and/or sodium phosphate dibasic anhydrous. The buffer amount (amount of either total buffer or a single buffer excipient) can be 0.1% to about 3.0% w/v, about 0.2% to about 2.0% w/v, about 0.5% to about 2.5% w/v, about 0.75% to about 2.25% w/v, about 1.5% to about 2.5% w/v, or about 1.0% to about 2.0% w/v.

In one embodiment, the ocular pharmaceutical composition may include one or more tonicity agents. Although the formulation may be hypertonic or hypotonic, isotonic formulations are preferred (260-320 mOsm). Exemplary tonicity agents include, but are not limited to, sodium chloride. The tonicity agent amount can be about 0.1% to about 5% w/v, about 0.1% to about 2% w/v, about 0.1% to about 1% w/v, about 0.25% to about 0.75% w/v, about 0.2% to about 0.6% w/v, or about 0.5% w/v.

In one embodiment the ocular pharmaceutical composition may include one or more viscosity agents to increase the viscosity of the formulation. Exemplary viscosity agents include, but are not limited to, cellulosic agents (e.g., hydroxypropyl methylcellulose), polycarbophil, polyvinyl alcohol. In one embodiment, the viscosity agent is a cellulosic agent, e.g., hydroxypropyl methylcellulose. The viscosity agent amount can be about 0.1% to about 5% w/v, about 0.1% to about 2% w/v, about 0.1% to about 1% w/v, about 0.1% to about 0.4% w/v, or about 0.2% w/v.

In one embodiment, the ocular pharmaceutical composition may include one or more preservatives to minimize microbial contamination or enhance shelf life. Exemplary preservatives include, but are not limited to, benzalkonium chloride (BAK), cetrimonium, chlorobutanol, edetate disodium (EDTA), polyquaternium-1 (Polyquad™), polyhexamethylene biguanide (PHMB), stabilized oxychloro complex (PURITE™), sodium perborate, and Soflia™. The preservative amount may be, e.g., less than about 0.02% w/v, about 0.004% w/v or less, or about 0.005% to about 0.01% w/v.

In one embodiment, the ocular pharmaceutical composition may include one or more stabilizers. Exemplary stabilizers include, but are not limited to, amino acids such as alanine. The stabilizer amount can be about 0.1% to about 5% w/v, about 0.1% to about 2% w/v, about 0.1% to about 1% w/v, about 0.25% to about 0.75% w/v, about 0.2% to about 0.6% w/v, or about 0.5% w/v.

In one embodiment, the ocular pharmaceutical composition may include one or more surfactants. Exemplary surfactants include, but are not limited to, cyclodextrins, polyoxyl alkyls, poloxamers or combinations thereof, and may include in addition combinations with other surfactants such as polysorbates. Embodiments include polyoxyl 40 stearate and optionally Poloxamer 108, Poloxamer 188, Poloxamer 407, Polysorbate 20, Polysorbate 80, ionically charged (e.g. anionic) beta-cyclodextrins with or without a butyrated salt (Captisol™) 2-hydroxypropyl beta cyclodextrin (“HPBCD”), alpha cyclodextrins, gamma cyclodextrins, Polyoxyl 35 castor oil, and Polyoxyl 40 hydrogenated castor oil or combinations thereof. In certain embodiments, the addition of an anionic surfactant such as sodium lauryl sulfate and or sodium ester lauryl sulfate may be preferred. In other embodiments, the addition of polysorbate 80 may be preferred. The amount surfactant (either total surfactant or a single surfactant) can be 0.1% to about 3.0% w/v, about 0.2% to about 2.0% w/v, about 0.5% to about 2.5% w/v, about 0.75% to about 2.25% w/v, about 1.5% to about 2.5% w/v, or about 1.0% to about 2.0% w/v.

### Examples

### General Procedure A: Ipso substitution of 6-chloro-5-nitro-nicotinic acid methyl ester

To a DMF or THF solution of a 6-chloro-5-nitro-nicotinic acid methyl ester is added 2 M methylamine in THF and the reaction mixture stirred at room temperature for 16 h. The resulting mixture is poured into water to precipitate the product. The precipitate may be filtered and dried to give the product, which may not be purified further before use in the next step.

### General Procedure B: Reduction of Nitro Group to Amine

10% Pd/C is added to a solution of the nitro compound in methanol. The resulting mixture is stirred at room temperature under a 112 atmosphere for 16 h. The contents may then be filtered through a pad of Celite or silica gel and the solid washed with portions of methanol. The filtrate and washings are combined and evaporated to afford the corresponding diamine, which may not be purified further before use in the next step.

### General Procedure C: Thiourea formation and its conversion to 2-amino-imidazopyridine

1,1′-Thiocarbonylimidazole is added to a solution of an amine with triethylamine (1 eq.) in acetonitrile (10 mL). The reaction mixture is stirred at room temperature (1-24 h). The solvent is then evaporated, and the product suspended in acetonitrile. The solvent is then evaporated to produce the product as a precipitate. The precipitate is filtered and washed with acetonitrile and dried. The product may be used directly in the next step without further purification.

To the product obtained immediately above is added EDAC at room temperature followed by a substituted diaminopyridine, and the reaction mixture is stirred at 90° C. for 16 h. The reaction mixture is then cooled to room temperature, poured into cold water, and the solid collected by filtration. The crude product thus obtained may be purified by trituration with methanol.

### General Procedure D: Hydrolysis of Ester

A solution of NaOH in water is added to a solution of an ester in 1:1 THF/MeOH, and the resulting mixture is stirred at 60° C. for 16 h. After completion of the reaction, the mixture is concentrated under vacuum. The pH of the resulting suspension may be adjusted by the dropwise addition of 6 N HCl to pH ˜3, and the precipitate collected by filtration, washed with water and dried under vacuum. The desired carboxylic acid may be used without purification.

### General Procedure E: Amide Formation Using HBTU as Coupling Reagent

To a solution of a carboxylic acid in dry DMF is added DIEA followed by HBTU, and the reaction mixture is stirred at room temperature for 30 min. An appropriate amine is then added, and the reaction stirred at room temperature for 16 h. The contents may be diluted with ice-water, and the product precipitated. The product may be isolated after filtration either with subsequent washings with water and DCM/methanol or through silica gel chromatography using hexanes/ethyl acetate (from 80:20 to 60:40) as an eluent system.

Compound 473

### N-[2-(2-Hydroxyethoxy)ethyl]-3-methyl-2-[[6-(trifluoromethyl)-1,3-benzothiazol-2-yl]amino]imidazo[4,5-b]pyridine-6-carboxamide

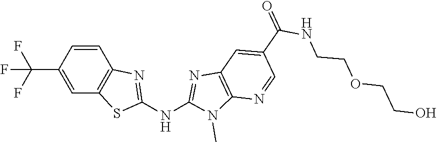 [SMILES: Cn1c(Nc2nc3ccc(C(F)(F)F)cc3s2)nc2cc(C(=O)NCCOCCO)cnc21]

6-Methylamino-5-nitro-nicotinic acid methyl ester (5.0 g) was prepared by following General Procedure A starting from 6-chloro-5-nitro-nicotinic acid methyl ester (5.0 g) and methylamine (33% in EtOH, 24 mL) in THE (150 mL). The crude product was used in the next step without further purification.

5-Amino-6-methylamino-nicotinic acid methyl ester (4.8 g) was prepared by following General Procedure B starting from 6-methylamino-5-nitro-nicotinic acid methyl ester (5.0 g) and Pd/C (20% by weight, 1.0 g) in methanol:THF (1:1, 50 mL). The crude product was used in the next step without further purification.

Methyl 3-methyl-2-[[6-(trifluoromethyl)-1,3-benzothiazol-2-yl]amino]imidazo[4,5-b]pyridine-6-carboxylate (5.0 g) was prepared by following General Procedure C starting from 6-(trifluoromethyl)-1,3-benzothiazol-2-amine (5.0 g), 5-amino-6-methylamino-nicotinic acid methyl ester (5.0 g), 1,1′-thiocarbonyl-diimidazole (5.0 g), and EDAC (4.5 g). The crude product was used in next step without further purification.

3-Methyl-2-[[6-(trifluoromethyl)-1,3-benzothiazol-2-yl]amino]imidazo[4,5-b]pyridine-6-carboxylic acid (4.2 g) was prepared by following General Procedure D starting from methyl 3-methyl-2-[[6-(trifluoromethyl)-1,3-benzothiazol-2-yl]amino]imidazo[4,5-b]pyridine-6-carboxylate (5.0 g) and NaOH (2N, 25 mL) in methanol:THF (2:1, 50 mL). The crude product was used in next step without further purification.

N-[2-(2-Hydroxyethoxy)ethyl]-3-methyl-2-[[6-(trifluoromethyl)-1,3-benzothiazol-2-yl]amino]imidazo[4,5-b]pyridine-6-carboxamide (40 mg) was prepared by following General Procedure E starting from 3-methyl-2-[[6-(trifluoromethyl)-1,3-benzothiazol-2-yl]amino]imidazo[4,5-b]pyridine-6-carboxylic acid (100 mg), 2-(2-aminoethoxy)ethanol (100 mg), HBTU (200 mg) and DIEA (0.2 mL) in DMF (2.0 mL). LC/MS: m/z 481.7. ¹H NMR (DMSO-ds, 400 MHz): δ 8.67-8.59 (m, 211), 8.29-8.23 (d, 211), 7.71-7.69 (d, 1H), 4.61 (s, 1H), 3.68 (br s, 3H), 3.57-3.44 (m, 811), 3.32 (br s, 2H).

Compound 474

### 1-Ethyl-N-[2-(2-hydroxyethoxy)ethyl]-2-[[5-(trifluoromethoxy)-1,3-benzothiazol-2-yl]amino]benzimidazole-5-carboxamide

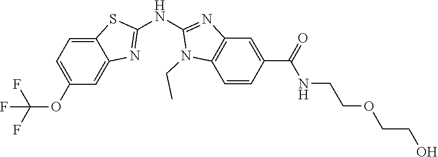 [SMILES: CCn1c(Nc2nc3cc(OC(F)(F)F)ccc3s2)nc2cc(C(=O)NCCOCCO)ccc21]

1-Ethyl-N-[2-(2-hydroxyethoxy)ethyl]-2-[[5-(trifluoromethoxy)-1,3-benzothiazol-2-yl]amino]benzimidazole-5-carboxamide (40 mg) was prepared by following General Procedure E starting from 3-ethyl-2-[[6-(trifluoromethoxy)-1,3-benzothiazol-2-yl]amino]imidazo[4,5-b]pyridine-6-carboxylic acid (100 mg) (See WO 018), 2-(2-aminoethoxy)ethanol (100 mg), HBTU (200 mg) and DIEA (0.2 mL) in DMF (2.0 mL). LC/MS: m/z 510.7.

### Example 1—In Vitro Pharmacodynamic Study on Lens Elasticity

Purpose

Evaluate effect of test compounds (Examples 46 and 134) on the changes in lens elasticity in vitro in the lens from aged mice.

Methods

Test compounds (Examples 46 and 134) at 50 μM and 500 μM, Positive Control of Lipoic Acid at 50 μM and 500 μM, and control treatment were administered to lenses (12 per group) according to methods described in Invest Ophthalmol Vis Sci. 2016; 57:2851-2863. Briefly, seven month old C57BL/6J mice (male) were euthanized by CO₂ overdose. Lenses were removed and placed in culture medium. Lenses were incubated for about 2 hours at 37° C. The medium was replaced with fresh medium supplemented with test compounds or with fresh medium. After about 14 hours incubation at 37° C., lenses were removed from culture medium.

Measurement of lens elasticity was performed according to the methods described in Invest Ophthalmol Vis Sci. 2016; 57:2851-2863. Briefly, lenses were placed on slides and photographed. A second photograph was taken after a coverslip was placed on the lens. Equatorial diameters changed by the coverslips placed on the lens were analyzed.

Results

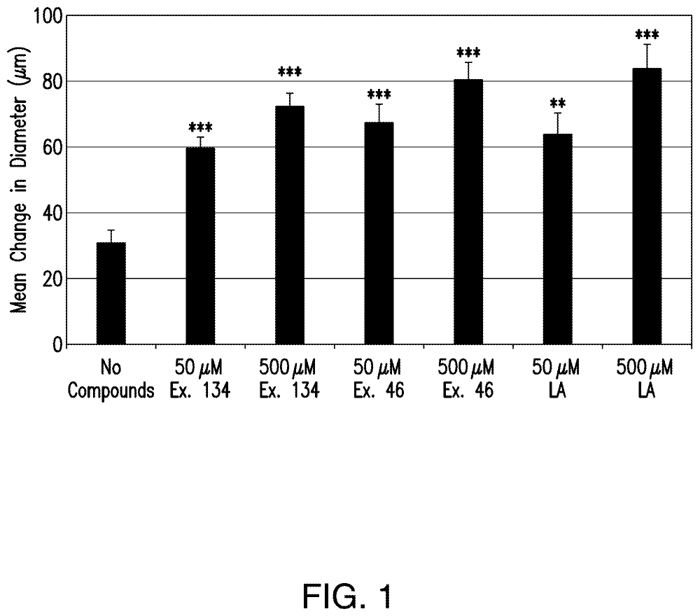

Treatment of lenses with Examples 46 and 134 at both concentrations of 50 μM and 500 μM significantly increased lens elasticity relative to control. Further, there was dose dependent increase in elasticity with Examples 46 and 134 from the lower dose (50 μM) to the higher dose (500 μM). Further, the efficacy of Examples 46 and 134 were almost the same as that of the positive control groups using lipoic acid. A summary of the results is provided in Table 1 and in FIG. 1.

TABLE 1
Changes in diameter of mouse lens

| | | Change in Diameter (μm) |
|---|---|---|
| Group | Test Article | (Mean ± S.E.) |
| 1 | Medium (no compound) | 30.8 ± 3.9 |
| 2 | Ex. 134 (50 μM) | 59.6 ± 3.4 |
| 3 | Ex. 134 (500 μM) | 72.3 ± 4.0 |
| 4 | Ex. 46 (50 μM) | 67.4 ± 5.6 |
| 5 | Ex. 46 (500 μM) | 80.4 ± 5.3 |
| 6 | Lipoic Acid (50 μM) | 63.8 ± 6.5 |
| 7 | Lipoic Acid (500 μM) | 83.8 ± 7.4 |

### Example 2—In Vitro Pharmacodynamic Study on Lens Elasticity Under 2-Week Repeated Instillation

Purpose

Evaluate effect of topically applied test compounds (Examples 46 and 134) on the changes of lens elasticity in aged mice.

Methods

Test compounds (Examples 46 and 134), positive control of Lipoic Acid Choline Ester (LACE), and control treatment were topically administered to right eyes according to methods described in Invest Ophthalmol Vis Sci. 2016; 57:2851-2863. Briefly, eight month old C57BL/6J mice (male) were treated with 2.5 μL of each test sample three times per day (TID) at about 4-hour intervals in the right eyes for 2 weeks. The left eyes were used as untreated controls. Each treatment group included 10 mice, and the vehicle group included 5 mice.

Measurement of lens elasticity was performed according to the methods described in Invest Ophthalmol Vis Sci. 2016; 57:2851-2863. Briefly, after 2-week treatment, mice were euthanized by CO₂ overdose. Lenses were removed and analyzed for changes in elasticity. Lenses were placed on slides and photographed. A second photograph was taken after a coverslip was placed on the lens. Equatorial diameters changed by the coverslips placed on the lens were analyzed.

Results

Repeated installation of 0.15%, 0.5%, 1.5% solutions comprising the compound of Example 46 or Example 134 TID significantly increased elasticity of the mouse lenses. The efficacy of 0.5% and 1.5% solutions comprising the compound of Example 46 or Example 134 were better than or equivalent to that of 1.5% solutions comprising LACE.

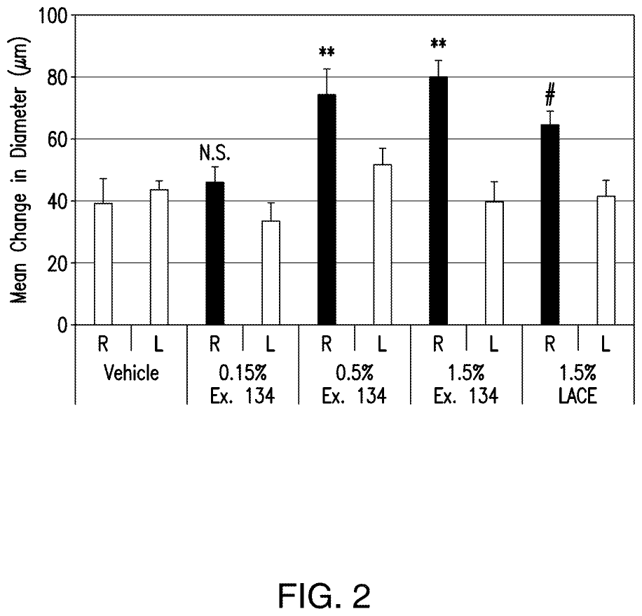

A summary of the results for the compound of Example 134 is provided in Table 2 and in FIG. 2.

TABLE 2
Changes in diameter of mouse lens

| | | Right Eye Change | Left Eye Change |
|---|---|---|---|
| | Test Article | in Diameter (μm) | in Diameter (μm) |
| Group | (Right Eye) | (Mean ± S.E.) | (Mean ± S.E.) |
| 1 | Vehicle | 39.2 ± 8.0 | 43.6 ± 2.9 |
| 2 | 0.15% Ex. 134 | 46.0 ± 5.0 | 33.5 ± 5.9 |
| 3 | 0.5% Ex. 134 | 74.3 ± 8.3 | 51.7 ± 5.3 |
| 4 | 1.5% Ex. 134 | 80.0 ± 5.3 | 39.7 ± 6.5 |
| 5 | 1.5% LACE | 64.5 ± 4.5 | 41.5 ± 5.2 |

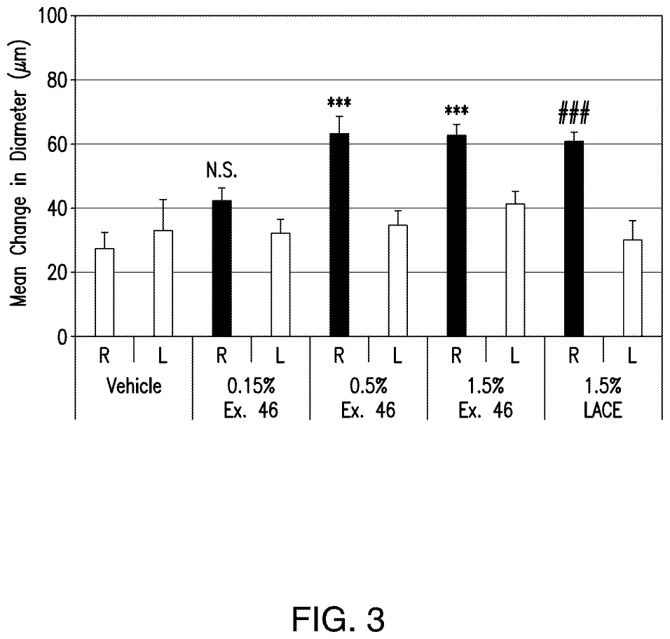

A summary of the results for the compound of Example 134 is provided in Table 3 and in FIG. 3.

TABLE 3
Changes in diameter of mouse lens

| | | Right Eye Change | Left Eye Change |
|---|---|---|---|
| | Test Article | in Diameter (μm) | in Diameter (μm) |
| Group | (Right Eye) | (Mean ± S.E.) | (Mean ± S.E.) |
| 1 | Vehicle | 27.4 ± 5.0 | 33.0 ± 9.7 |
| 2 | 0.15% Ex. 46 | 42.3 ± 4.0 | 32.2 ± 4.3 |
| 3 | 0.5% Ex. 46 | 63.2 ± 5.4 | 34.7 ± 4.5 |
| 4 | 1.5% Ex. 46 | 62.7 ± 3.4 | 41.3 ± 3.9 |
| 5 | 1.5% LACE | 60.8 ± 2.9 | 30.1 ± 6.0 |

What is claimed is:

1. A method of treating a refractive ocular disorder in a subject comprising:
administering to the subject a therapeutically effective amount of a compound of Formula (I) or a pharmaceutically acceptable salt thereof,
wherein a compound of Formula (I) has the structure shown below 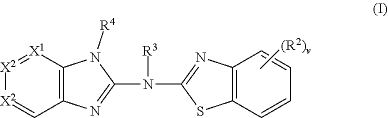
wherein
X¹ is ═N— or ═CH—;
X² is ═C(R¹)— and X³ is ═C(-L-G)-; or X² is ═C(-L-G)- and X³ is ═C(R¹)—;
G is hydrogen, —C₁₋₈ alkyl, —C₃₋₁₀ cycloalkyl, —C₁₋₆ alkylene-C₃₋₁₀ cycloaklyl, heterocyclyl, —C₁₋₆ alkylene-C₃₋₁₀ heterocyclyl, phenyl, heteroaryl, or NR^{h}R^{k}, where the alkyl, alkylene, cycloalkyl, heterocyclyl, phenyl, and heteroaryl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or 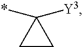
where Y³ is cyclopropyl, —CF₃, —OCF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, pipe rid in-1-yl, 4-hydroxy-pipe rid in-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, heteroarylene optionally substituted one or more times with substituents independently selected from R^{x}, or heterocyclylene optionally substituted one or more times with substituents independently selected from R^{x}; or the group -L-G is -cyano;
R¹ is hydrogen, R^{a}, phenyl, or heteroaryl, where the phenyl and heteroaryl groups are optionally substituted one or more times with substituents independently selected from R^{x};
R² is R^{b};
R³ is hydrogen, —C₁₋₆ alkyl, or —C₁₋₆ alkylene-C₃₋₁₀ cycloaklyl, where the alkyl, alkylene, and cycloalkyl groups are optionally substituted one or more times with substituents independently selected from R^{x};
R⁴ is —C₁₋₆ alkyl or —C₁₋₆ alkylene-C₃₋₁₀ cycloaklyl, where the alkyl, alkylene, and cycloalkyl groups are optionally substituted one or more times with substituents independently selected from R^{y};
R⁶ is hydrogen, —C₁₋₆ alkyl, —C₁₋₆ alkylene-C₃₋₁₀ cycloaklyl, where the alkyl, alkylene, and cycloalkyl groups are optionally substituted one or more times with substituents independently selected from R^{x};
R^{a} is
a) -halogen,
b) —C₁₋₄ alkyl,
c) —C₃₋₁₀ cycloalkyl,
d) -heterocyclyl,
e) -cyano,
f) —CF₃,
g) —OCF₃,
h) —O—R^{d},
i) —S(O)_{w}—R^{d},
j) —S(O)₂O—R^{d},
k) —NR^{d}R^{e},
l) —C(O)—R^{d},
m) —C(O)—O—R^{d},
n) —OC(O)—R^{d},
o) —C(O)NR^{d}R^{e},
p) —C(O)-heterocyclyl,
q) —NR^{d}C(O)R^{e},
r) —OC(O)NR^{d}R^{e},
s) —NR^{d}C(O)OR^{d}, or
t) —NR^{d}C(O)NR^{d}R^{e},
where the alkyl, cycloalkyl, and heterocyclyl groups are optionally substituted one or more times with substituents independently selected from R^{y};
R^{b} is
a) -halogen,
b) —C₁₋₄ alkyl,
c) —C₃₋₁₀ cycloalkyl,
d) -heterocyclyl,
e) -phenyl,
f) -heteroaryl,
g) -cyan,
h) —CF₃,
i) —OCF₃,
j) —O—R^{f},
k) —S(O)_{w}—R^{f},
l) —S(O)₂O—R^{f},
m) —NR^{f}R^{g},
n) —C(O)—R^{f},
o) —C(O)—O—R^{f},
p) —OC(O)—R^{f},
q) —C(O)NR^{f}R^{g},
r) —C(O)-heterocyclyl,
s) —NR^{f}C(O)R^{g},
t) —OC(O)NR^{f}R^{g},
u) —NR^{f}C(O)OR^{f}, or
v) —NR^{f}C(O)NR^{f}R^{g},
where the alkyl, cycloalkyl, heterocyclyl, phenyl, and heteroaryl groups are optionally substituted one or more times with substituents independently selected from R^{z};
R^{c} is
a) -halogen,
b) —C₁₋₄ alkyl,
c) —C₃₋₁₀ cycloalkyl,
d) -heterocyclyl,
e) -cyano,
f) —CF₃,
g) —OCF₃,
h) —O—R^{h},
i) —S(O)_{w}—R^{h},
j) —S(O)₂O—R^{h},
k) —NR^{h}R^{k},
l) —C(O)—R^{h},
m) —C(O)—O—R^{h},
n) —OC(O)—R^{h},
o) —C(O)NR^{h}R^{k},
p) —C(O)-heterocyclyl,
q) —NR^{h}C(O)R^{k},
r) —OC(O)NR^{h}R^{k},
s) —NR^{h}C(O)OR^{k},
t) —NR^{h}C(O)NR^{h}R^{k},
u) —NR^{h}S(O)_{w}R^{k},
v) -phenyl,
w) -heteroaryl, or
x) —O—(C₁₋₄ alkylene)-O—(C₁₋₄ alkylene)-N(R^{h})C(O)—OR^{k},
where the alkylene, alkyl, cycloalkyl, heterocyclyl, phenyl, and heteroaryl groups are optionally substituted one or more times with substituents independently selected from R^{x};
R^{d} and R^{e} are independently hydrogen, C₁₋₆ alkyl, or C₃₋₁₀ cycloalkyl, where the alkyl and cycloalkyl groups are optionally substituted one or more times with substituents independently selected from R^{y}; or, if R^{d} and R^{e} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{y};
R^{f} and R^{g} are independently hydrogen, C₁₋₆ alkyl, C₃₋₁₀ cycloalkyl, phenyl, or heteroaryl, where the alkyl, cycloalkyl, phenyl, and heteroaryl groups are optionally substituted one or more times with substituents independently selected from R^{z}; or, if R^{f} and R^{g} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{z};
R^{h} and R^{k} are independently hydrogen, C₁₋₆ alkyl, C₃₋₁₀ cycloalkyl, heterocyclyl, phenyl, or heteroaryl, where the alkyl, cycloalkyl, heterocyclyl, phenyl, and heteroaryl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x};
R^{y} is
a) -halogen,
b) —NH₂,
c) -cyano,
d) -carboxy,
e) -hydroxy,
f) -thiol,
g) —CF₃,
h) —OCF₃,
i) —C(O)—NH₂,
j) —S(O)₂—NH₂,
k) oxo,
l) —C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
m) -heterocyclyl optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
n) —C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
o) —O—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
p) —O—C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
q) —NH—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
r) —N(C₁₋₆ alkyl)₂ optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
s) —C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
t) —C(O)—O—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
u) —S—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
v) —S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
w) —C(O)—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
x) —C(O)—N(C₁₋₆ alkyl)₂, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
y) —S(O)₂—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
z) —S(O)₂—N(C₁₋₆ alkyl)₂, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
aa) —NH—C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂, or
bb) —NH—S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂;
R^{x} is
a) —R^{y}
b) -phenyl, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
c) -heteroaryl, optionally substituted one or more times with substituents selected independently from the group consisting of halogen, —OH, —O—C₁₋₆ alkyl, —NH₂, —NH—C₁₋₆ alkyl, and —N(C₁₋₆alkyl)₂,
d) —O-phenyl,
e) —O-heteroaryl,
f) —C(O)-phenyl,
g) —C(O)-heteroaryl,
h) —C(O)—O-phenyl, or
i) —C(O)—O-heteroaryl;
R^{z} is
a) —R^{y}
b) -phenyl,
c) -heteroaryl;
d) —O-phenyl,
e) —O-heteroaryl,
f) —C(O)-phenyl,
g) —C(O)-heteroaryl,
h) —C(O)—O-phenyl, or
i) —C(O)—O-heteroaryl;
v is an integer from 0 to 4, and
w is an integer from 0 to 2.

2. The method of claim 1, wherein the refractive ocular disorder is selected from the group consisting of presbyopia, myopia, hyperopia, astigmatism or a combination thereof.

3. The method of claim 2, wherein the refractive ocular disorder is presbyopia.

4. The method of claim 1, wherein near vision acuity of the subject is improved to 20/40 near vision or better.

5. The method of claim 1, wherein the subject has a gain of at least 10 letters in distance-corrected near visual acuity in the nondominant eye and/or as bilateral vision relative to vision prior to treatment.

6. The method of claim 1, wherein the subject has a gain of at least 2 correctly identified letters in distance-corrected near visual acuity in the nondominant eye or in bilateral vision relative to baseline.

7. The method of claim 1, wherein the subject has a gain of at least 60 ETDRS letters in binocular distance-corrected near visual acuity.

8. The method of claim 1, wherein the subject has at least a 5% improvement in binocular DCNVA relative to baseline.

9. The method of claim 1, wherein the subject has an improvement in Log MAR score of at least 0.050 points relative to baseline.

10. The method of claim 1, wherein prior to treatment the subject has Distance Corrected Near Visual Acuity of 20/40 or worse.

11. The method of claim 1, wherein prior to treatment the subject has best Corrected Distance Visual Acuity of 20/20 or better in at least one eye.

12. The method of claim 1, wherein the subject is a human.

13. The method of claim 1, wherein the compound of Formula (I) or a pharmaceutically acceptable salt thereof is administered orally or ocularly.

14. The method of claim 13, wherein the compound of Formula (I) or a pharmaceutically acceptable salt thereof is administered topically to the subject's eye.

15. The method of claim 1, wherein the compound of Formula (I) or a pharmaceutically acceptable salt thereof is administered between once and three times a day.

16. The method of claim 1, wherein the compound of Formula (I) is
1-Methyl-2-(6-trifluoromethoxy-benzothiazol-2-ylamino)-1H-benzoimidazole-6-carboxylic acid [2-(2-hydroxy-ethoxy)-ethyl]-amide.

17. The method of claim 1, wherein the compound of Formula (I) is
1-Methyl-2-(6-trifluoromethyl-benzothiazol-2-ylamino)-1H-benzimidazole-6-carboxylic acid dimethylcarbamoylmethyl-amide.

18. The method of claim 1, wherein the amount of the compound of Formula (I) or a pharmaceutically acceptable salt thereof administered is between 0.01 mg and 1000 mg.

19. The method of claim 1, wherein the compound of Formula (I) or a pharmaceutically acceptable salt thereof is administered ocularly in a formulation comprising between about 0.01% to about 20% w/v of compound of Formula (I) or a pharmaceutically acceptable salt thereof.

20. The method of claim 1, wherein a compound of Formula (I) or a pharmaceutically acceptable salt thereof is administered in combination with another active compound.

21. The method of claim 20, wherein the another active compound is lipoic acid or lipoic acid choline ester.

22. An ocular pharmaceutical composition comprising a compound of Formula (I) of claim 1, or a pharmaceutically acceptable salt thereof, and a pharmaceutically acceptable carrier.

23. The ocular pharmaceutical composition of claim 22 comprising
between about 0.01% to about 20% w/v of a compound of Formula (I) or a pharmaceutically acceptable salt thereof; and
a surfactant,
wherein the pH of the ocular pharmaceutical composition is between about 4 and 8.
